(19) United States
(12) Patent Application Publication
(10) Pub. No.: US 2026/0209358 A1
(43) Pub. Date: Jul. 23, 2026

(54) MULTIVALENT HEMATOPOIETIC CELL ENGAGERS OR ACTIVATORS

(71) Applicant: Radiant Biotherapeutics Inc., Montreal (CA)

(72) Inventors: Joanne Hulme, Toronto (CA); Peter Bayliss, Toronto (CA); Xinwen He, Toronto (CA); Yu-Fan Tsao, Toronto (CA)

(21) Appl. No.: 19/139,815

(22) PCT Filed: Dec. 18, 2023
(86) PCT No.: PCT/CA2023/051688

§ 371 Date: Jun. 16, 2025

Related U.S. Application Data
(60) Provisional application No. 63/433,467, filed on Dec. 18, 2022.

(51) Int. Cl. C07K 16/28; A61K 38/00; A61P 35/00; C07K 14/47

(52) U.S. Cl. CPC C07K 16/283; A61P 35/00; C07K 14/47; C07K 16/2803; C07K 16/2809; C07K 16/2851; C07K 16/2887; C07K 16/2896; A61K 38/00; C07K 2317/55; C07K 2317/92; C07K 2319/33

(57) ABSTRACT

Fusion polypeptides comprising: (1) a hematopoietic cell-binding moiety and (2) a nanocage monomer or subunit thereof; self-assembled polypeptide complexes comprising such fusion polypeptides. Self-assembled polypeptide complexes comprising Fc fusion polypeptides and fusion polypeptides disclosed herein and/or tumor-binding fusion polypeptides, and related compositions and methods.

### CROSS-REFERENCE TO RELATED APPLICATIONS

The present application claims the benefit of and priority to U.S. Provisional Patent Application No. 63/433,467 (filed Dec. 18, 2022), the entire contents of which are hereby incorporated by reference in its entirety for all purposes.

### SEQUENCE LISTING

The instant application contains a Sequence Listing which has been submitted electronically in XML format and is hereby incorporated by reference in its entirety. Said XML file, created on Dec. 15, 2023, is named “RBT051WO_SL.xml” and is 378 kilobytes in size.

### BACKGROUND

Immuno-oncology is a therapeutic approach that uses a patient's own immune system to fight cancer. Bispecific T cell engagers (BiTEs), bispecific antibodies which recognize both a T-cell and a tumor cell, are being developed as a therapeutic to bring T-cells in proximity with cancer cells, thereby facilitating T-cell-mediated tumor cell killing. However, development of BiTEs and similar therapeutics face challenges, including safety-related issues such as those associated with cytokine release syndrome.

The innate immune system also plays a key role in tumor immunosurveillance and generation of α-tumor immune response. Thus, similar approaches using bispecific antibodies to engage cells of the innate immune system, such as natural killer (NK) cells or myeloid cells, are also being developed to promote tumor cell killing. However, these approaches also face challenges, such as efficacy being tied to the level of expression of a target on the innate immune cell and the highly immunosuppressive microenvironment of tumors.

Thus, there is a need for improved therapeutics in the immuno-oncology space.

### SUMMARY

The present invention addresses this need with the provision of self-assembled polypeptide complexes which display IgG Fc fragments and one or both of (1) tumor cell-binders and (2) hematopoietic cell (such as NK cell, T cell, or myeloid cell) binders. Such self-assembled polypeptide complexes may be used to facilitate hematopoietic cell-mediated tumor killing. In the provided self-assembled polypeptide complexes, the relative ratios, valency, and orientation of the binders and fragments are tunable, allowing modulation of the function and characteristics of the complexes. Also disclosed are related fusion proteins, complexes, compositions, and methods.

In one aspect, provided are fusion polypeptides comprising: (1) a hematopoietic cell-binding moiety and (2) a nanocage monomer or subunit thereof.

In some embodiments, the hematopoietic cell-binding moiety comprises an antibody or antigen-binding fragment thereof.

In some embodiments, the antibody or antigen-binding fragment thereof comprises an immunoglobulin heavy chain variable region (V_{H}) and an immunoglobulin light chain variable region (V_{L} or V_{K}).

In some embodiments, the antibody or antigen-binding fragment thereof comprises a Fab fragment.

In some embodiments, the Fab fragment is a single-chain Fab fragment (scFab).

In some embodiments, the fusion polypeptide comprises a lymphoid cell-binding moiety.

In some embodiments, the lymphoid cell-binding moiety is a natural killer cell-binding moiety or T cell-binding moiety.

In some embodiments, the lymphoid cell-binding moiety is a natural killer cell-binding moiety.

In some embodiments, the natural killer cell-binding moiety is a CD16a-binding moiety, a NKp46-binding moiety, or a NKG2D-binding moiety.

In some embodiments, the natural killer cell-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a CD16a, NKp46, or NKG2D antibody.

In some embodiments, the CDRs of the V_{H} and the V_{L} or V_{K} have sequences which are identical to those of the heavy and light chain CDRs of a CD16a, NKp46, or NKG2D antibody, except for one or two amino acid substitutions total across all six CDRs.

In some embodiments, the CDRs of the V_{H} and the V_{L} or V_{K} are identical to those of the heavy and light chain CDRs of a CD16a, NKp46, or NKG2D antibody.

In some embodiments, the natural killer cell-binding moiety comprises an antibody fragment comprising heavy and light chain variable regions with sequences that have at least 85% identity to those of the heavy and light chain variable regions of a CD16a, NKp46, or NKG2D antibody.

In some embodiments, the lymphoid cell-binding moiety is a T cell-binding moiety.

In some embodiments, the T cell-binding moiety is a CD3-binding moiety.

In some embodiments, the T cell-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a CD3 antibody.

In some embodiments, the CDRs of the V_{H} and the V_{L} or V_{K} have sequences which are identical to those of the heavy and light chain CDRs of a CD3 antibody, except for one or two amino acid substitutions total across all six CDRs.

In some embodiments, the CDRs of the V_{H} and the V_{L} or V_{K} are identical to those of the heavy and light chain CDRs of a CD3 antibody.

In some embodiments, the T cell-binding moiety comprises an antibody fragment comprising heavy and light chain variable regions with sequences that have at least 85% identity to those of the heavy and light chain variable regions of a CD3 antibody.

In some embodiments, the fusion polypeptide comprises a myeloid cell-binding moiety.

In some embodiments, the myeloid cell-binding moiety is a SIRPα-binding moiety.

In some embodiments, the myeloid cell-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a SIRPα antibody.

In some embodiments, the CDRs of the V_{H} and the V_{L} or V_{K} have sequences which are identical to those of the heavy and light chain CDRs of a SIRPα antibody, except for one or two amino acid substitutions total across all six CDRs.

In some embodiments, the CDRs of the V_{H} and the V_{L} or V_{K} are identical to those of the heavy and light chain CDRs of a SIRPα antibody.

In some embodiments, the natural killer cell-binding moiety comprises an antibody fragment comprising heavy and light chain variable regions with sequences that have at least 85% identity to those of the heavy and light chain variable regions of a SIRPα antibody.

In some embodiments, the nanocage monomer is a ferritin monomer or a subunit thereof.

In some embodiments, the ferritin monomer is a human ferritin monomer.

In some embodiments, the ferritin monomer is a ferritin light chain.

In some embodiments, the hematopoietic cell-binding moiety is linked via an amino acid linker to the nanocage monomer or subunit thereof.

In some embodiments, the hematopoietic cell-binding moiety is linked via the N-terminus of the nanocage monomer or subunit thereof.

In one aspect, provided are self-assembled polypeptide complexes comprising: (a) a plurality of hematopoietic cell-binding fusion polypeptides, each hematopoietic cell-binding fusion polypeptide being a fusion polypeptide of as disclosed herein; and (b) a plurality of Fc fusion polypeptides, each Fc fusion polypeptide comprising (1) an Fc polypeptide and (2) a nanocage monomer or subunit thereof.

In some embodiments, the self-assembled polypeptide complex further comprises (c) a plurality of tumor-binding fusion polypeptides, each tumor-binding fusion polypeptide comprising (1) tumor-binding moiety and (2) a nanocage monomer or subunit thereof.

In some embodiments, within each Fc fusion polypeptide, the Fc polypeptide is linked via an amino acid linker to the nanocage monomer or subunit thereof.

In some embodiments, the Fc polypeptide is linked via the N-terminus of the nanocage monomer or subunit thereof.

In some embodiments, the Fc polypeptide is linked via the C-terminus of the nanocage monomer or subunit thereof.

In some embodiments, within each tumor-binding fusion polypeptide, the tumor-binding moiety is linked via an amino acid linker to the nanocage monomer or subunit thereof.

In some embodiments, the tumor-binding moiety is linked via the N-terminus of the nanocage monomer or subunit thereof.

In one aspect, provided are self-assembled polypeptide complexes comprising: (a) a plurality of hematopoietic cell-binding fusion polypeptides, each hematopoietic cell-binding fusion polypeptide being a fusion polypeptide as disclosed herein; and (b) a plurality of Fc fusion polypeptides, each Fc fusion polypeptide comprising (1) an Fc polypeptide linked via an amino acid linker to the C-terminus of (2) a nanocage monomer or subunit thereof.

In one aspect, provided are self-assembled polypeptide complexes comprising: (a) a plurality of tumor-binding fusion polypeptides, each tumor-binding fusion polypeptide comprising (1) tumor-binding moiety linked via an amino acid linker to (2) a nanocage monomer or subunit thereof; and (b) a plurality of Fc fusion polypeptides, each Fc fusion polypeptide comprising (1) an Fc polypeptide linked via an amino acid linker to the C-terminus of (2) a nanocage monomer or subunit thereof.

In one aspect, provided are self-assembled polypeptide complexes comprising: (a) a plurality of hematopoietic cell-binding fusion polypeptides, each hematopoietic cell-binding fusion polypeptide being a fusion polypeptide as disclosed herein; and (b) a plurality of tumor-binding fusion polypeptides, each tumor-binding fusion polypeptide comprising (1) tumor-binding moiety linked via an amino acid linker to (2) a nanocage monomer or subunit thereof; and (c) a plurality of Fc fusion polypeptides, each Fc fusion polypeptide comprising (1) an Fc polypeptide linked to the C-terminus of (2) a nanocage monomer or subunit thereof.

In some embodiments, the Fc polypeptide is linked to the C-terminus of a C-half nanocage monomer.

In some embodiments, the Fc polypeptide is linked to the C-terminus of a C-half ferritin.

In some embodiments, the tumor-binding moiety comprises an antibody or antigen-binding fragment thereof.

In some embodiments, the antibody or antigen-binding fragment thereof comprises an immunoglobulin heavy chain variable region (V_{H}) and an immunoglobulin light chain variable region (V_{L} or V_{K}).

In some embodiments, the antibody or antigen-binding fragment thereof comprises a Fab fragment.

In some embodiments, the Fab fragment is a single-chain Fab fragment (scFab).

In some embodiments, the tumor-binding moiety is a CD20-binding moiety or a CD37-binding moiety.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a CD20 or CD37 antibody.

In some embodiments, the CDRs of the V_{H} and the V_{L} or V_{K} have sequences which are identical to those of the heavy and light chain CDRs of a CD20 or CD37 antibody, except for one or two amino acid substitutions total across all six CDRs.

In some embodiments, the CDRs of the V_{H} and the V_{L} or V_{K} are identical to those of the heavy and light chain CDRs of a CD20 or CD37 antibody.

In some embodiments, the natural killer cell-binding moiety comprises an antibody fragment comprising heavy and light chain variable regions with sequences that have at least 85% identity to those of the heavy and light chain variable regions of a CD20 or CD37 antibody.

In some embodiments, the nanocage monomers within the Fc fusion polypeptide and within hematopoietic cell-binding fusion polypeptide and/or the tumor-binding fusion polypeptide are each a ferritin monomer or a subunit thereof.

In some embodiments, the ferritin monomer is a human ferritin monomer.

In some embodiments, the ferritin monomer is a ferritin light chain.

In some embodiments, the self-assembled polypeptide complex does not comprise any ferritin heavy chains or subunits of ferritin heavy chains.

In some embodiments, self-assembled polypeptide complex does not comprise any iron-binding moieties.

In one aspect, provided are a pharmaceutical compositions comprising a self-assembled polypeptide complex as disclosed herein and a pharmaceutically acceptable excipient.

In one aspect, provided are uses of a self-assembled polypeptide complex or a pharmaceutical composition as disclosed herein to treat, ameliorate, or prevent a disease or condition in a subject.

In one aspect, provided are a methods of treating, ameliorating, or preventing a disease or condition, the method comprising administering to a subject a self-assembled polypeptide complex or a pharmaceutical composition as disclosed herein.

In some embodiments, the subject is a mammal.

In some embodiments, the subject is human.

In some embodiments, the disease or condition is cancer, e.g., a B-cell lymphoma.

In one aspect, provided are fusion polypeptides comprising a contiguous full-length ferritin light chain having an N-terminus and a C-terminus, and (a) an antibody fragment fused via a first amino acid linker to the N-terminus and an Fc polypeptide fused via a second amino acid linker to the C-terminus; or (b) an antibody fragment fused via a first amino acid linker to the C-terminus and an Fc polypeptide fused via a second amino acid linker to the N-terminus.

In some embodiments, the antibody fragment is fused via a first amino acid linker to the N-terminus and an Fc polypeptide fused via a second amino acid linker to the C-terminus.

In some embodiments, the antibody fragment comprises an immunoglobulin heavy chain variable region (V_{H}) and an immunoglobulin light chain variable region (V_{L} or V_{K}).

In some embodiments, the antibody or antigen-binding fragment thereof comprises a Fab fragment.

In some embodiments, the Fab fragment is a single-chain Fab fragment (scFab).

In some embodiments, the antibody fragment is capable of binding to a tumor-associated antigen.

Also provided are self-assembled polypeptide complex comprising any of the afore-mentioned fusion polypeptides, as well as a pharmaceutical compositions comprising such self-assembled polypeptide complexes and a pharmaceutically acceptable excipient, and methods of treating, ameliorating, or preventing a disease or condition, the method comprising administering to a subject such self-assembled polypeptide complexes or pharmaceutical excipients.

Also provided are methods comprising a step of contacting a tumor cell with a self-assembled polypeptide complex as disclosed herein. In some embodiments, the step of contacting results in apoptosis of the tumor cell, e.g., a tumor cell that expresses CD37 can be contacted with a self-assembled polypeptide complex which comprises a fusion protein comprising an α-CD37 antibody or fragment thereof (such as a Fab). In some embodiments, apoptosis is induced with an EC50 value of less than 1 nM for the self-assembled polypeptide complex. In some embodiments, the step of contacting results in antibody-dependent cellular cytotoxicity (ADCC) of the tumor cell, e.g., with an EC50 value of less than 5 pM for the self-assembled polypeptide complex.

In some embodiments, the step of contacting is conducted in the presence of complement proteins and results in complement-dependent cytotoxicity (CDC) of the tumor cell, e.g., with an EC50 value of less than 0.4 nM for the self-assembled polypeptide complex. In some embodiments, the step of contacting results in antibody-dependent cellular phagocytosis (ADCP) of the tumor cell, e.g., with an EC50 value of less than 18 pM for the self-assembled polypeptide complex. In some embodiments, the step of contacting occurs inside a mammal having a tumor, e.g., a human.

Also provided are self-assembled polypeptide complexes as described herein and which, when contacted with a tumor cell, are capable of inducing an effect selected from the group consisting of
(a) apoptosis of the tumor cell with an EC50 value of less than 1 nM;
(b) ADCC of the tumor cell with an EC50 value of less than 1 nM;
(c) CDC of the tumor cell, when the self-assembled polypeptide complex is contacted with the tumor cell in the presence of complement, with an EC50 value of less than 0.4 nM;
(d) ADCP with an EC50 value of less than 0.1 nM and
(e) any combination of the foregoing.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a diagrammatic representation of human ferritin light chain (hFTL) and example N-half ferritin (N-hFTL) and C-half ferritin (C-hFTL) molecules.

FIGS. 1B and 1C are diagrammatic representations of sets of fusion polypeptides that together may form exemplary Multabodies (MBs) of the disclosure.

FIG. 2A is a diagrammatic representation of fusion proteins used in MBs of Format 1.

FIG. 2B is a schematic illustrating the orientation of a single Fc (including its FcγR-binding and FcRn-binding pockets) relative to a light chain ferritin complex (lower part of FIG. 2B) within an MB of Format 1. The diagram is not to scale. For ease of illustration, no Fabs are depicted, and only a single Fc is depicted.

FIG. 3A is a diagrammatic representation of fusion proteins used in MBs of Format 2.

FIG. 3B is a schematic illustrating the orientation of a single Fc (including its FcγR-binding and FcRn-binding pockets) relative to a light chain ferritin complex (lower part of FIG. 2B) within a Multabody of Format 1. The diagram is not to scale. For ease of illustration, no Fabs are depicted, and only a single Fc is depicted.

FIG. 4 is schematic depiction of a reporter assay for antibody-dependent cellular cytotoxicity (ADCC).

FIG. 5 is plot showing fold of ADCC induction (y-axis) vs. log concentration of various MBs (x-axis): Format 1 αCD37/Fc MBs, Format 2 αCD37/Fc MBs, and Format 2 control Fc MBs.

FIG. 6 depicts plots showing the percentage of single cells present within the FITC-Annexin-V+ gate as determined by flow cytometry (y-axis) vs. the concentration of Format 2 αCD37/Fc MBs (squares) or parent IgG (circles) incubated with target Ramos cells for 24 hours (x-axis).

FIG. 7 depicts plots showing the percentage of specific lysis of cells (y-axis) vs. the log concentration of Format 2 αCD37/Fc MBs (squares) or control α-respiratory syncytial virus (RSV) MB (circles) incubated with target Ramos cells and primary human NK cells for 4 hours (x-axis).

FIG. 8 is an ELISA plot showing the absorbance as measured at 450 nm (y-axis) vs. the log concentration of Format 2 αCD37/Fc MBs (triangles) or parent IgG (circles) coated on a 96-well plate and incubated with recombinant human C1q for 1 hour (x-axis).

FIG. 9 depicts plots showing the percentage of single cells present within the propidium iodide (PI)+ gate as determined by flow cytometry (y-axis) vs. the log concentration of Format 2 αCD37/Fc MBs (squares) or parental IgGs (circles) incubated with target Daudi cells for 2 hours in the presence of complement proteins (x-axis).

FIG. 10 depicts plots showing the percentage of single cells present within the propidium iodide (PI)+ gate as determined by flow cytometry (y-axis) vs. the log concentration of Format 2 αCD37/Fc MBs (squares) or control α-respiratory syncytial virus (RSV) MBs (circles) incubated with target Daudi cells for 2 hours in the presence of complement proteins (x-axis).

FIG. 11 depicts plots showing the percentage of pHrodo Green AM/CellTrace Violet double positive cells in all live single cells as determined by flow cytometry (y-axis) vs. the log concentration of Format 2 αCD37/Fc MBs (squares) or control α-respiratory syncytial virus (RSV) MBs (circles) incubated with target Ramos cells and THP-1 cells for 1 hour (x-axis).

FIG. 12A is a diagrammatic representation of fusion proteins used in Format 2 αCD37/Fc MBs.

FIG. 12B is a diagrammatic representation of fusion proteins used in CD37/NKp46 bispecific MBs.

FIGS. 13A-13C depict plots showing the percentage of specific lysis of cells (y-axis) vs. the log concentration of CD37/NKp46 bispecific MBs (squares) or control CD37 monospecific MBs (circles) incubated with target Ramos cells and primary human NK cells for 4 hours (x-axis).

FIGS. 14A-14C depict plots showing the percentage of single cells present within the FITC-Annexin-V+ gate as determined by flow cytometry (y-axis) vs. the concentration of CD37/NKp46 bispecific MBs (squares) or control CD37 monospecific MBs (circles) incubated with target Daudi cells for 24 hours (x-axis).

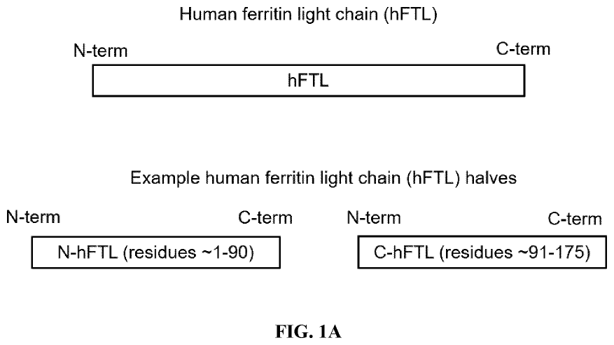

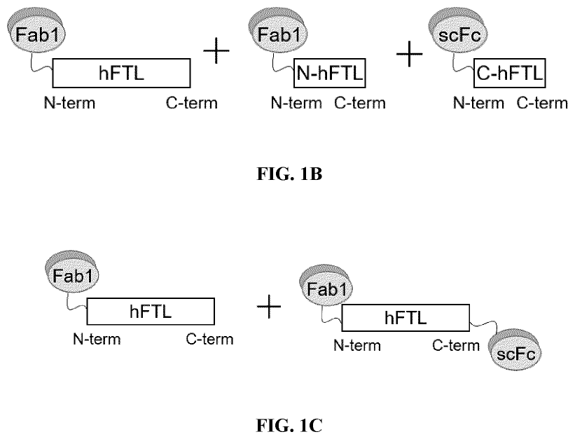

### DETAILED DESCRIPTION

Disclosed herein are fusion polypeptides each comprising (1) a nanocage monomer or subunit thereof and (2) an antibody fragment (such as a hematopoietic cell-binder or a tumor-cell binder) or an Fc polypeptide. The nanocage monomers or subunits thereof drive self-assembly of the fusion polypeptides into complexes which display one or more of the aforementioned cell-binders or tumor-binders and Fc polypeptides.

### Definitions

The terms “about” and “approximately,” when used herein in reference to a value, are used interchangeably and refer to a value that is similar to the referenced value. In general, those skilled in the art, familiar with the context, will appreciate the relevant degree of variation encompassed by “about” or “approximately” in that context. For example, in some embodiments, the terms “about” and “approximately” may encompass a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the terms “alter,” “altered,” “decrease,” “decreased,” “increase,” “increased,” or “reduction,” “reduced,” (e.g., in reference to certain outcomes or effects) have meanings relative to a reference level. In some embodiments, in the context of discussing mutations in an Fc chain or Fc polypeptide, the reference level is a level known or as determined with an IgG that does not contain the referenced mutation(s) in the Fc region.

As used herein, the term “antigen binding fragment” of an antibody, refers to one or more fragments of an antibody that retain the ability to specifically bind to an antigen. Examples of binding fragments encompassed within the term “antigen binding fragment” of an antibody include a Fab fragment, a F(ab′) 2 fragment, a Fd fragment, a Fv fragment, a scFv fragment, a dAb fragment (Ward et al., (1989) Nature 341:544-546), and an isolated complementarity determining region (CDR). In some embodiments, an “antigen binding fragment” comprises a heavy chain variable region and a light chain variable region. These antibody fragments can be obtained using conventional techniques known to those with skill in the art, and the fragments can be screened for utility in the same manner as are intact antibodies.

As used herein, the term “binding,” unless otherwise specified, refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell). As used herein, the phrases “non-binding” or “no binding,” or similar phrases, between two entities refers to 1) a lack of detectable binding or 2) binding below a set threshold that corresponds to no binding in an appropriate assay, e.g., an in vitro binding assay such as biolayer interferometry, surface plasmon resonance, a cell binding assay such as flow cytometry, or enzyme-linked immunosorbent assay (ELISA). For example, in some embodiments, in an in vitro biolayer interferometry assay, a maximal association binding response of less than 0.1 nm after 180 seconds to a biosensor loaded with 0.8 nm of target when the test article is present at a concentration of 20 nM is classified as “non-binding.”

The terms “ferritin” and “apoferritin” are used interchangeably herein and generally refer to a polypeptide (e.g., a ferritin chain) that is capable of assembling into a ferritin complex which typically comprises 24 protein subunits. In some embodiments, the ferritin is a human ferritin, e.g., a human ferritin light chain, e.g., a human ferritin light chain having at least 85% sequence identity to SEQ ID NO:1 or UniProt P02792. In some embodiments, the ferritin is a wild-type ferritin. For example, the ferritin may be a wild-type human ferritin.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains.

As used herein, the term “linker” is used to refer to an entity that connects two or more elements to form a multi-element agent. For example, those of ordinary skill in the art appreciate that a polypeptide (e.g., fusion polypeptide) whose structure includes two or more functional or organizational domains often includes a stretch of amino acids between such domains that links them to one another. In some embodiments, a polypeptide comprising a linker element has an overall structure of the general form S1-L-S2, wherein S1 and S2 may be the same or different and represent two domains associated with one another by the linker (L). In some embodiments, the linker is an “amino acid linker,” that is, it comprises amino acid residues, e.g., an amino acid linker may comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100 or more amino acid residues. In some embodiments, a linker is characterized in that it tends not to adopt a rigid three-dimensional structure, but rather provides flexibility to the polypeptide.

The term “multispecific,” as used herein, refers to the characteristic of having at least two binding sites at which at least two different binding partners, e.g., an antigen or receptor (e.g., Fc receptor), can bind. For example, a polypeptide complex that comprises at least two Fab fragments, wherein each of the two Fab fragments is capable of binding (e.g., specifically binding) to a different antigen, is “multispecific.” As an additional example, a polypeptide complex that comprises an Fc fragment (which is capable of binding (e.g., specifically binding) to an Fc receptor) and a Fab fragment (which is capable of binding (e.g., specifically binding) to an antigen) is “multispecific.”

The term “multivalent,” as used herein, refers to the characteristic of having at least two binding sites at which a binding partner, e.g., an antigen or receptor (e.g., Fc receptor), can bind. The binding partners that can bind to at least two binding sites may be the same or different.

The term “nanocage monomer,” as used herein, refers to a single chain of a polypeptide that is capable of self-assembling with other nanocage monomers to form a self-assembled polypeptide complex comprising a plurality of nanocage monomers. In some embodiments, the nanocage monomer is selected from monomers of ferritin, apoferritin, encapsulin, sulfur oxygenase reductase (SOR), lumazine synthase, pyruvate dehydrogenase, carboxysome, vault proteins, GroEL, heat shock protein, E2P coat protein, MS2 coat protein, fragments thereof, and variants thereof.

The term “polypeptide,” as used herein, generally has its art-recognized meaning of a polymer of at least three amino acids, e.g., linked to each other by peptide bonds. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, glycosylation etc. In some embodiments, proteins may comprise natural amino acids, non-natural amino acids, synthetic amino acids, and combinations thereof.

As used herein, the term “specifically binds, “specifically binding,” “binds specifically,” or similar terms, means that a binding moiety (e.g., an antibody or an antigen-binding fragment thereof) forms a complex with an antigen that is relatively stable under physiologic conditions. Specific binding can be characterized by an equilibrium dissociation constant of 1×10⁻⁶ M or less, 1×10⁻⁷ M or less, 1×10⁻⁸ M or less, or 1×10⁻⁹M or less (e.g., a smaller K_{D} denotes a tighter binding). Methods for determining whether two molecules specifically bind are well known in the art and include, for example, equilibrium dialysis, surface plasmon resonance, enzyme-linked immunosorbent assay, or biolayer interferometry measurements, etc. In some embodiments, “specifically binds” and similar terms refers to a characteristic of the binding moiety in that the binding moiety is capable of binding to a target antigen but does is not capable of binding to other antigens such as distantly related family members of the antigen.

The term “self-assembled,” when used in reference to a macromolecular complex (e.g., a polypeptide complex), refers to the spontaneous formation of that complex when sufficient constituents of the complex (e.g., fusion polypeptides) to be formed are present. In some embodiments, complexes self-assemble in physiological conditions, or in a buffer (e.g., a solution) that corresponds to physiological conditions.

As used herein, the term “subject” refers to an organism, typically a mammal (e.g., a human). In some embodiments, a subject is suffering from or susceptible to a relevant disease, disorder or condition. In some embodiments, a subject displays one or more symptoms or characteristics of a disease, disorder or condition. In some embodiments, a subject is someone with one or more features characteristic of susceptibility to or risk of a disease, disorder, or condition. In some embodiments, a subject is a patient. In some embodiments, a subject is a subject to whom diagnosis and/or therapy is and/or has been administered.

As used herein, the term “treatment” (also “treat” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively, or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### A. Fusion Polypeptides

In many embodiments, fusion polypeptides compatible with compositions and methods disclosed herein generally comprise (1) a nanocage monomer (or subunit thereof) as described herein and (2) a cell-binder (such as a hematopoietic cell-binder or a tumor-cell binder) or an Fc polypeptide, which may be linked via a linker, such as a linker described herein.

### 1. Nanocage Monomers and Subunits Thereof.

In some embodiments, the nanocage monomer is a ferritin monomer.

The term “ferritin monomer,” is used herein to refer to a single chain of a ferritin that, in the presence of other ferritin chains, is capable of self-assembling into a polypeptide complex comprising a plurality of ferritin chains, e.g., 24 or more ferritin chains. In some embodiments, the ferritin monomer is a ferritin light chain. In some embodiments, the ferritin monomer does not include a ferritin heavy chain or other ferritin components capable of binding to iron or capable of ferroxidase activity.

In some embodiments, each fusion polypeptide within the self-assembled polypeptide complex comprises a ferritin light chain or a subunit of a ferritin light chain. In these embodiments, the self-assembled polypeptide complex does not comprise any ferritin heavy chains or subunits of ferritin heavy chains. In such embodiments, the self-assembled polypeptide complex does not comprise and is not associated with Fe³⁺, an iron ion normally carried by ferritin complexes which comprise the ferritin heavy chain.

In some embodiments, the ferritin monomer is a human ferritin chain, e.g., a human ferritin light chain, e.g., a human ferritin light chain having the sequence of at least residues 2-175 of SEQ ID NO:1.

As used herein, a “full-length ferritin light chain” refers to a ferritin polypeptide that is capable of forming a four-helix bundle of two couples of α-parallel α-helices connected by a loop and further comprising a C-terminal α-helix. In some embodiments, a full-length ferritin light chain comprises at least 85%, at least 90%, at least 95%, or 100% of the length of an amino acid sequence of SEQ ID NO: 1. In some embodiments, a full-length ferritin light chain has an amino acid sequence which has at least 85%, at least 90%, or at least 95% sequence identity to the SEQ ID NO: 1. In some embodiments, a full-length ferritin light chain has an amino acid sequence of SEQ ID NO:1. In some embodiments, the full-length ferritin light chain is a single contiguous ferritin polypeptide.

A “subunit” of a ferritin monomer refers to a portion of a ferritin monomer that is capable of spontaneously associating with another, distinct subunit of a ferritin monomer, so that the subunits together form a ferritin monomer, which ferritin monomer, in turn, is capable of self-assembling with other ferritin monomers to form a polypeptide complex.

In some embodiments, the ferritin monomer subunit comprises approximately half of a ferritin monomer. As used herein, the term “N-half ferritin” refers to approximately half of a ferritin chain, which half comprises the N-terminus of the ferritin chain. As used herein, the term “C-half ferritin” refers to approximately half a ferritin chain, which half comprises the C-terminus of the ferritin chain. The exact point at which a ferritin chain may be divided to form the N-half ferritin and the C-half ferritin may vary depending on the embodiment. In the context of ferritin monomer subunits based on human ferritin light chain, for example, the halves may be divided at a point that corresponds to a position between about position 75 to about position 100 of SEQ ID NO:1 (or a substantial portion thereof). For example, in some embodiments, an N-half ferritin based on a human ferritin light chain has an amino acid sequence corresponding to residues 1-95 of SEQ ID NO: 1 (or a substantial portion thereof, e.g., residues 2-95 of SEQ ID NO: 1), and a C-half ferritin based on a human ferritin light chain has an amino acid sequence corresponding to residues 96-175 of SEQ ID NO: 1 (or a substantial portion thereof).

In some embodiments, the halves are divided at a point that corresponds to a position between about position 85 to about position 92 of SEQ ID NO: 1. For example, in some embodiments, an N-half ferritin based on a human ferritin light chain has an amino acid sequence corresponding to residues 1-90 of SEQ ID NO:1 (or a substantial portion thereof, e.g., residues 2-90 of SEQ ID NO: 1), and a C-half ferritin based on a human ferritin light chain has an amino acid sequence corresponding to residues 91-175 of SEQ ID NO:1 (or a substantial portion thereof.

### 2. Binding Moieties

Binding moieties (e.g., hematopoietic cell-binding moieties or tumor-binding moieties) typically comprise an antibody fragment.

In some embodiments, the antibody fragment is a Fab. In some embodiments, the antibody fragment is a single-chain Fab (scFab); for example, a fusion polypeptide comprising both the heavy and light chains of a Fab, optionally linked by a linker (e.g., amino acid linker as disclosed herein) is used.

In certain embodiments, the antibody fragment comprises a heavy chain variable region (e.g., a V_{H}). In certain embodiments, the antibody fragment comprises a heavy chain variable domain (e.g., V_{H}) and a light chain variable domain (e.g., a V_{L} or V_{K}). In certain embodiments, the antibody fragment comprises a Fab which comprises a heavy chain variable domain (e.g., V_{H}) and a light chain variable domain (e.g., a V_{L} or V_{K}).

In certain embodiments, the antibody fragment does not comprise any domains from the Fc region, e.g., does not comprise any constant heavy (CH2 or CH3) domains. In some embodiments, the antibody fragment is an antibody fragment of, or derived from, a fully human or humanized antibody. In some embodiments, the antibody fragment is an antibody fragment of, or derived from, a chimeric antibody. The antibody from which the antibody fragment is obtained or derived can be of any of a variety of antibody classes, including, e.g., an IgG1 antibody, an IgG2 antibody, or an IgG4 antibody. In some embodiments, the antibody fragment is obtained or derived from an agonistic antibody, e.g., an agonistic humanized antibody.

In embodiments where multiple types of fusion polypeptides having antibody fragments are used, the antibody fragments in the various types of fusion polypeptides may be capable of binding to the same epitope on a given antigen (e.g., an antigen on a hematopoietic cell or a tumor-associated antigen), capable of binding to epitopes that are distinct and non-overlapping on an antigen, or capable of binding to epitopes that are distinct but overlapping on the same antigen.

### A. Hematopoietic Cell-Binding Moieties

Hematopoietic cell-binding moieties are generally capable of binding (e.g., specifically binding) to an antigen on a hematopoietic cell (e.g., lymphoid cell (such as a natural killer (NK) cell or a T-cell) or a myeloid cell).

### Lymphoid Cell-Binding Moieties

In some embodiments, the hematopoietic cell-binding moiety is a lymphoid cell-binding moiety, e.g., a natural killer (NK) cell-binding moiety or T cell-binding moiety.

### NK Cell-Binding Moieties

In some embodiments, the natural killer cell-binding moiety is capable of binding (e.g., specifically binding) to CD16a, NKp46, or NKG2D.

In some embodiments, the natural killer cell-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which are similar to (e.g., each having at most one or two amino acids differing from) those of the heavy and light chain CDRs of a CD16a, NKp46, or NKG2D antibody (e.g., a human or humanized CD16a, NKp46, or NKG2D antibody).

In some embodiments, the natural killer cell-binding moiety is an antibody fragment comprising a heavy chain variable region (V_{H}) and light chain variable region (V_{L} or V_{K}), wherein the CDRs of the V_{H} and the V_{L} or V_{K} have sequences which are identical to those of the heavy and light chain CDRs of a CD16a, NKp46, or NKG2D antibody (e.g., a human or humanized CD16a, NKp46, or NKG2D antibody), except for one or two amino acid substitutions total across all six CDRs.

In some embodiments, the natural killer cell-binding moiety is an antibody fragment comprising a heavy chain variable region (V_{H}) and light chain variable region (V_{L} or V_{K}), wherein the CDRs of the V_{H} and the V_{L} or V_{K} are identical to those of the heavy and light chain CDRs of a CD16a, NKp46, or NKG2D antibody (e.g., a human or humanized CD16a, NKp46, or NKG2D antibody).

In some embodiments, the natural killer cell-binding moiety is an antibody fragment comprising heavy and light chain variable regions (e.g., V_{H} and V_{L} or V_{K}) with sequences that have at least 85%, at least 87.5%, at least 90%, at least 91%, at least 92%, at least 93%, at least 94%, at least 95%, at least 96%, at least 97%, at least 98% or at least 99% identity to those of the heavy and light chain variable regions of a CD16a, NKp46, or NKG2D antibody (e.g., a human or humanized CD16a, NKp46, or NKG2D antibody). In some embodiments, the natural killer cell-binding moiety is an antibody fragment comprising heavy and light chain variable regions (e.g., V_{H} and V_{L} or V_{K}) with sequences identical to those of the heavy and light chain variable regions of a CD16a, NKp46, or NKG2D antibody (e.g., a human or humanized CD16a, NKp46, or NKG2D antibody).

Non-limiting examples of CD16a antibodies include those whose heavy chain variable region, light chain variable region, and CDR sequences are depicted in Tables 1A (V_{H}1/V_{L}1) and 1B (V_{H}2/V_{L}1).

Non-limiting examples of NKp46 antibodies include those whose heavy chain variable region, light chain variable region, and CDR sequences are depicted in Tables 2A (Clone 1), 2B (Clone 3), 2C (NKp46-1 H1L1), 2D (NKp46-2 H1L1), 2E (NKp46-3 H1L1), 2F (NKp46-4H1L2), and 2G (09).

Non-limiting examples of NKG2D antibodies include tesnatilimab and others whose heavy chain variable region, light chain variable region, and CDR sequences are depicted in Tables 3A (A49), 3B (E79), and 3C (tesnatilimab).

TABLE 1A
CD16A Antibody Sequences (VH1/VL1)

| Heavy chain variable region | | Light chain variable region | |
|---|---|---|---|
| QVQLVQSGAEVKKPGESLKVSCKASGYTFTSY | | SYVLTQPSSVSVAPGQTATISCGGHNIGSK | |
| YMHWVRQAPGQGLEWMGAIEPMYGSTSYAQKF | | NVHWYQQRPGQSPVLVIYQDNKRPSGIPER | |
| QGRVTMTRDTSTSTVYMELSSLRSEDTAVYYC | | FSGSNSGNTATLTISGTQAMDEADYYCQVW | |
| ARGSAYYYDFADYWGQGTLVTVSS | | DNYSVLFGGGTKLTVL | |
| (SEQ ID NO: 8) | | (SEQ ID NO: 9) | |
| CDR Sequences (Kabat) | | CDR Sequences (Kabat) | |
| CDR-H1 | SYYMH | CDR-L1 | GGHNIGSKNVH |
| | (SEQ ID NO: 10) | | (SEQ ID NO: 13) |
| CDR-H2 | AIEPMYGSTSYAQKFQG | CDR-L2 | QDNKRPS |
| | (SEQ ID NO: 11) | | (SEQ ID NO: 14) |
| CDR-H3 | GSAYYYDFADY | CDR-L3 | QVWDNYSVL |
| | (SEQ ID NO: 12) | | (SEQ ID NO: 15) |
| CDR Sequences (IMGT) | | CDR Sequences (IMGT) | |
| CDR-H1 | GYTFTSYY | CDR-L1 | NIGSKN |
| | (SEQ ID NO: 16) | | (SEQ ID NO: 19) |
| CDR-H2 | IEPMYGST | CDR-L2 | QD |
| | (SEQ ID NO: 17) | | |
| CDR-H3 | ARGSAYYYDFADY | CDR-L3 | QVWDNYSVL |
| | (SEQ ID NO: 18) | | (SEQ ID NO: 20) |
| CDR Sequences (Chothia) | | CDR Sequences (Chothia) | |
| CDR-H1 | GYTFTSY | CDR-L1 | GGHNIGSKNVH |
| | (SEQ ID NO: 21) | | (SEQ ID NO: 24) |
| CDR-H2 | EPMYGS | CDR-L2 | QDNKRPS |
| | (SEQ ID NO: 22) | | (SEQ ID NO: 25) |
| CDR-H3 | GSAYYYDFADY | CDR-L3 | QVWDNYSVL |
| | (SEQ ID NO: 23) | | (SEQ ID NO: 26) |

TABLE 1B
CD16A Antibody Sequences (VH2/VL1)

| Heavy chain variable region | | Light chain variable region | |
|---|---|---|---|
| QVQLVQSGAEVKKPGESLKVSCKASGYTFTSY | | SYVLTQPSSVSVAPGQTATISCGGHNIGSK | |
| YMHWVRQAPGQGLEWMGIINPSGGSTSYAQKF | | NVHWYQQRPGQSPVLVIYQDNKRPSGIPER | |
| QGRVTMTRDTSTSTVYMELSSLRSEDTAVYYC | | FSGSNSGNTATLTISGTQAMDEADYYCQVW | |
| ARGSAYYYDFADYWGQGTLVTVSS | | DNYSVLFGGGTKLTVL | |
| (SEQ ID NO: 27) | | (SEQ ID NO: 28) | |
| CDR Sequences (Kabat) | | CDR Sequences (Kabat) | |
| CDR-H1 | SYYMH | CDR-L1 | GGHNIGSKNVH |
| | (SEQ ID NO: 29) | | (SEQ ID NO: 32) |
| CDR-H2 | IINPSGGSTSYAQKFQG | CDR-L2 | QDNKRPS |
| | (SEQ ID NO: 30) | | (SEQ ID NO: 33) |
| CDR-H3 | GSAYYYDFADY | CDR-L3 | QVWDNYSVL |
| | (SEQ ID NO: 31) | | (SEQ ID NO: 34) |
| CDR Sequences (IMGT) | | CDR Sequences (IMGT) | |
| CDR-H1 | GYTFTSYY | CDR-L1 | NIGSKN |
| | (SEQ ID NO: 35) | | (SEQ ID NO: 38) |
| CDR-H2 | INPSGGST | CDR-L2 | QD |
| | (SEQ ID NO: 36) | | |
| CDR-H3 | ARGSAYYYDFADY | CDR-L3 | QVWDNYSVL |
| | (SEQ ID NO: 37) | | (SEQ ID NO: 39) |
| CDR Sequences (Chothia) | | CDR Sequences (Chothia) | |
| CDR-H1 | GYTFTSY | CDR-L1 | GGHNIGSKNVH |
| | (SEQ ID NO: 40) | | (SEQ ID NO: 43) |
| CDR-H2 | NPSGGS | CDR-L2 | QDNKRPS |
| | (SEQ ID NO: 41) | | (SEQ ID NO: 44) |
| CDR-H3 | GSAYYYDFADY | CDR-L3 | QVWDNYSVL |
| | (SEQ ID NO: 42) | | (SEQ ID NO: 45) |

TABLE 2A
NKp46 Antibody Sequences (Clone 1)

| Heavy chain variable region | | Light chain variable region | |
|---|---|---|---|
| QVQLVQSGAEVKKPGSSVKVSCKASGYTFSDY | | DIQMTQSPSSLSASVGDRVTITCRASQDIS | |
| VINWVRQAPGQGLEWMGEIYPGSGTNYYNEKF | | NYLNWYQQKPGKAPKLLIYYTSRLHSGVPS | |
| KAKATITADKSTSTAYMELSSLRSEDTAVYYC | | RFSGSGSGTDFTFTISSLQPEDIATYFCQQ | |
| ARRGRYGLYAMDYWGQGTTVTVSS | | GNTRPWTFGGGTKVEIK | |
| (SEQ ID NO: 46) | | (SEQ ID NO: 47) | |
| CDR Sequences (Kabat) | | CDR Sequences (Kabat) | |
| CDR-H1 | DYVIN | CDR-L1 | RASQDISNYLN |
| | (SEQ ID NO: 48) | | (SEQ ID NO: 51) |
| CDR-H2 | EIYPGSGTNYYNEKFKA | CDR-L2 | YTSRLHS |
| | (SEQ ID NO: 49) | | (SEQ ID NO: 52) |
| CDR-H3 | RGRYGLYAMDY | CDR-L3 | QQGNTRPWT |
| | (SEQ ID NO: 50) | | (SEQ ID NO: 53) |
| CDR Sequences (IMGT) | | CDR Sequences (IMGT) | |
| CDR-H1 | GYTFSDYV | CDR-L1 | QDISNY |
| | (SEQ ID NO: 54) | | (SEQ ID NO: 57) |
| CDR-H2 | IYPGSGTN | CDR-L2 | YT |
| | (SEQ ID NO: 55) | | |
| CDR-H3 | ARRGRYGLYAMDY | CDR-L3 | QQGNTRPWT |
| | (SEQ ID NO: 56) | | (SEQ ID NO: 58) |
| CDR Sequences (Chothia) | | CDR Sequences (Chothia) | |
| CDR-H1 | GYTFSDY | CDR-L1 | RASQDISNYLN |
| | (SEQ ID NO: 59) | | (SEQ ID NO: 62) |
| CDR-H2 | YPGSGT | CDR-L2 | YTSRLHS |
| | (SEQ ID NO: 60) | | (SEQ ID NO: 63) |
| CDR-H3 | RGRYGLYAMDY | CDR-L3 | QQGNTRPWT |
| | (SEQ ID NO: 61) | | (SEQ ID NO: 64) |

TABLE 2B
NKp46 Antibody Sequences (Clone 3)

| Heavy chain variable region | | Light chain variable region | |
|---|---|---|---|
| QVQLVQSGAEVKKPGSSVKVSCKASGYTFSEY | | EIVMTQSPATLSVSPGERATLSCRASQSIS | |
| TMHWVRQAPGQGLEWMGGISPNIGGTSYNQKF | | DYLHWYQQKPGQAPRLLIKYASQSISGIPA | |
| KGRVTITADKSTSTAYMELSSLRSEDTAVYYC | | RFSGSGSGTDFTLTISSLEPEDFAVYYCQN | |
| ARRGGSFDYWGQGTTVTVSS | | GHSFPLTFGQGTKLEIK | |
| (SEQ ID NO: 65) | | (SEQ ID NO: 66) | |
| CDR Sequences (Kabat) | | CDR Sequences (Kabat) | |
| CDR-H1 | EYTMH | CDR-L1 | RASQSISDYLH |
| | (SEQ ID NO: 67) | | (SEQ ID NO: 70) |
| CDR-H2 | GISPNIGGTSYNQKFKG | CDR-L2 | YASQSIS |
| | (SEQ ID NO: 68) | | (SEQ ID NO: 71) |
| CDR-H3 | RGGSFDY | CDR-L3 | QNGHSFPLT |
| | (SEQ ID NO: 69) | | (SEQ ID NO: 72) |
| CDR Sequences (IMGT) | | CDR Sequences (IMGT) | |
| CDR-H1 | GYTFSEYT | CDR-L1 | QSISDY |
| | (SEQ ID NO: 73) | | (SEQ ID NO: 76) |
| CDR-H2 | ISPNIGGT | CDR-L2 | YA |
| | (SEQ ID NO: 74) | | |
| CDR-H3 | ARRGGSFDY | CDR-L3 | QNGHSFPLT |
| | (SEQ ID NO: 75) | | (SEQ ID NO: 77) |
| CDR Sequences (Chothia) | | CDR Sequences (Chothia) | |
| CDR-H1 | GYTFSEY | CDR-L1 | RASQSISDYLH |
| | (SEQ ID NO: 78) | | (SEQ ID NO: 81) |
| CDR-H2 | SPNIGG | CDR-L2 | YASQSIS |
| | (SEQ ID NO: 79) | | (SEQ ID NO: 82) |
| CDR-H3 | RGGSFDY | CDR-L3 | QNGHSFPLT |
| | (SEQ ID NO: 80) | | (SEQ ID NO: 83) |

TABLE 2C
NKp46 Antibody Sequences (NKp46-1 H1L1)

| Heavy chain variable region | | Light chain variable region | |
|---|---|---|---|
| QVQLVQSGAEVKKPGSSVKVSCKASGYTFSDY | | DIQMTQSPSSLSASVGDRVTITCRASQDIS | |
| VINWVRQAPGQGLEWMGEIYPGSGTNYYNEKF | | NYLNWYQQKPGKAPKLLIYYTSRLHSGVPS | |
| KAKATITADKSTSTAYMELSSLRSEDTAVYYC | | RFSGSGSGTDFTFTISSLQPEDIATYFCQQ | |
| ARRGRYGLYAMDYWGQGTTVTVSS | | GNTRPWTFGGGTKVEIK | |
| (SEQ ID NO: 312) | | (SEQ ID NO: 313) | |
| CDR Sequences (Kabat) | | CDR Sequences (Kabat) | |
| CDR-H1 | DYVIN | CDR-L1 | RASQDISNYLN |
| | (SEQ ID NO: 314) | | (SEQ ID NO: 317) |
| CDR-H2 | EIYPGSGTNYYNEKFKA | CDR-L2 | YTSRLHS |
| | (SEQ ID NO: 315) | | (SEQ ID NO: 318) |
| CDR-H3 | RGRYGLYAMDY | CDR-L3 | QQGNTRPWT |
| | (SEQ ID NO: 316) | | (SEQ ID NO: 319) |
| CDR Sequences (IMGT) | | CDR Sequences (IMGT) | |
| CDR-H1 | GYTFSDYV | CDR-L1 | QDISNY |
| | (SEQ ID NO: 320) | | (SEQ ID NO: 323) |
| CDR-H2 | IYPGSGTN | CDR-L2 | YT |
| | (SEQ ID NO: 321) | | |
| CDR-H3 | ARRGRYGLYAMDY | CDR-L3 | QQGNTRPWT |
| | (SEQ ID NO: 322) | | (SEQ ID NO: 324) |
| CDR Sequences (Chothia) | | CDR Sequences (Chothia) | |
| CDR-H1 | GYTFSDY | CDR-L1 | RASQDISNYLN |
| | (SEQ ID NO: 325) | | (SEQ ID NO: 328) |
| CDR-H2 | YPGSGT | CDR-L2 | YTSRLHS |
| | (SEQ ID NO: 326) | | (SEQ ID NO: 329) |
| CDR-H3 | RGRYGLYAMDY | CDR-L3 | QQGNTRPWT |
| | (SEQ ID NO: 327) | | (SEQ ID NO: 330) |

TABLE 2D
NKp46 Antibody Sequences (NKp46-2 H1L1)

| Heavy chain variable region | | Light chain variable region | |
|---|---|---|---|
| QVQLQESGPGLVKPSQTLSLTCTVSGYSISSD | | DIQMTQSPSSLSASVGDRVTITCRVSENIY | |
| YAWNWIRQPPGKGLEWIGYITYSGSTSYNPSL | | SYLAWYQQKPGKAPKLLVYNAKTLAEGVPS | |
| ESRVTISRDTSKNQFSLKLSSVTAADTAVYYC | | RFSGSGSGTDFTLTISSLQPEDFATYYCQH | |
| ARGGYYGSSWGVFAYWGQGTLVTVSS | | HYGTPWTFGGGTKVEIK | |
| (SEQ ID NO: 331) | | (SEQ ID NO: 332) | |
| CDR Sequences (Kabat) | | CDR Sequences (Kabat) | |
| CDR-H1 | SDYAWN | CDR-L1 | RVSENIYSYLA |
| | (SEQ ID NO: 333) | | (SEQ ID NO: 336) |
| CDR-H2 | YITYSGSTSYNPSLES | CDR-L2 | NAKTLAE |
| | (SEQ ID NO: 334) | | (SEQ ID NO: 337) |
| CDR-H3 | GGYYGSSWGVFAY | CDR-L3 | QHHYGTPWT |
| | (SEQ ID NO: 335) | | (SEQ ID NO: 338) |
| CDR Sequences (IMGT) | | CDR Sequences (IMGT) | |
| CDR-H1 | GYSISSDYA | CDR-L1 | ENIYSY |
| | (SEQ ID NO: 339) | | (SEQ ID NO: 342) |
| CDR-H2 | ITYSGST | CDR-L2 | NA |
| | (SEQ ID NO: 340) | | |
| CDR-H3 | ARGGYYGSSWGVFAY | CDR-L3 | QHHYGTPWT |
| | (SEQ ID NO: 341) | | (SEQ ID NO: 343) |
| CDR Sequences (Chothia) | | CDR Sequences (Chothia) | |
| CDR-H1 | GYSISSDY | CDR-L1 | RVSENIYSYLA |
| | (SEQ ID NO: 344) | | (SEQ ID NO: 347) |
| CDR-H2 | TYSGS | CDR-L2 | NAKTLAE |
| | (SEQ ID NO: 345) | | (SEQ ID NO: 348) |
| CDR-H3 | GGYYGSSWGVFAY | CDR-L3 | QHHYGTPWT |
| | (SEQ ID NO: 346) | | (SEQ ID NO: 349) |

TABLE 2E
NKp46 Antibody Sequences (NKp46-3 H1L1)

| Heavy chain variable region | | Light chain variable region | |
|---|---|---|---|
| QVQLVQSGAEVKKPGSSVKVSCKASGYTFSEY | | EIVMTQSPATLSVSPGERATLSCRASQSIS | |
| TMHWVRQAPGQGLEWMGGISPNIGGTSYNQKF | | DYLHWYQQKPGQAPRLLIKYASQSISGIPA | |
| KGRVTITADKSTSTAYMELSSLRSEDTAVYYC | | RFSGSGSGTDFTLTISSLEPEDFAVYYCQN | |
| ARRGGSFDYWGQGTTVTVSS | | GHSFPLTFGQGTKLEIK | |
| (SEQ ID NO: 350) | | (SEQ ID NO: 351) | |
| CDR Sequences (Kabat) | | CDR Sequences (Kabat) | |
| CDR-H1 | EYTMH | CDR-L1 | RASQSISDYLH |
| | (SEQ ID NO: 352) | | (SEQ ID NO: 355) |
| CDR-H2 | GISPNIGGTSYNQKFKG | CDR-L2 | YASQSIS |
| | (SEQ ID NO: 353) | | (SEQ ID NO: 356) |
| CDR-H3 | RGGSFDY | CDR-L3 | QNGHSFPLT |
| | (SEQ ID NO: 354) | | (SEQ ID NO: 357) |
| CDR Sequences (IMGT) | | CDR Sequences (IMGT) | |
| CDR-H1 | GYTFSEYT | CDR-L1 | QSISDY |
| | (SEQ ID NO: 358) | | (SEQ ID NO: 361) |
| CDR-H2 | ISPNIGGT | CDR-L2 | YA |
| | (SEQ ID NO: 359) | | |
| CDR-H3 | ARRGGSFDY | CDR-L3 | QNGHSFPLT |
| | (SEQ ID NO: 360) | | (SEQ ID NO: 362) |
| CDR Sequences (Chothia) | | CDR Sequences (Chothia) | |
| CDR-H1 | GYTFSEY | CDR-L1 | RASQSISDYLH |
| | (SEQ ID NO: 363) | | (SEQ ID NO: 366) |
| CDR-H2 | SPNIGG | CDR-L2 | YASQSIS |
| | (SEQ ID NO: 364) | | (SEQ ID NO: 367) |
| CDR-H3 | RGGSFDY | CDR-L3 | QNGHSFPLT |
| | (SEQ ID NO: 365) | | (SEQ ID NO: 368) |

TABLE 2F
NKp46 Antibody Sequences (NKp46-4 H1L2)

| | |
|---|---|
| Heavy chain variable region | Light chain variable region |
| QVQLVQSGAEVKKPGASVKVSCKASGYTFTSF | DIQMTQSPSSLSASVGDRVTITCRASENIY |
| TMHWVRQAPGQGLEWIGYINPSSGYTEYNQKF | SNLAWFQQKPGKAPKLLVYAATNLADGVPS |
| KDRVTITADKSTSTAYMELSSLRSEDTAVYYC | RFSGSGSGTDYTLTISSLQPEDFATYYCQH |
| VRGSSRGFDYWGQGTLVTVSS | FWGTPRTFGGGTKVEIK |
| (SEQ ID NO: 369) | (SEQ ID NO: 370) |
| CDR Sequences (Kabat) | CDR Sequences (Kabat) |
| CDR-H1 SFTMH | CDR-L1 RASENIYSNLA |
| (SEQ ID NO: 371) | (SEQ ID NO: 374) |
| CDR-H2 YINPSSGYTEYNQKFKD | CDR-L2 AATNLAD |
| (SEQ ID NO: 372) | (SEQ ID NO: 375) |
| CDR-H3 GSSRGFDY | CDR-L3 QHFWGTPRT |
| (SEQ ID NO: 373) | (SEQ ID NO: 376) |
| CDR Sequences (IMGT) | CDR Sequences (IMGT) |
| CDR-H1 GYTFTSFT | CDR-L1 ENIYSN |
| (SEQ ID NO: 377) | (SEQ ID NO: 380) |
| CDR-H2 INPSSGYT | CDR-L2 AA |
| (SEQ ID NO: 378) | CDR-L3 QHFWGTPRT |
| CDR-H3 VRGSSRGFDY | (SEQ ID NO: 381) |
| (SEQ ID NO: 379) | |
| CDR Sequences (Chothia) | CDR Sequences (Chothia) |
| CDR-H1 GYTFTSF | CDR-L1 RASENIYSNLA |
| (SEQ ID NO: 382) | (SEQ ID NO: 385) |
| CDR-H2 NPSSGY | CDR-L2 AATNLAD |
| (SEQ ID NO: 383) | (SEQ ID NO: 386) |
| CDR-H3 GSSRGFDY | CDR-L3 QHFWGTPRT |
| (SEQ ID NO: 384) | (SEQ ID NO: 387) |

TABLE 2G
NKp46 Antibody Sequences (09)

| | |
|---|---|
| Heavy chain variable region | Light chain variable region |
| EVQLQQSGAEVKKPGSSVKVSCKTSGYTFTEY | DIVMTQSPATLSLSPGERATLSCRASQSIS |
| SMHWVRQAPGKSLEWEGGISPNSGGTSYNQKF | DYLHWFQQKPHESPRLLIKYASQSISGIPA |
| KGRATLTVDKSSSTAYMELSSLRSEDTAVYYC | RFSGSGSGSDFTLTISSLEPEDFAVYYCQN |
| ARRDFHSSFDYWGQGTTVTVSS | GHSFPLTFGPGTKVDIK |
| (SEQ ID NO: 388) | (SEQ ID NO: 389) |
| CDR Sequences (Kabat) | CDR Sequences (Kabat) |
| CDR-H1 EYSMH | CDR-L1 RASQSISDYLH |
| (SEQ ID NO: 390) | (SEQ ID NO: 393) |
| CDR-H2 GISPNSGGTSYNQKFKG | CDR-L2 YASQSIS |
| (SEQ ID NO: 391) | (SEQ ID NO: 394) |
| CDR-H3 RDFHSSEDY | CDR-L3 QNGHSFPLT |
| (SEQ ID NO: 392) | (SEQ ID NO: 395) |
| CDR Sequences (IMGT) | CDR Sequences (IMGT) |
| CDR-H1 GYTFTEYS | CDR-L1 QSISDY |
| (SEQ ID NO: 396) | (SEQ ID NO: 399) |
| CDR-H2 ISPNSGGT | CDR-L2 YA |
| (SEQ ID NO: 397) | CDR-L3 QNGHSFPLT |
| CDR-H3 ARRDFHSSFDY | (SEQ ID NO: 400) |
| (SEQ ID NO: 398) | |
| CDR Sequences (Chothia) | CDR Sequences (Chothia) |
| CDR-H1 GYTFTEY | CDR-L1 RASQSISDYLH |
| (SEQ ID NO: 401) | (SEQ ID NO: 404) |
| CDR-H2 SPNSGG | CDR-L2 YASQSIS |
| (SEQ ID NO: 402) | (SEQ ID NO: 405) |
| CDR-H3 RDFHSSEDY | CDR-L3 QNGHSFPLT |
| (SEQ ID NO: 403) | (SEQ ID NO: 406) |

TABLE 3A
NKG2D Antibody Sequences (A49)

| | |
|---|---|
| Heavy chain variable region | Light chain variable region |
| EVQLVESGGGLVKPGGSLRLSCAASGFTFSSY | DIQMTQSPSSVSASVGDRVTITCRASQGIS |
| SMNWVRQAPGKGLEWVSSISSSSSYIYYADSV | SWLAWYQQKPGKAPKLLIYAASSLQSGVPS |
| KGRFTISRDNAKNSLYLQMNSLRAEDTAVYYC | RFSGSGSGTDFTLTISSLQPEDFATYYCQQ |
| ARGAPMGAAAGWFDPWGQGTLVTVSS | GVSFPRTFGGGTKVEIK |
| (SEQ ID NO: 84) | (SEQ ID NO: 85) |
| CDR Sequences (Kabat) | CDR Sequences (Kabat) |
| CDR-H1 SYSMN | CDR-L1 RASQGISSWLA |
| (SEQ ID NO: 86) | (SEQ ID NO: 89) |
| CDR-H2 SISSSSSYIYYADSVKG | CDR-L2 AASSLQS |
| (SEQ ID NO: 87) | (SEQ ID NO: 90) |
| CDR-H3 GAPMGAAAGWFDP | CDR-L3 QQGVSFPRT |
| (SEQ ID NO: 88) | (SEQ ID NO: 91) |
| CDR Sequences (IMGT) | CDR Sequences (IMGT) |
| CDR-H1 GFTFSSYS | CDR-L1 QGISSW |
| (SEQ ID NO: 92) | (SEQ ID NO: 95) |
| CDR-H2 ISSSSSYI | CDR-L2 AA |
| (SEQ ID NO: 93) | CDR-L3 QQGVSFPRT |
| CDR-H3 ARGAPMGAAAGWFDP | (SEQ ID NO: 96) |
| (SEQ ID NO: 94) | |
| CDR Sequences (Chothia) | CDR Sequences (Chothia) |
| CDR-H1 GFTFSSY | CDR-L1 RASQGISSWLA |
| (SEQ ID NO: 97) | (SEQ ID NO: 100) |
| CDR-H2 SSSSSY | CDR-L2 AASSLQS |
| (SEQ ID NO: 98) | (SEQ ID NO: 101) |
| CDR-H3 GAPMGAAAGWFDP | CDR-L3 QQGVSFPRT |
| (SEQ ID NO: 99) | (SEQ ID NO: 102) |

TABLE 3B
NKG2D Antibody Sequences (E79)

| | |
|---|---|
| Heavy chain variable region | Light chain variable region |
| QVQLVQSGAEVKKPGASVKVSCKASGYTFTSY | EIVMTQSPATLSVSPGERATLSCRASQSVS |
| YMHWVRQAPGQGLEWMGIINPSGGSTSYAQKF | SNLAWYQQKPGQAPRLLIYGASTRATGIPA |
| QGRVTMTRDTSTSTVYMELSSLRSEDTAVYYC | RFSGSGSGTEFTLTISSLQSEDFAVYYCQQ |
| ARGAPNYGDTTHDYYYMDVWGKGTTVTVSS | YDDWPFTFGGGTKVEIK |
| (SEQ ID NO: 103) | (SEQ ID NO: 104) |
| CDR Sequences (Kabat) | CDR Sequences (Kabat) |
| CDR-H1 SYYMH | CDR-L1 RASQSVSSNLA |
| (SEQ ID NO: 105) | (SEQ ID NO: 108) |
| CDR-H2 IINPSGGSTSYAQKFQG | CDR-L2 GASTRAT |
| (SEQ ID NO: 106) | (SEQ ID NO: 109) |
| CDR-H3 GAPNYGDTTHDYYYMDV | CDR-L3 QQYDDWPFT |
| (SEQ ID NO: 107) | (SEQ ID NO: 110) |
| CDR Sequences (IMGT) | CDR Sequences (IMGT) |
| CDR-H1 GYTFTSYY | CDR-L1 QSVSSN |
| (SEQ ID NO: 111) | (SEQ ID NO: 114) |
| CDR-H2 INPSGGST | CDR-L2 GA |
| (SEQ ID NO: 112) | CDR-L3 QQYDDWPFT |
| CDR-H3 ARGAPNYGDTTHDYYYMDV | (SEQ ID NO: 115) |
| (SEQ ID NO: 113) | |
| CDR Sequences (Chothia) | CDR Sequences (Chothia) |
| CDR-H1 GYTFTSY | CDR-L1 RASQSVSSNLA |
| (SEQ ID NO: 116) | (SEQ ID NO: 119) |
| CDR-H2 NPSGGS | CDR-L2 GASTRAT |
| (SEQ ID NO: 117) | (SEQ ID NO: 120) |
| CDR-H3 GAPNYGDTTHDYYYMDV | CDR-L3 QQYDDWPFT |
| (SEQ ID NO: 118) | (SEQ ID NO: 121) |

TABLE 3C
NKG2D Antibody Sequences (Tesnatilimab)

| | |
|---|---|
| Heavy chain variable region | Light chain variable region |
| QVHLQESGPGLVKPSETLSLTCTVSDDSISSY | EIVLTQSPGTLSLSPGERATLSCRASQSVS |
| YWSWIRQPPGKGLEWIGHISYSGSANYNPSLK | SSYLAWYQQKPGQAPRLLIYGASSRATGIP |
| SRVTISVDTSKNQFSLKLSSVTAADTAVYYCA | DRESGSGSGTDFTLTISRLEPEDFAVYYCQ |
| NWDDAFNIWGQGTMVTVSSA | QYGSSPWTFGQGTKVEIKR |
| (SEQ ID NO: 122) | (SEQ ID NO: 123) |
| CDR Sequences (Kabat) | CDR Sequences (Kabat) |
| CDR-H1 SYYWS | CDR-L1 RASQSVSSSYLA |
| (SEQ ID NO: 124) | (SEQ ID NO: 127) |
| CDR-H2 HISYSGSANYNPSLKS | CDR-L2 GASSRAT |
| (SEQ ID NO: 125) | (SEQ ID NO: 128) |
| CDR-H3 WDDAFNI | CDR-L3 QQYGSSPWT |
| (SEQ ID NO: 126) | (SEQ ID NO: 129) |
| CDR-H1 DDSISSYY | CDR-L1 QSVSSSY |
| (SEQ ID NO: 130) | (SEQ ID NO: 133) |
| CDR-H2 ISYSGSA | CDR-L2 GA |
| (SEQ ID NO: 131) | CDR-L3 QQYGSSPWT |
| CDR-H3 ANWDDAFNI | (SEQ ID NO: 134) |
| (SEQ ID NO: 132) | |
| CDR Sequences (Chothia) | CDR Sequences (Chothia) |
| CDR-H1 DDSISSY | CDR-L1 RASQSVSSSYLA |
| (SEQ ID NO: 135) | (SEQ ID NO: 138) |
| CDR-H2 SYSGS | CDR-L2 GASSRAT |
| (SEQ ID NO: 136) | (SEQ ID NO: 139) |
| CDR-H3 WDDAFNI | CDR-L3 QQYGSSPWT |
| (SEQ ID NO: 137) | (SEQ ID NO: 140) |

### T Cell-Binding Moieties

In some embodiments, the T cell-binding moiety is capable of binding (e.g., specifically binding) to CD3.

In some embodiments, the T cell-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which are similar to (e.g., each having at most one or two amino acids differing from) the CDRs of a CD3 antibody (e.g., a human or humanized CD3 antibody).

In some embodiments, the T cell-binding moiety is an antibody fragment comprising a heavy chain variable region (V_{H}) and light chain variable region (V_{L} or V_{K}), wherein the CDRs of the V_{H} and the V_{L} or V_{K} have sequences which are identical to those of the heavy and light chain CDRs of a CD3 antibody (e.g., a human or humanized CD3 antibody), except for one or two amino acid substitutions total across all six CDRs.

In some embodiments, the T cell-binding moiety is an antibody fragment comprising a heavy chain variable region (V_{H}) and light chain variable region (V_{L} or V_{K}), wherein the CDRs of the V_{H} and the V_{L} or V_{K} are identical to those of the heavy and light chain CDRs of a CD3 antibody (e.g., a human or humanized CD3 antibody).

In some embodiments, the T cell-binding moiety is an antibody fragment comprising heavy and light chain variable regions (e.g., V_{H} and V_{L} or V_{K}) with sequences that have at least 85%, at least 87.5%, at least 90%, at least 91%, at least 92%, at least 93%, at least 94%, at least 95%, at least 96%, at least 97%, at least 98% or at least 99% identity to those of the heavy and light chain variable regions of a CD3 antibody (e.g., a human or humanized CD3 antibody). In some embodiments, the T cell-binding moiety is an antibody fragment comprising heavy and light chain variable regions (e.g., V_{H} and V_{L} or V_{K}) with sequences identical to those of the heavy and light chain variable regions of a CD3 antibody (e.g., a human or humanized CD3 antibody).

Non-limiting examples of CD3 antibodies include otelixizumab, muromonab-CD3, teplizumab, and visilizumab. Table 4 depicts the heavy chain variable region, light chain variable region, and CDR sequences in teplizumab.

TABLE A
CD3 Antibody Sequences (Teplizumab)

| | |
|---|---|
| Heavy chain variable region | Light chain variable region |
| QVQLVQSGGGVVQPGRSLRLSCKASGYTFTRY | DIQMTQSPSSLSASVGDRVTITCSASSSVS |
| TMHWVRQAPGKGLEWIGYINPSRGYTNYNQKV | YMNWYQQTPGKAPKRWIYDTSKLASGVPSR |
| KDRFTISRDNSKNTAFLQMDSLRPEDTGVYFC | FSGSGSGTDYTFTISSLQPEDIATYYCQQW |
| ARYYDDHYCLDYWGQGTPVTVSSA | SSNPFTFGQGTKLQITRTVAAPS |
| (SEQ ID NO: 141) | (SEQ ID NO: 142) |
| CDR Sequences (Kabat) | CDR Sequences (Kabat) |
| CDR-H1 RYTMH | CDR-L1 SASSSVSYMN |
| (SEQ ID NO: 143) | (SEQ ID NO: 146) |
| CDR-H2 YINPSRGYTNYNQKVKD | CDR-L2 DTSKLAS |
| (SEQ ID NO: 144) | (SEQ ID NO: 147) |
| CDR-H3 YYDDHYCLDY | CDR-L3 QQWSSNPFT |
| (SEQ ID NO: 145) | (SEQ ID NO: 148) |
| CDR Sequences (IMGT) | CDR Sequences (IMGT) |
| CDR-H1 GYTFTRYT | CDR-L1 SSVSY |
| (SEQ ID NO: 149) | (SEQ ID NO: 152) |
| CDR-H2 INPSRGYT | CDR-L2 DT |
| (SEQ ID NO: 150) | CDR-L3 QQWSSNPFT |
| CDR-H3 ARYYDDHYCLDY | (SEQ ID NO: 153) |
| (SEQ ID NO: 151) | |
| CDR Sequences (Chothia) | CDR Sequences (Chothia) |
| CDR-H1 GYTFTRY | CDR-L1 SASSSVSYMN |
| (SEQ ID NO: 154) | (SEQ ID NO: 157) |
| CDR-H2 NPSRGY | CDR-L2 DTSKLAS |
| (SEQ ID NO: 155) | (SEQ ID NO: 158) |
| CDR-H3 YYDDHYCLDY | CDR-L3 QQWSSNPFT |
| (SEQ ID NO: 156) | (SEQ ID NO: 159) |

### Myeloid Cell-Binding Moieties

In some embodiments, the hematopoietic cell-binding moiety is a myeloid cell-binding moiety.

In some embodiments, the myeloid cell-binding moiety is capable of binding (e.g., specifically binding) to SIRPα.

In some embodiments, the myeloid cell-binding moiety is an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which are similar to (e.g., each having at most one or two amino acids differing from) the CDRs of a SIRPα antibody (e.g., a human or humanized a SIRPα antibody).

In some embodiments, the myeloid cell-binding moiety is an antibody fragment comprising a heavy chain variable region (V_{H}) and light chain variable region (V_{L} or V_{K}), wherein the CDRs of the V_{H} and the V_{L} or V_{K} have sequences which are identical to those of the heavy and light chain CDRs of a SIRPα antibody (e.g., a human or humanized SIRPα antibody), except for one or two amino acid substitutions total across all six CDRs.

In some embodiments, the myeloid cell-binding moiety is an antibody fragment comprising a heavy chain variable region (V_{H}) and light chain variable region (V_{L} or V_{K}), wherein the CDRs of the V_{H} and the V_{L} or V_{K} are identical to those of the heavy and light chain CDRs of a SIRPα antibody (e.g., a human or humanized a SIRPα antibody).

In some embodiments, the myeloid cell-binding moiety is an antibody fragment comprising heavy and light chain variable regions (e.g., V_{H} and V_{L} or V_{K}) with sequences that have at least 85%, at least 87.5%, at least 90%, at least 91%, at least 92%, at least 93%, at least 94%, at least 95%, at least 96%, at least 97%, at least 98% or at least 99% identity to those of the heavy and light chain variable regions of a SIRPα antibody (e.g., a human or humanized a SIRPα antibody). In some embodiments, the myeloid cell-binding moiety is an antibody fragment comprising heavy and light chain variable regions (e.g., V_{H} and V_{L} or V_{K}) with sequences identical to those of the heavy and light chain variable regions of a SIRPα antibody (e.g., a human or humanized a SIRPα antibody).

Non-limiting examples of SIRPα antibodies include those whose heavy chain variable region, light chain variable region, and CDR sequences are depicted in Tables 5A (1H9) and 5B (3C2).

TABLE 5A
SIRPα Antibody Sequences (1H9)

| | |
|---|---|
| Heavy chain variable region | Light chain variable region |
| QVQLVQSGAEVKKPGASVKVSCKASGYTFTSY | DIQMTQSPSSLSASVGDRVTITCRASENIY |
| WITWVKQAPGQGLEWIGDIYPGSGSTNHIEKE | SYLAWYQQKPGKAPKLLIYTAKTLAEGVPS |
| KSKATLTVDTSISTAYMELSRLRSDDTAVYYC | RFSGSGSGTDFTLTISSLQPEDFATYYCQH |
| ATGYGSSYGYFDYWGQGTLVTVSS | QYGPPFTFGQGTKLEIK |
| (SEQ ID NO: 160) | (SEQ ID NO: 161) |
| CDR Sequences (Kabat) | CDR Sequences (Kabat) |
| CDR-H1 SYWIT | CDR-L1 RASENIYSYLA |
| (SEQ ID NO: 162) | (SEQ ID NO: 165) |
| CDR-H2 DIYPGSGSTNHIEKFKS | CDR-L2 TAKTLAE |
| (SEQ ID NO: 163) | (SEQ ID NO: 166) |
| CDR-H3 GYGSSYGYFDY | CDR-L3 QHQYGPPFT |
| (SEQ ID NO: 164) | (SEQ ID NO: 167) |
| CDR Sequences (IMGT) | CDR Sequences (IMGT) |
| CDR-H1 GYTFTSYW | CDR-L1 ENIYSY |
| (SEQ ID NO: 168) | (SEQ ID NO: 171) |
| CDR-H2 IYPGSGST | CDR-L2 TA |
| (SEQ ID NO: 169) | CDR-L3 QHQYGPPFT |
| CDR-H3 ATGYGSSYGYFDY | (SEQ ID NO: 172) |
| (SEQ ID NO: 170) | |
| CDR Sequences (Chothia) | CDR Sequences (Chothia) |
| CDR-H1 GYTFTSY | CDR-L1 RASENIYSYLA |
| (SEQ ID NO: 173) | (SEQ ID NO: 176) |
| CDR-H2 YPGSGS | CDR-L2 TAKTLAE |
| (SEQ ID NO: 174) | (SEQ ID NO: 177) |
| CDR-H3 GYGSSYGYFDY | CDR-L3 QHQYGPPFT |
| (SEQ ID NO: 175) | (SEQ ID NO: 178) |

TABLE 5B
SIRPα Antibody Sequences (3C2)

| | |
|---|---|
| HCVR | LCVR |
| QVQLVQSGAEVKKPGASVKVSCKASGYTFTSY | DIVMTQTPLSLSVTPGQPASISCRSSQSIV |
| WMHWVRQAPGQGLEWMGNIDPSDSDTHYNQKF | HSYGNTYLEWYLQKPGQSPQLLIYKVSNRF |
| KDRVTMTRDTSTSTVYMELSSLRSEDTAVYYC | SGVPDRFSGSGSGTDFTLKISRVEAEDVGV |
| ARGYSKYYAMDYWGQGTLVTVSS | YYCFQGSHVPYTFGQGTKLEIK |
| (SEQ ID NO: 179) | (SEQ ID NO: 180) |
| CDR Sequences (Kabat) | CDR Sequences (Kabat) |
| CDR-H1 SYWMH | CDR-L1 RSSQSIVHSYGNTYLE |
| (SEQ ID NO: 181) | (SEQ ID NO: 184) |
| CDR-H2 NIDPSDSDTHYNQKFKD | CDR-L2 KVSNRFS |
| (SEQ ID NO: 182) | (SEQ ID NO: 185) |
| CDR-H3 GYSKYYAMDY | CDR-L3 FQGSHVPYT |
| (SEQ ID NO: 183) | (SEQ ID NO: 186) |
| CDR Sequences (IMGT) | CDR Sequences (IMGT) |
| CDR-H1 GYTFTSYW | CDR-L1 QSIVHSYGNTY |
| (SEQ ID NO: 187) | (SEQ ID NO: 190) |
| CDR-H2 IDPSDSDT | CDR-L2 KV |
| (SEQ ID NO: 188) | CDR-L3 FQGSHVPYT |
| CDR-H3 ARGYSKYYAMDY | (SEQ ID NO: 191) |
| (SEQ ID NO: 189) | |
| CDR Sequences (Chothia) | CDR Sequences (Chothia) |
| CDR-H1 GYTFTSY | CDR-L1 RSSQSIVHSYGNTYLE |
| (SEQ ID NO: 192) | (SEQ ID NO: 195) |
| CDR-H2 DPSDSD | CDR-L2 KVSNRFS |
| (SEQ ID NO: 193) | (SEQ ID NO: 196) |
| CDR-H3 GYSKYYAMDY | CDR-L3 FQGSHVPYT |
| (SEQ ID NO: 194) | (SEQ ID NO: 197) |

### B. Tumor-Binding Moieties

Tumor-binding moieties are generally capable of binding (e.g., specifically binding) to a tumor-associated antigen (e.g., a tumor-specific antigen) expressed on a tumor cell or a cell that supports a tumor (e.g., a stromal cell.) In some embodiments, the tumor-binding moiety is capable of binding (e.g., specifically binding) to CD20 or CD37.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which are similar to (e.g., each having at most one or two amino acids differing from) the CDRs of a CD20 or CD37 antibody (e.g., a human or humanized CD20 or CD37 antibody).

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} have sequences which are identical to those of the heavy and light chain CDRs of a CD20 or CD37 antibody (e.g., a human or humanized CD20 or CD37 antibody), except for one or two amino acid substitutions total across all six CDRs.

In some embodiments, the tumor-binding moiety comprises an antibody fragment comprising a heavy chain variable region (V_{H}) and light chain variable region (V_{L} or V_{K}), wherein the CDRs of the V_{H} and the V_{L} or V_{K} are identical to those of the heavy and light chain CDRs of a CD20 or CD37 antibody (e.g., a human or humanized CD20 or CD37 antibody).

In some embodiments, the tumor-binding moiety comprises an antibody fragment comprising heavy and light chain variable regions (e.g., V_{H} and V_{L} or V_{K}) with sequences that have at least 85%, at least 87.5%, at least 90%, at least 91%, at least 92%, at least 93%, at least 94%, at least 95%, at least 96%, at least 97%, at least 98% or at least 99% identity to those of the heavy and light chain variable regions of a CD20 or CD37 antibody, e.g., a human or humanized CD20 or CD37 antibody. In some embodiments, the tumor-binding moiety is an antibody fragment comprising heavy and light chain variable regions (e.g., V_{H} and V_{L} or V_{K}) with sequences identical to those of the heavy and light chain variable regions of a CD20 or CD37 antibody (e.g., a human or humanized CD20 or CD37 antibody).

Non-limiting examples of CD20 antibodies include rituximab, ofatumumab, veltuzumab, and ocrelizumab. Tables 6A and 6B depict the heavy chain variable region, light chain variable region, and CDR sequences of rituximab and ofatumumab, respectively.

Non-limiting examples of CD37 antibodies include naratuximab, lilotomab, AGS67C, and otlertuzumab. Tables 7A, 7B, 7C, and 7D depict the heavy chain variable region, light chain variable region, and CDR sequences of naratuximab, lilotomab, AGS67C, and otlertuzumab respectively.

TABLE 6A
CD20 Antibody Sequences (Rituxiumab)

| | |
|---|---|
| Heavy chain variable region | Light chain variable region |
| QVQLQQPGAELVKPGASVKMSCKASGYTFTSY | QIVLSQSPAILSASPGEKVTMTCRASSSVS |
| NMHWVKQTPGRGLEWIGAIYPGNGDTSYNQKF | YIHWFQQKPGSSPKPWIYATSNLASGVPVR |
| KGKATLTADKSSSTAYMQLSSLTSEDSAVYYC | FSGSGSGTSYSLTISRVEAEDAATYYCQQW |
| ARSTYYGGDWYFNVWGAGTTVTVSAA | TSNPPTFGGGTKLEIKR |
| (SEQ ID NO: 198) | (SEQ ID NO: 199) |
| CDR Sequences (Kabat) | CDR Sequences (Kabat) |
| CDR-H1 SYNMH | CDR-L1 RASSSVSYIH |
| (SEQ ID NO: 200) | (SEQ ID NO: 203) |
| CDR-H2 AIYPGNGDTSYNQKFKG | CDR-L2 ATSNLAS |
| (SEQ ID NO: 201) | (SEQ ID NO: 204) |
| CDR-H3 STYYGGDWYFNV | CDR-L3 QQWTSNPPT |
| (SEQ ID NO: 202) | (SEQ ID NO: 205) |
| CDR Sequences (IMGT) | CDR Sequences (IMGT) |
| CDR-H1 GYTFTSYN | CDR-L1 SSVSY |
| (SEQ ID NO: 206) | (SEQ ID NO: 209) |
| CDR-H2 IYPGNGDT | CDR-L2 AT |
| (SEQ ID NO: 207) | CDR-L3 QQWTSNPPT |
| CDR-H3 ARSTYYGGDWYFNV | (SEQ ID NO: 210) |
| (SEQ ID NO: 208) | |
| CDR Sequences (Chothia) | CDR Sequences (Chothia) |
| CDR-H1 GYTFTSY | CDR-L1 RASSSVSYIH |
| (SEQ ID NO: 211) | (SEQ ID NO: 214) |
| CDR-H2 YPGNGD | CDR-L2 ATSNLAS |
| (SEQ ID NO: 212) | (SEQ ID NO: 215) |
| CDR-H3 STYYGGDWYFNV | CDR-L3 QQWTSNPPT |
| (SEQ ID NO: 213) | (SEQ ID NO: 216) |

TABLE 6B
CD20 Antibody Sequences (Ofatumumab)

| | |
|---|---|
| Heavy chain variable region | Light chain variable region |
| EVQLVESGGGLVQPGRSLRLSCAASGFTENDY | EIVLTQSPATLSLSPGERATLSCRASQSVS |
| AMHWVRQAPGKGLEWVSTISWNSGSIGYADSV | SYLAWYQQKPGQAPRLLIYDASNRATGIPA |
| KGRFTISRDNAKKSLYLQMNSLRAEDTALYYC | RFSGSGSGTDFTLTISSLEPEDFAVYYCQQ |
| AKDIQYGNYYYGMDVWGQGTTVTVSSA | RSNWPITFGQGTRLEIKR |
| (SEQ ID NO: 217) | (SEQ ID NO: 218) |
| CDR Sequences (Kabat) | CDR Sequences (Kabat) |
| CDR-H1 DYAMH | CDR-L1 RASQSVSSYLA |
| (SEQ ID NO: 219) | (SEQ ID NO: 222) |
| CDR-H2 TISWNSGSIGYADSVKG | CDR-L2 DASNRAT |
| (SEQ ID NO: 220) | (SEQ ID NO: 223) |
| CDR-H3 DIQYGNYYYGMDV | CDR-L3 QQRSNWPIT |
| (SEQ ID NO: 221) | (SEQ ID NO: 224) |
| CDR Sequences (IMGT) | CDR Sequences (IMGT) |
| CDR-H1 GFTENDYA | CDR-L1 QSVSSY |
| (SEQ ID NO: 225) | (SEQ ID NO: 228) |
| CDR-H2 ISWNSGSI | CDR-L2 DA |
| (SEQ ID NO: 226) | CDR-L3 QQRSNWPIT |
| CDR-H3 AKDIQYGNYYYGMDV | (SEQ ID NO: 229) |
| (SEQ ID NO: 227) | |
| CDR Sequences (Chothia) | CDR Sequences (Chothia) |
| CDR-H1 GFTENDY | CDR-L1 RASQSVSSYLA |
| (SEQ ID NO: 230) | (SEQ ID NO: 233) |
| CDR-H2 SWNSGS | CDR-L2 DASNRAT |
| (SEQ ID NO: 231) | (SEQ ID NO: 234) |
| CDR-H3 DIQYGNYYYGMDV | CDR-L3 QQRSNWPIT |
| (SEQ ID NO: 232) | (SEQ ID NO: 235) |

TABLE 7A
CD37 Antibody Sequences (Naratuximab)

| | |
|---|---|
| Heavy chain variable region | Light chain variable region |
| QVQVQESGPGLVAPSQTLSITCTVSGESLTTS | DIQMTQSPSSLSVSVGERVTITCRASENIR |
| GVSWVRQPPGKGLEWLGVIWGDGSTNYHPSLK | SNLAWYQQKPGKSPKLLVNVATNLADGVPS |
| SRLSIKKDHSKSQVFLKLNSLTAADTATYYCA | RFSGSGSGTDYSLKINSLQPEDFGTYYCQH |
| KGGYSLAHWGQGTLVTVSSA | YWGTTWTFGQGTKLEIKR |
| (SEQ ID NO: 236) | (SEQ ID NO: 237) |
| CDR Sequences (Kabat) | CDR Sequences (Kabat) |
| CDR-H1 TSGVS | CDR-L1 RASENIRSNLA |
| (SEQ ID NO: 238) | (SEQ ID NO: 241) |
| CDR-H2 VIWGDGSTNYHPSLKS | CDR-L2 VATNLAD |
| (SEQ ID NO: 239) | (SEQ ID NO: 242) |
| CDR-H3 GGYSLAH | CDR-L3 QHYWGTTWT |
| (SEQ ID NO: 240) | (SEQ ID NO: 243) |
| CDR Sequences (IMGT) | CDR Sequences (IMGT) |
| CDR-H1 GFSLTTSG | CDR-L1 ENIRSN |
| (SEQ ID NO: 244) | (SEQ ID NO: 247) |
| CDR-H2 IWGDGST | CDR-L2 VA |
| (SEQ ID NO: 245) | CDR-L3 QHYWGTTWT |
| CDR-H3 AKGGYSLAH | (SEQ ID NO: 248) |
| (SEQ ID NO: 246) | |
| CDR Sequences (Chothia) | CDR Sequences (Chothia) |
| CDR-H1 GFSLTTS | CDR-L1 RASENIRSNLA |
| (SEQ ID NO: 249) | (SEQ ID NO: 252) |
| CDR-H2 WGDGS | CDR-L2 VATNLAD |
| (SEQ ID NO: 250) | (SEQ ID NO: 253) |
| CDR-H3 GGYSLAH | CDR-L3 QHYWGTTWT |
| (SEQ ID NO: 251) | (SEQ ID NO: 254) |

TABLE 7B
CD37 Antibody Sequences (Lilotomab)

| | |
|---|---|
| Heavy chain variable region | Light chain variable region |
| EIQLQQSGPELVKPGASVKVSCKASGYSFTDY | DIVMTQSHKLLSTSVGDRVSITCKASQDVS |
| NMYWVKQSHGKSLEWIGYIDPYNGDTTYNQKE | TAVDWYQQKPGQSPKLLINWASTRHTGVPD |
| KGKATLTVDKSSSTAFIHLNSLTSEDSAVYYC | RFTGSGSGTDYTLTISSMQAEDLALYYCRQ |
| ARS PYGHYAMDYWGQGTSVTVSS | HYSTPFTFGSGTKLEIKR |
| (SEQ ID NO: 255) | (SEQ ID NO: 256) |
| CDR Sequences (Kabat) | CDR Sequences (Kabat) |
| CDR-H1 DYNMY | CDR-L1 KASQDVSTAVD |
| (SEQ ID NO: 257) | (SEQ ID NO: 260) |
| CDR-H2 YIDPYNGDTTYNQKFKG | CDR-L2 WASTRHT |
| (SEQ ID NO: 258) | (SEQ ID NO: 261) |
| CDR-H3 SPYGHYAMDY | CDR-L3 RQHYSTPFT |
| (SEQ ID NO: 259) | (SEQ ID NO: 262) |
| CDR Sequences (IMGT) | CDR Sequences (IMGT) |
| CDR-H1 GYSFTDYN | CDR-L1 QDVSTA |
| (SEQ ID NO: 263) | (SEQ ID NO: 266) |
| CDR-H2 IDPYNGDT | CDR-L2 WA |
| (SEQ ID NO: 264) | CDR-L3 RQHYSTPFT |
| CDR-H3 ARSPYGHYAMDY | (SEQ ID NO: 267) |
| (SEQ ID NO: 265) | |
| CDR Sequences (Chothia) | CDR Sequences (Chothia) |
| CDR-H1 GYSFTDY | CDR-L1 KASQDVSTAVD |
| (SEQ ID NO: 268) | (SEQ ID NO: 271) |
| CDR-H2 DPYNGD | CDR-L2 WASTRHT |
| (SEQ ID NO: 269) | (SEQ ID NO: 272) |
| CDR-H3 SPYGHYAMDY | CDR-L3 RQHYSTPFT |
| (SEQ ID NO: 270) | (SEQ ID NO: 273) |

TABLE 7C
CD37 Antibody Sequences (AGS67C)

| | |
|---|---|
| Heavy chain variable region | Light chain variable region |
| QVQLQQWGAGLLKPSETLSLTCAVYGGSESPY | DIQMTQSPSTLSASVGDRVTITCRASQSIS |
| YWSWIRQPPGKGLEWIGEINHSGSTNYNPSLK | SWLAWYQQKPGKAPKLLIYKASSLESGVPS |
| SRVTISVDTSKNQFSLKLSSVTAADTAVYYCA | RFSGSGSGTEFTLTISSLQPDDFATYYCQQ |
| RRAGDFDYWGQGTLVTVSS | YNSYIFGQGTKLEIK |
| (SEQ ID NO: 274) | (SEQ ID NO: 275) |
| CDR Sequences (Kabat) | CDR Sequences (Kabat) |
| CDR-H1 PYYWS | CDR-L1 RASQSISSWLA |
| (SEQ ID NO: 276) | (SEQ ID NO: 279) |
| CDR-H2 EINHSGSTNYNPSLKS | CDR-L2 KASSLES |
| (SEQ ID NO: 277) | (SEQ ID NO: 280) |
| CDR-H3 RAGDEDY | CDR-L3 QQYNSYI |
| (SEQ ID NO: 278) | (SEQ ID NO: 281) |
| CDR Sequences (IMGT) | CDR Sequences (IMGT) |
| CDR-H1 GGSFSPYY | CDR-L1 QSISSW |
| (SEQ ID NO: 282) | (SEQ ID NO: 285) |
| CDR-H2 INHSGST | CDR-L2 KA |
| (SEQ ID NO: 283) | CDR-L3 QQYNSYI |
| CDR-H3 ARRAGDEDY | (SEQ ID NO: 286) |
| (SEQ ID NO: 284) | |
| CDR Sequences (Chothia) | CDR Sequences (Chothia) |
| CDR-H1 GGSFSPY | CDR-L1 RASQSISSWLA |
| (SEQ ID NO: 287) | (SEQ ID NO: 290) |
| CDR-H2 NHSGS | CDR-L2 KASSLES |
| (SEQ ID NO: 288) | (SEQ ID NO: 291) |
| CDR-H3 RAGDEDY | CDR-L3 QQYNSYI |
| (SEQ ID NO: 289) | (SEQ ID NO: 292) |

TABLE 7D
CD37 Antibody Sequences (Otlertuzumab)

| | |
|---|---|
| Heavy chain variable region | Light chain variable region |
| EVQLVQSGAEVKKPGESLKISCKGSGYSFTGY | EIVLTQSPATLSLSPGERATLSCRASENVY |
| NMNWVRQMPGKGLEWMGNIDPYYGGTTYNRKF | SYLAWYQQKPGQAPRLLIYFAKTLAEGIPA |
| KGQVTISADKSISTAYLQWSSLKASDTAMYYC | RFSGSGSGTDFTLTISSLEPEDFAVYYCQH |
| ARSVGPFDSWGQGTLVTVSSG | HSDN PWT FGQGTKVEIK |
| (SEQ ID NO: 293) | (SEQ ID NO: 294) |
| CDR Sequences (Kabat) | CDR Sequences (Kabat) |
| CDR-H1 GYNMN | CDR-L1 RASENVYSYLA |
| (SEQ ID NO: 295) | (SEQ ID NO: 298) |
| CDR-H2 NIDPYYGGTTYNRKFKG | CDR-L2 FAKTLAE |
| (SEQ ID NO: 296) | (SEQ ID NO: 299) |
| CDR-H3 SVGPFDS | CDR-L3 QHHSDNPWT |
| (SEQ ID NO: 297) | (SEQ ID NO: 300) |
| CDR Sequences (IMGT) | CDR Sequences (IMGT) |
| CDR-H1 GYSFTGYN | CDR-L1 ENVYSY |
| (SEQ ID NO: 301) | (SEQ ID NO: 304) |
| CDR-H2 IDPYYGGT | CDR-L2 FA |
| (SEQ ID NO: 302) | CDR-L3 QHHSDNPWT |
| CDR-H3 ARSVGPFDS | (SEQ ID NO: 305) |
| (SEQ ID NO: 303) | |
| CDR Sequences (Chothia) | CDR Sequences (Chothia) |
| CDR-H1 GYSFTGY | CDR-L1 RASENVYSYLA |
| (SEQ ID NO: 306) | (SEQ ID NO: 309) |
| CDR-H2 DPYYGG | CDR-L2 FAKTLAE |
| (SEQ ID NO: 307) | (SEQ ID NO: 310) |
| CDR-H3 SVGPFDS | CDR-L3 QHHSDNPWT |
| (SEQ ID NO: 308) | (SEQ ID NO: 311) |

TABLE 7E
CD37 Antibody Sequences (016-H5L2)

| | |
|---|---|
| Heavy chain variable region | Light chain variable region |
| EVQLVESGGGLVQPGGSLRLSCAASGESLSNY | DVVMTQSPSTLSASVGDRVTITCQASQNID |
| NMGWVRQAPGKGLEWVSVIDASGTTYYATWAK | SNLAWYQQKPGKAPKFLIYYASNLPFGVPS |
| GRFTISRDNSKNTLYLQMNSLRAEDTATYYCA | RFKGSGSGTEFTLTISSLQPDDFATYYCQS |
| RELLYFGSSYYDLWGQGTLVTVSS | ADVGSTYVAAFGGGTKVEIK |
| (SEQ ID NO: 407) | (SEQ ID NO: 408) |
| CDR Sequences (Kabat) | CDR Sequences (Kabat) |
| CDR-H1 NYNMG | CDR-L1 QASQNIDSNLA |
| (SEQ ID NO: 409) | (SEQ ID NO: 412) |
| CDR-H2 VIDASGTTYYATWAKG | CDR-L2 YASNLPE |
| (SEQ ID NO: 410) | (SEQ ID NO: 413) |
| CDR-H3 ELLYFGSSYYDL | CDR-L3 QSADVGSTYVAA |
| (SEQ ID NO: 411) | (SEQ ID NO: 414) |
| CDR Sequences (IMGT) | CDR Sequences (IMGT) |
| CDR-H1 GFSLSNYN | CDR-L1 QNIDSN |
| (SEQ ID NO: 415) | (SEQ ID NO: 418) |
| CDR-H2 IDASGTT | CDR-L2 YA |
| (SEQ ID NO: 416) | CDR-L3 QSADVGSTYVAA |
| CDR-H3 ARELLYFGSSYYDL | (SEQ ID NO: 419) |
| (SEQ ID NO: 417) | |
| CDR Sequences (Chothia) | CDR Sequences (Chothia) |
| CDR-H1 GFSLSNY | CDR-L1 QASQNIDSNLA |
| (SEQ ID NO: 420) | (SEQ ID NO: 423) |
| CDR-H2 DASGT | CDR-L2 YASNLPF |
| (SEQ ID NO: 421) | (SEQ ID NO: 424) |
| CDR-H3 ELLYFGSSYYDL | CDR-L3 QSADVGSTYVAA |
| (SEQ ID NO: 422) | (SEQ ID NO: 425) |

### 3. Fc Polypeptides

In some embodiments, the Fc polypeptide is a single chain Fc (scFc), which comprises two Fc chains linked together by a covalent linker, e.g., via an amino acid linker.

An IgG Fc chain (e.g., IgG1 Fc chain) typically contains two constant heavy domains (CH2 and CH3) and a hinge region connected to the CH2 domain.

In some embodiments, the Fc polypeptide comprises one or more IgG1 Fc chains (e.g., human IgG1 Fc chains or a human Fc chains), that is, the Fc polypeptide comprises an Fc chain that has an amino acid sequence that is substantially similar or identical to that of the chains within a wild type IgG1 Fc.

In some embodiments, the Fc polypeptide comprises one or more human IgG1 Fc chains; that is, the Fc polypeptide comprises an Fc chain that is substantially similar or identical to that of the Fc chains within a wild type human IgG1. In some embodiments, the wild type human IgG1 Fc has an amino acid sequence of SEQ ID NO:4.

In some embodiments, the Fc chain comprise one or more IgG1, IgG2, IgG3, or IgG4 heavy chain constant regions, e.g., one or more heavy chain constant region having at least 90%, at least 91%, at least 92%, at least 93%, at least 94%, at least 95%, at least 96%, at least 97%, at least 98%, at least 99%, or identical to that of SEQ ID NO:426, 427, 428, or 429.

### 4. Linkers

In certain embodiments, linkers are used within fusion polypeptides and/or within single-chain molecules such as scFcs. In some embodiments, the linker is an amino acid linker. For example, a linker as employed herein may comprise from about 1 to about 100 amino acid residues, e.g., about 1 to about 70, about 2 to about 70, about 1 to about 30, or about 2 to about 30 amino acid residues. In some embodiments, the linker comprises at least 2, at least 3, at least 4, at least 5, at least 6, at least 7, at least 8, at least 9, or at least 10 amino acid residues.

In certain embodiments, the linker comprises a glycine-serine sequence, e.g., a (GₙS)ₘ sequence (e.g., GGS, GGGS (SEQ ID NO: 6), and/or GGGGS (SEQ ID NO: 7) sequence) that is present in at least 1, at least 2, at least 3, at least 4, at least 5, at least 6, at least 7, at least 8, at least 9, at least 10, at least 11, at least 12, at least 13, or at least 14 copies within the linker.

### B. Self-Assembled Polypeptide Complexes

In one aspect, provided are self-assembled polypeptide complexes (“Multabodies” or “MBs”) comprising a plurality of fusion polypeptides as disclosed herein. Generally, provided self-assembled polypeptide complexes comprise (a) a plurality of hematopoietic cell-binding fusion polypeptides, each hematopoietic cell-binding fusion polypeptide comprising (1) a hematopoietic cell-binding moiety linked to (2) a nanocage monomer or subunit thereof, and (b) a plurality of Fc fusion polypeptides, each Fc fusion polypeptide comprising (1) an Fc polypeptide linked to (2) a nanocage monomer or subunit thereof, wherein the Fc polypeptide comprises an Fc chain.

In some embodiments, the self-assembled polypeptide complexes further comprise a plurality of tumor-binding fusion polypeptides, each tumor-binding fusion polypeptide comprising (1) a tumor-binding moiety linked to (2) a nanocage monomer or subunit thereof.

In some embodiments, the nanocage monomer is a ferritin monomer, and each fusion polypeptide within the self-assembled polypeptide complex comprises a ferritin light chain or a subunit of a ferritin light chain. In these embodiments, the self-assembled polypeptide complex does not comprise any ferritin heavy chains, subunits of ferritin heavy chains, or other ferritin components capable of binding to iron or capable of ferroxidase activity.

In some embodiments, the nanocage monomer or subunit thereof is a ferritin monomer subunit, and (a) each hematopoietic cell-binding fusion polypeptide comprises a ferritin monomer subunit which is C-half-ferritin and each Fc fusion polypeptide comprises a ferritin monomer subunit which is N-half-ferritin; or (b) each hematopoietic cell-binding fusion polypeptide comprises a ferritin monomer subunit which is N-half ferritin and each Fc fusion polypeptide comprises a ferritin monomer subunit which is C-half-ferritin.

In some embodiments, the self-assembled polypeptide complex comprises between 24 and 48 fusion polypeptides in total. In some embodiments, the self-assembled polypeptide complex comprises 24 fusion polypeptides in total. In some embodiments, the self-assembled polypeptide complex comprises more than 24 fusion polypeptides, e.g., at least 26, at least 28, at least 30, at least 32 fusion polypeptides, at least 34 fusion polypeptides, at least 36 fusion polypeptides, at least 38 fusion polypeptides, at least 40 fusion polypeptides, at least 42 fusion polypeptides, at least 44 fusion polypeptides, at least 46 fusion polypeptides, or at least 48 fusion polypeptides in total. In some embodiments, the self-assembled polypeptide complex comprises about 32 fusion polypeptides.

In some embodiments, the self-assembled polypeptide complex comprises at least 4, at least 5, least 6, at least 7, or at least 8 hematopoietic cell-binding fusion polypeptides.

In some embodiments, the self-assembled polypeptide complex comprises at least 4, at least 5, least 6, at least 7, or at least 8 Fc fusion polypeptides.

In some embodiments, the self-assembled polypeptide complex further comprises at least 4, at least 5, least 6, at least 7, at least 8, at least 9, at least 10, least 11, at least 12, at least 13, at least 14, at least 15, or at least 16 third fusion polypeptides.

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of hematopoietic cell-binding fusion polypeptides to all other fusion polypeptides.

In some embodiments, the self-assembled polypeptide complex comprises a ratio of approximately 1:1, 11:13, 3:5, 1:2, 7:17, 1:3, 2:7, 5:19, 1:4, 1:5, 1:6, 1:7, 1:8, 1:12, 1:24 of hematopoietic cell-binding fusion polypeptides to Fc fusion polypeptides.

### Pharmacokinetic Characteristics

In certain embodiments, when administered to a subject in need thereof, a provided self-assembled polypeptide complex, has one or more pharmacokinetic features similar to that of a reference IgG molecule (e.g., an IgG molecule whose class matches the class of an Fc chain within an Fc polypeptide of an Fc fusion polypeptide within the self-assembled polypeptide complex).

### Effects

In certain embodiments, a provided self-assembled polypeptide complex is capable of inducing antibody-dependent cellular cytotoxicity (ADCC), e.g., to kill a target cell (e.g., tumor cell) when contacted with a target cell.

ADCC can be assed in vitro using any of a number of assays, e.g., a cell-based assay employing an effector cell (such as an NK cell, peripheral blood mononuclear cell), a target cell (such as a tumor cell), and a reporter which indicates target cell lysis and/or activation within the effector cell.

### C. Methods of Treatment and Methods of Contacting Tumor Cells

In one aspect, provided are methods that may be useful for treating, ameliorating, or preventing a disease or a condition, generally comprising a step of administering a self-assembled polypeptide complex of the present disclosure (or a composition thereof) to a subject.

In some embodiments, the subject is a mammal, e.g., a human.

Compositions for administration to subjects generally comprise a self-assembled polypeptide complex as disclosed herein. In some embodiments, such compositions further comprise a pharmaceutically acceptable excipient.

Compositions may be formulated for administration for any of a variety of routes of administration, including systemic routes (e.g., oral, inhalation, intranasal, intravenous, intraperitoneal, subcutaneous, or intramuscular administration).

In some embodiments, the step of administering results in improvement in one or more clinical outcomes or metrics in the subject.

For example, in some embodiments, the subject has cancer, e.g., a B-cell lymphoma.

In some embodiments, the step of administering results in slowing or inhibiting progression of the tumor, e.g., regression of the tumor. In some embodiments, the step of administering results in complete regression of the tumor.

### Methods of Contacting

The present disclosure also provides methods of contacting self-assembled polypeptide complexes as disclosed herein with a tumor cell. In some embodiments, the step of contacting occurs inside a mammalian body, e.g., a human body.

In some embodiments, the step of contacting induces apoptosis of the tumor cell. In some embodiments, apoptosis is induced with an EC₅₀ value (for the self-assembled polypeptide complex) of less than 100 nM, less than 50 nM, less than 10 nM, less than 5 nM, less than 1 nM, less than 0.5 nM, less than 0.1 nM, or less than 0.05 nM. In some embodiments, the tumor cell expresses CD37, and the self-assembled polypeptide complex comprises a fusion protein comprising an α-CD37 Fab.

In some embodiments, the step of contacting is conducted in the presence of hematopoietic cells, e.g., NK cells, and induces antibody-dependent cellular cytotoxicity (ADCC) of the tumor cell. In some embodiments, ADCC is induced with an EC50 value (for the self-assembled polypeptide complex) of less than 1 nM, less than 500 pM, less than 100 pM, less than 50 pM, less than 10 pM, less than 5 pM, less than 4 pM, less than 3 pM, less than 2 pM, less than 1 pM, and less than 0.5 pM.

In some embodiments, the step of contacting is conducted in the presence of complement proteins and results in complement-dependent cytotoxicity (CDC) of the tumor cell. In some embodiments, CDC is induced with an EC50 value (for the self-assembled polypeptide complex) of less than 10 nM, less than 1.0 nM, less than 0.50 nM, less than 0.40 nM, less than 0.30 nM, less than 0.20 nM, or less than 0.15 nM.

In some embodiments, the step of contacting is conducted in the presence of hematopoietic cells, e.g., myeloid cells, and results in antibody-dependent cellular phagocytosis (ADCP) of the tumor cell. In some embodiments, ADCP is induced with an EC₅₀ value (for the self-assembled polypeptide complex) of less than 100 pM (0.1 nM), less than 50 pM, less than 25 pM, less than 20 pM, less than 18 pM, less than 15 pM, or less than 10 pM.

### EXAMPLES

### Example 1. Construction and Expression of Immune Cell-Engaging or Activating Multabodies (MBs)

This Example describes the generation of immune cell-engaging or activating Multabodies (MBs) which self-assemble from pluralities of at least two types of ferritin-containing fusion polypeptides, specifically, MBs which comprise:
(1) One or both of:
   (a) a tumor-binding fusion polypeptide, each comprising human ferritin light chain or a subunit thereof and an antibody fragment that is capable of binding (e.g., specifically binding) to a tumor cell; and
   (b) an immune-cell binding fusion polypeptide comprising human ferritin light chain or a subunit thereof and an antibody fragment that is capable of binding (e.g., specifically binding) to an immune cell; and
(2) an Fc fusion polypeptide comprising human ferritin light chain or a subunit thereof and an Fc polypeptide.

Sets of constructs encoding the following fusion proteins are prepared, mixed at a predefined molar ratio, and then transiently transfected into cells, which express the encoded polypeptides. The following are non-limiting examples of sets of fusion polypeptides that can be used to generate MBs that engage or activate an immune cell.

### Set 1

(1) single-chain Fab (scFab) of an antibody that is capable of binding (e.g., specifically binding) to an immune cell fused to the light chain of human ferritin via a linker; and
(2) single-chain Fc (scFc) fused to the light chain of human ferritin via a linker.

### Set 2

(1) scFab of an antibody that is capable of binding (e.g., specifically binding) to an immune cell fused to the N-half of a light chain of human ferritin via a linker
(2) scFab of an antibody that is capable of binding (e.g., specifically binding) to a tumor-associated antigen fused to the C-half of a light chain of human ferritin via a linker, and
(3) scFc fused to a light chain of human ferritin via a linker,
wherein the ratio of polypeptides (1) and (2) is 1:1.

### Set 3

(1) scFab of an antibody that is capable of binding (e.g., specifically binding) to an immune cell fused to the C-half of a light chain of human ferritin via a linker
(2) scFab of an antibody that is capable of binding (e.g., specifically binding) to a tumor-associated antigen fused to the N-half of a light chain of human ferritin via a linker, and
(3) scFc fused to a light chain of human ferritin via a linker,
wherein the ratio of polypeptides (1) and (2) is 1:1.

### Set 4

(1) scFab of an antibody that is capable of binding (e.g., specifically binding) to an immune cell fused to the light chain of human ferritin via a linker;
(2) scFab of an antibody that is capable of binding (e.g., specifically binding) to a tumor-associated antigen fused to the N-half of a light chain of human ferritin via a linker; and
(3) scFc fused to a C-half of a light chain of human ferritin via a linker,
wherein the ratio of polypeptides (2) and (3) is 1:1.

### Set 5

(1) scFab of an antibody that is capable of binding (e.g., specifically binding) to an immune cell fused to the light chain of human ferritin via a linker;
(2) scFab of an antibody that is capable of binding (e.g., specifically binding) to a tumor-associated antigen fused to the C-half of a light chain of human ferritin via a linker; and
(3) scFc fused to a N-half of a light chain of human ferritin via a linker,
wherein the ratio of polypeptides (2) and (3) is 1:1.

### Set 6

(1) scFab of an antibody that is capable of binding (e.g., specifically binding) to an immune cell fused to the N-half of a light chain of human ferritin via a linker;
(2) scFab of an antibody that is capable of binding (e.g., specifically binding) to a tumor-associated antigen fused to a light chain of human ferritin via a linker; and
(3) scFc fused to a C-half of a light chain of human ferritin via a linker,
wherein the ratio of polypeptides (2) and (3) is 1:1.

### Set 7

(1) scFab of an antibody that is capable of binding (e.g., specifically binding) to an immune cell fused to the C-half of a light chain of human ferritin via a linker;
(2) scFab of an antibody that is capable of binding (e.g., specifically binding) to a tumor-associated antigen fused to a light chain of human ferritin via a linker; and
(3) scFc fused to a N-half of a light chain of human ferritin via a linker,
wherein the ratio of polypeptides (1) and (3) is 1:1.

### Set 8

(1) scFab of an antibody that is capable of binding (e.g., specifically binding) to a tumor-associated antigen fused to the light chain of human ferritin via a linker; and
(2) scFc fused to the light chain of human ferritin via a linker.

### Set 9

(1) scFab of an antibody that is capable of binding (e.g., specifically binding) to a tumor-associated antigen fused to the C-half of a light chain of human ferritin via a linker, and
(2) scFc fused to the N-half of light chain of human ferritin via a linker,
wherein the ratio of polypeptides (1) and (2) is 1:1.

### Set 10

(1) scFab of an antibody that is capable of binding (e.g., specifically binding) to a tumor-associated antigen fused to the N-half of a light chain of human ferritin via a linker, and
(2) scFc fused to the C-half of light chain of human ferritin via a linker,
wherein the ratio of polypeptides (1) and (2) is 1:1.

### Set 11

(1) scFab of an antibody that is capable of binding (e.g., specifically binding) to a tumor-associated antigen fused to the light chain of human ferritin via a linker;
(2) scFab of an antibody that is capable of binding (e.g., specifically binding) to an immune cell fused to the light chain of human ferritin via a linker;
(3) scFab of an antibody that is capable of binding (e.g., specifically binding) to a tumor-associated antigen fused to the N-half of a light chain of human ferritin via a linker; and
(4) scFc fused to the C-half of light chain of human ferritin via a linker,
wherein the ratio of polypeptides of (3) and (4) is 1:1.

### Set 12

(1) scFab of an antibody that is capable of binding (e.g., specifically binding) to a tumor-associated antigen fused to the light chain of human ferritin via a linker;
(2) scFab of an antibody that is capable of binding (e.g., specifically binding) to an immune cell fused to the light chain of human ferritin via a linker;
(3) scFab of an antibody that is capable of binding (e.g., specifically binding) to an immune cell fused to the light chain of the N-half of human ferritin via a linker; and
(4) scFc fused to the C-half of light chain of human ferritin via a linker,
wherein the ratio of polypeptides of (3) and (4) is 1:1.

### Set 13

(1) scFab of an antibody that is capable of binding (e.g., specifically binding) to a tumor-associated antigen fused to the light chain of human ferritin via a linker; and
(2) scFab of an antibody that is capable of binding (e.g., specifically binding) to a tumor-associated antigen fused to one terminus of the light chain of human ferritin via a linker, and an scFc fused to the other terminus of the light chain of the human ferritin via a linker.

### Set 14

(1) scFab of an antibody that is capable of binding (e.g., specifically binding) to an immune cell fused to the light chain of human ferritin via a linker; and
(2) scFab of an antibody that is capable of binding (e.g., specifically binding) to an immune cell fused to one terminus of the light chain of human ferritin via a linker, and an scFc fused to the other terminus of the light chain of the human ferritin via a linker.

### Set 15

(1) scFab of an antibody that is capable of binding (e.g., specifically binding) to a tumor-associated antigen fused to the light chain of human ferritin via a linker; and
(2) scFab of an antibody that is capable of binding (e.g., specifically binding) to an immune cell fused to one terminus of the light chain of human ferritin via a linker, and an scFc fused to the other terminus of the light chain of the human ferritin via a linker.

### Set 16

(1) scFab of an antibody that is capable of binding (e.g., specifically binding) to an immune cell fused to the light chain of human ferritin via a linker; and
(2) scFab of an antibody that is capable of binding (e.g., specifically binding) to a tumor-associated antigen fused to one terminus of the light chain of human ferritin via a linker, and an scFc fused to the other terminus of the light chain of the human ferritin via a linker.

Upon expression, the encoded polypeptides self-assemble to form immune cell-engaging or activating MBs.

### Example 2. Target Binding of MBs Determined by Biolayer Interferometry

The binding kinetics and affinity of exemplary immune cell-engaging MBs generated as described in Example 1 to antigens on immune cells and/or to tumor-associated antigens can be determined by biolayer interferometry (BLI). For example, the binding characteristics of IgG antibodies (from which the scFabs within the immune cell-engaging MBs are derived) can also be determined for comparison.

For example, Ni-NTA biosensors can be coated with His-tagged antigens (e.g., extracellular domains of antigens on immune cells or extracellular domains of tumor-associated antigens). Coated biosensors can be dipped into wells containing serial dilutions of the MBs (or of the antibody controls) in a buffer such as a phosphate-buffered saline (PBS) buffer comprising Tween 20 and optionally comprising bovine serum albumin (BSA) during an association phase and during a dissociation phase.

Biosensors can be regenerated between experiments, for example, by applying a glycine solution several times and by recharging in NiSO₄.

Target binding can be evaluated based on the maximal association binding response at the end of the association phase, the dissociation rate (k_{off}), and/or the equilibrium dissociation constant (K_{D}) calculated using a 1:1 fitting model. A maximal association binding response of less than 0.1 nm for a concentration of 20 nM Multabody can be classified as “non-binding.”

### Example 3. Binding of MBs to Fc Receptors Determined by Biolayer Interferometry

The binding kinetics and affinities of various MBs generated as described in Example 1 to various Fc receptors can be determined by biolayer interferometry.

MBs tested in this Example contain polypeptides comprising one or more Fc chains. As controls for comparison, MBs without Fc-containing polypeptides and/or IgG antibodies of the same class as the Fc chains used in the MBs can be used.

Binding to various Fc receptors can be determined, for example, to human Fc receptors, such as: human Fc gamma receptor type I (hFcγRI), hFcγRIIa, hFcγRIIb, hFcγRIIIa, hFcγRIIIb, and human neonatal Fc receptor (hFcRn). To assess the possibility and relevance of testing the MBs in animal models, binding to non-human Fc receptors can also be assessed, such as to cynomolgus Fc receptors (e.g., cynomolgus FcγRI (cFcγRI), cFcγRIIa, cFcγRIIb, cFcγRIII, and cFcRn) and/or to mouse Fc receptors (e.g., mouse FcγRI (mFcγRI), mFcγRIIb, mFcγRIII, mFcγRIV, and mFcRn).

Experiments can be performed similarly as described in Example 2, except that His-tagged Fc receptors are coated onto Ni-NTA biosensors and titrated with test MBs at various concentrations. To assess the potential of the MBs to undergo endosomal recycling, binding to FcRn can be measured at pH 6.0 for association and at pH 7.4 for dissociation.

### Example 4. Assessment of In Vitro Tumor Cell Cytotoxicity Facilitated by Natural Killer (NK)-Cell Engaging MBs

The ability of natural killer (NK)-cell engaging MBs to facilitate killing of B-cell lymphoma cells expressing CD20 and CD37 can be assessed. MBs are generated as described in Example 1, using an scFab of an antibody against NKG2D (which is expressed on natural killer cells and some subsets of T cells) and an scFab of an α-CD20 or an α-CD37.

Cytotoxicity is evaluated using co-culture assays with (1) human natural killer cells (e.g., primary NK cells, available at StemCell Technologies) co-cultured with (2) B-cell lymphoma target cells. For the B-cell lymphoma cells, CD20+, CD37+ Raji and/or Daudi B-cell lymphoma cell lines (commercially available, e.g., at ATCC (Manassas, VA)) may be used.

B-cell lymphoma target cells are co-cultured with human natural killer cells (e.g., at an effector cell to target cell ratio of about 1:1) and treated with serial dilutions of MBs or a control molecule (e.g., control Multabody (α-respiratory syncytial virus (RSV)). After incubation at about 37° C. for at least about 3 hours, cell viability of B-cell lymphoma target cells is measured (e.g., using a kit such as Invitrogen Calcein-AM™). Calcein-AM is a cell-permeant dye used as a marker indicative of cell viability.

Increased calcein release in samples incubated with NK-cell engaging MBs (as compared to that in samples incubated with control molecules) would indicate that NK-cell engaging MBs are more efficient at killing B-cell lymphoma target cells.

### Example 5. Assessment of In Vitro Antibody-Dependent Cellular Cytotoxicity Facilitated by Natural Killer (NK)-Cell Engaging MBs

To assess the ability of wild type Fc molecules present on MBs to induce antibody-dependent cellular cytotoxicity (ADCC), tumor-binding MBs comprising wild type IgG1 Fc's were generated and assessed in an in vitro ADCC reporter assay.

Multabody generation: MBs containing CD37 Fabs and wild type IgG1 Fcs were generated similarly as described in Example 1, with the following formats:

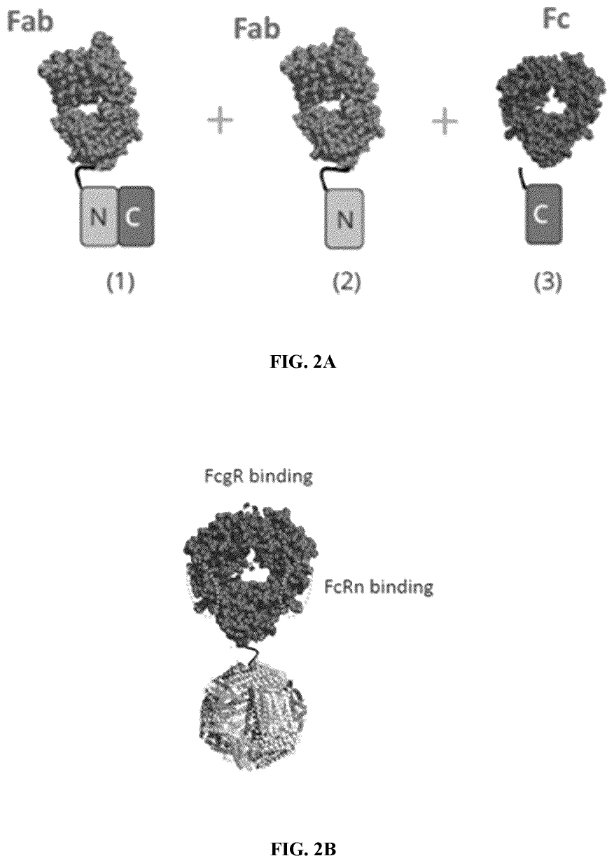

Format 1: MBs comprising the following fusion proteins were constructed. (See FIG. 2A)
(1) scFab of a CD37 antibody fused to the N-terminus of a human ferritin light chain via a (GGGS)ₙ linker;
(2) scFab of a CD37 antibody fused to the N-terminus of the N-terminal half of a human ferritin light chain via a (GGGS)ₙ linker; and
(3) scFc (IgG1) fused to the N-terminus of the C-terminal half of a human ferritin light chain via a (GGGS)ₙ linker,
wherein the ratio of polypeptides (2) and (3) is 1:1.

In the resulting MBs with Format 1, the Fcs are oriented such that the FcγR binding pocket is away from the ferritin core of the MB (see FIG. 2B).

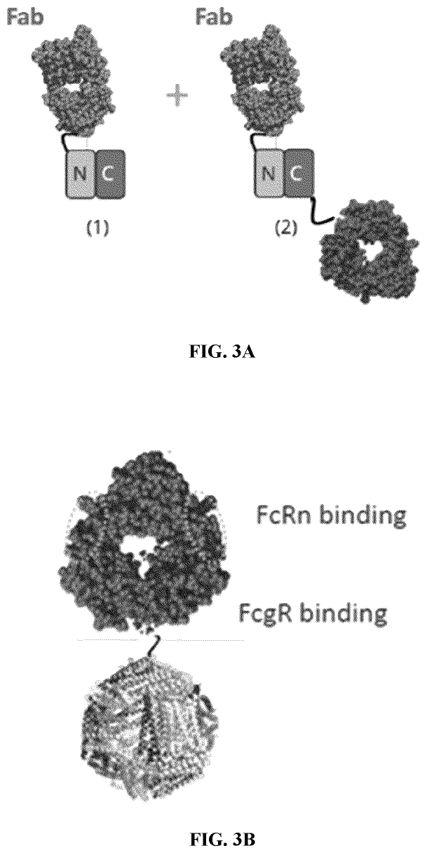

Format 2: MBs comprising the following fusion proteins were constructed. (See FIG. 3A)
(1) scFab of a CD37 antibody fused to the N-terminus of a light chain of human ferritin via a (GGGS)ₙ linker; and
(2) a light chain of human ferritin fused to:
   scFab of a CD37 antibody (fused to the N-terminus of the human ferritin light chain via a (GGGS)ₙ linker)
   scFc (IgG1) (fused to the C-terminus of the human ferritin light chain via a (GGGS)ₙ linker).

In the resulting MBs with Format 2, the Fcs are oriented such that the FcγR binding pocket is facing toward the ferritin core of the MB (see FIG. 3B).

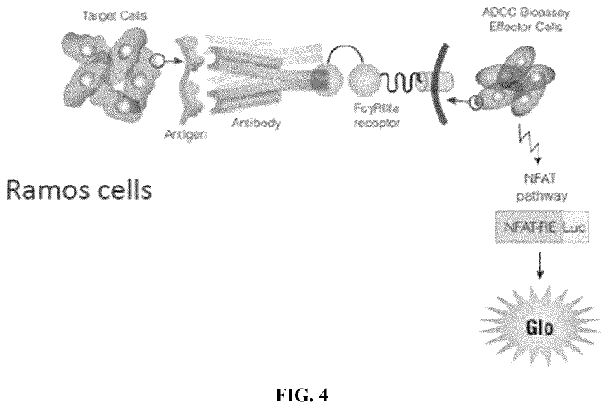

ADCC function: The ability of the α-CD37/Fc MBs to induce antibody-dependent cellular cytotoxicity (ADCC) was then tested in an in vitro assay using Ramos target cells and ADCC Bioassay effector cells. Successful induction of ADCC via the Fc of a test molecule (such as a Multabody as disclosed herein) leads to NFAT activation in the effector cells, which in this assay system results in luciferase expression driven by an NFAT-responsive element. (See FIG. 4.) Thus, luciferase signal in this assay is indicative of ADCC function.

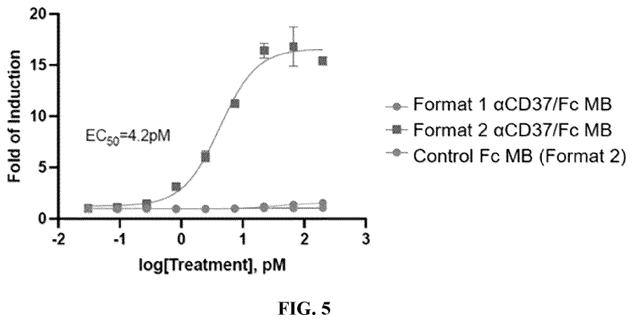

Format 2 α-CD37/Fc MBs demonstrated an ability to induce ADCC, with an EC₅₀ of about 4.2 pM. By contrast, Format 1 α-CD37/Fc MBs did not induce ADCC in this assay. (See FIG. 5.)

Thus, the present Example demonstrates that certain MBs of the present disclosure can induce ADCC in vitro.

### Example 6. Assessment of In Vitro Tumor Cell Cytotoxicity Facilitated by T Cell-Engaging MBs

The ability of T cell-engaging MBs to facilitate killing of B-cell lymphoma cells expressing CD20 and CD37 can be assessed. MBs are generated as described in Example 1, using an scFab of an antibody against CD3 (which is expressed on T cells) and an scFab of an α-CD20 or an α-CD37.

Cytotoxicity is evaluated using co-culture assays with (1) human peripheral blood mononuclear cells (PBMCs) or purified human T cells co-cultured with (2) B-cell lymphoma target cells. PBMCs are obtained from healthy donor blood using, e.g., the Ficoll-Paque Plus kit (GE Healthcare), and CD8+ T cells are purified using a T cell isolation kit (e.g., from StemCell Technologies). For the B-cell lymphoma cells, CD20+, CD37+ Raji and/or Daudi B-cell lymphoma cell lines (commercially available, e.g., at ATCC (Manassas, VA)) may be used.

B-cell lymphoma target cells are co-cultured with (1) PBMCs (e.g., at an effector cell to target cell ratio of about 25-30:1) or (2) IL-2 activated PBMCs (e.g., at an effector cell to target cell ratio of about 15:1) and treated with serial dilutions of MBs or a control molecule (e.g., a bispecific α-CD3/α-CD20 and/or bispecific α-CD3/α-CD37). After incubation at about 37° C. overnight, lactate dehydrogenase (LDH) release from B-cell lymphoma target cells is measured (e.g., using a kit such as Promega's LDH-Glo™). LDH release is used as a marker indicative of target cell killing.

Increased LDH release in samples incubated with T cell-engaging MBs (as compared to that in samples incubated with control molecules) would indicate that T-cell engaging MBs are more efficient at killing B-cell lymphoma target cells.

For experiments with purified T cells, B-cell lymphoma target cells are pre-labeled with carboxyfluorescein succinimidyl ester (CFSE) dye or CellTrace fluorescent stains (ThermoFisher Scientific) to distinguishing between B-cell lymphoma target cells and T cells. Labeled B-cell lymphoma target cells are co-cultured with purified T cells and are incubated at approximately 37° C. for at least about two hours. Cell-killing is assessed by staining the cells for propidium iodide (PI), which stains dead cells, and then assessing staining by flow cytometry.

Increased PI staining in samples incubated with T cell-engaging MBs (as compared to that in samples incubated with control molecules) would indicate that T cell-engaging MBs are more efficient at killing B-cell lymphoma target cells.

### Example 7. Pharmacokinetics of MBs in a Non-Human Animal Models

The pharmacokinetics (PK) of exemplary immune cell engaging MBs generated as described in Example 1 can be analyzed in one or more non-human animal models, such as mice and/or non-human primates (e.g., cynomolgus monkeys).

Animals receive a single bolus injection of the Multabody or a control molecule (e.g., an IgG) by systemic rout of administration (e.g., intravenous). Blood samples are collected at various timepoints, e.g., at around of 3 h, 24 h, 48 h, 72 h, 5 days, 7 days, 15 days, and 21 days after injection.

Multi-dose studies can also be performed, with the first and second doses spaced apart, e.g., by a number of days or weeks.

Plasma samples may be obtained from blood samples and stored frozen until use.

To measure levels of MBs or control molecules in plasma by enzyme-linked immunosorbent assay (ELISA), recombinant tumor associated antigens (e.g., CD20 or CD37) are coated onto plates and left overnight at 4° C. After washing twice (e.g., with a solution such as phosphate-buffered saline (PBS)-Tween-20, the plate is blocked with a bovine serum albumin (BSA) solution in PBS for 1 hour at room temperature and subsequently washed twice. Plasma samples are diluted in PBS/Tween-20/BSA solution, added to wells, and incubated for 1 h at room temperature on a shaker. After another wash step, bound molecules are detected by incubation with diluted goat polyclonal α-human Fc-HRP secondary antibody. After a further wash step, a substrate used for detection is added, and the absorbances at 450 nm were read using a microplate reader. A calibration curve is prepared using dilutions of MBs in PBS/Tween-20/BSA solution.

Half-lives of MBs in animals can be calculated based on these measurements, and the half-lives of MBs can be compared that of IgG molecules.

### Example 8. Therapeutic Effect of Immune Cell-Engaging MBs in a Xenograft Mouse Model

The therapeutic effect of exemplary MBs can be evaluated in a B-cell lymphoma Xenograft model. Daudi and Raji human Burkitt lymphoma cell lines (commercially available from ATCC) are grown as suspension cultures. For the solid tumor model, approximately 5×10⁶ Raji cells or x Daudi cells are injected sub-cutaneously into the flank of SCID mice. Tumor volume is measured twice weekly using calipers (solid tumor model). For the disseminated tumor model, approximately 1.5×10⁷ Daudi cells or approximately 2.5×10⁶ Raji cells are injected intravenously via the tail vein into SCID mice. Mice are examined daily for hind-leg paralysis and are weighed weekly. MBs generated as described in Example 1 (or control MBs (such MBs that do not contain a tumor-binding moiety) and/or IgG1 control antibodies that bind to tumors)) are injected at several time points (e.g., 1 day, 5 days, 10 days, and 15 days) after inoculation with tumor cells. Several dose amounts of MBs can be tested. Animals are killed when they have lost 20% of their initial weight, tumors have reached maximal tumor volume or they have developed hind-leg paralysis.

Tumor growth, survival curves and weight loss are compared across treatment groups. Reduction in tumor growth and/or improved survival in animals treated with MBs (as compared with those treated with control molecules) would suggest improved efficacy of immune cell-engaging MBs.

### Example 9. Tumor Cell Apoptosis Induced by q-CD37 MBs by CD37 Receptor Clustering

The ability of αCD37/Fc MBs (Format 2) (generated as described in Example 5) to induce apoptosis via CD37 receptor clustering in CD37+ tumor cells was assessed. α-CD37/Fc MBs were generated as described in Example 5.

The apoptotic activity of α-CD37 IgGs and α-CD37/Fc MBs were determined using an apoptosis assay.

Apoptosis assay. Daudi or Ramos cells were used as the CD37+ target tumor cells, and Annexin-V staining was used to identify apoptotic cells. Target cells were incubated with serial dilutions of test IgGs or MBs in 96-well U-bottom plates for 24 hours at 37° C., 5% CO₂. At the end of the incubation period, flow cytometry was performed to assess the viability of the target cells. Cells were washed with chilled phosphate-buffered saline (PBS) and stained with LIVE/DEAD (L/D) Violet Viability Dye for 30 minutes on ice. After L/D staining, the cells were washed and then stained with FITC-conjugated Annexin-V dye for 15 minutes on ice. The FITC signal was measured on a CytoFLEX flow cytometer (Beckman Coulter). FlowJo Software was used for data analysis. The FITC-Annexin-V+ gate was determined based on the Florescence Minus One (FMO) and no treatment control, and % FITC-Annexin-V+ cells were calculated according to the % of single cells present within the FITC-Annexin-V+ gate.

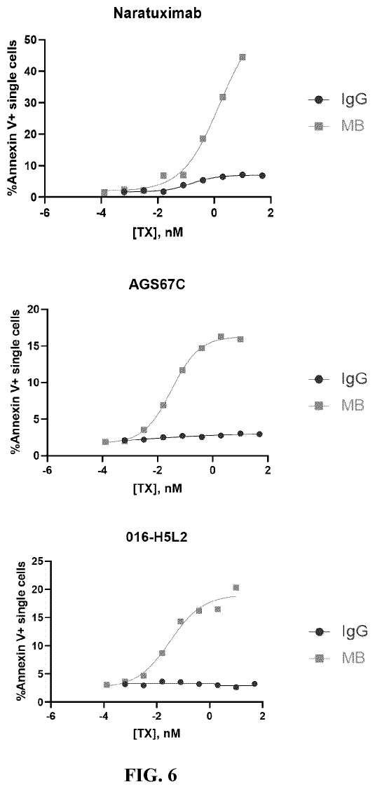

Results. Increased fluorescence was observed in target cell samples incubated with αCD37/Fc MBs as compared to that observed in target cell samples incubated with parent IgG. (FIG. 6). These results demonstrate that all tested αCD37/Fc MBs induced potent tumor cell apoptosis and showed superior tumor cell killing as compared to parent IgGs. Without wishing to be bound by any particular theory, this increase in apoptotic ability may be due to enhanced receptor clustering achieved by the multi-valency of MBs.

αCD37/Fc MBs demonstrated an ability to induce apoptosis with an EC₅₀ of about 32 pM. (See Table 8).

TABLE 8
Half maximal effective concentration (EC₅₀) of
apoptosis by CD37 IgG vs MBs on Daudi cells

| Molecule | | EC₅₀ (nM) |
|---|---|---|
| IgG | Naratuximab | — |
| | AGS67C | — |
| | 016-H5L2 | — |
| αCD37/ | Naratuximab | — |
| Fc MBs | AGS67C | 0.032 |
| | 016-H5L2 | 0.032 |

### Example 10. In Vitro Antibody-Dependent Cellular Cytotoxicity Facilitated by Natural Killer (NK)-Cell Engaging MBs

αCD37/Fc MBs (Format 2) generated as described in Example 5 were assessed for ability to induce antibody-dependent cellular cytotoxicity (ADCC) in an in vitro cytotoxicity assay. Parental IgGs and control MBs (using α-RSV Fabs) were used as controls.

ADCC assay. Ramos cells were used as the target cells, and primary human NK cells (STEMCELL Technologies) were used as the effector cells. On the day before the experiment, frozen primary human NK cells were thawed and rested in RPMI-1640 media with 20% fetal bovine serum (FBS) and 5 U/ml human IL-2 overnight at 37° C., 5% CO₂. On the day of the experiment, target cells were labeled with 10 mM Calcein-AM dye for 30 minutes at 37° C., 5% CO₂. The labeled target cells were then washed twice with RPMI-1640 media. Target cells and effector cells were adjusted to the appropriate cell concentrations. Assay plates were assembled by combining target cells, effector cells, and serial dilutions of test IgGs or MBs into 96-well U-bottom plates so that the effector cell to target cell ratio was 1:1 (10000 cells each per well). The assay plates were incubated at 37° C., 5% CO₂ for 4 hours. Fluorescence signal from the released Calcein-AM dye was then measured by a microplate reader at Ex488/Em530. Increased calcein release, measured as increased fluorescence signal in samples, indicates more efficient at killing target cells.

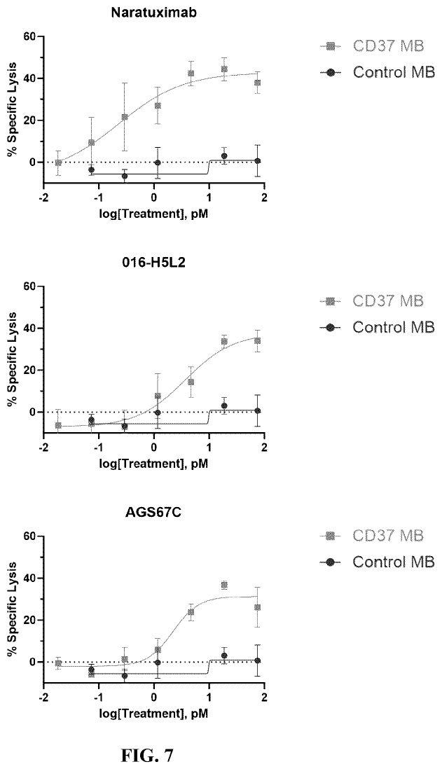

Results. Increased fluorescence (indicative of more efficient killing) was observed in target cell samples incubated with αCD37/Fc MBs as compared to that in target cell samples incubated with control MBs (FIG. 7. These results demonstrate that all tested αCD37/Fc MBs induced potent tumor cell ADCC.

No increase in fluorescence was observed in target cell samples incubated with control α-respiratory syncytial virus (RSV) MBs comprising an active wild-type Fc. (See FIG. 7). These results demonstrate that in the absence of target, MBs do not induce non-specific activation of ADCC.

Table 9 shows the calculated EC₅₀ values for ADCC activity induced by αCD37/Fc MBs or by parental IgGs.

TABLE 9
Half maximal effective concentration
(EC₅₀) of ADCC by CD37 IgG vs MBs on Ramos cells

| Molecule | | EC₅₀ (pM) |
|---|---|---|
| IgG | Naratuximab | 5.97 |
| | AGS67C | 9.33 |
| | 016-H5L2 | 8.34 |
| αCD37/ | Naratuximab | 0.21 |
| Fc MBs | AGS67C | 2.27 |
| | 016-H5L2 | 3.68 |

### Example 11. Assessment of In Vitro C1q Binding and Complement-Dependent Cytotoxicity Facilitated by Human Complement Engaging MBs

### Assessment of C1q Binding by ELISA.

αCD37/Fc MBs (Format 2) generated as described in Example 5 were assessed for ability to bind C1q, as determined by ELISA. Parental IgGs were used as controls.

Serial dilutions of test IgGs and MBs were coated on 96-well plates at 4° C. overnight. The next day, the assay plates were blocked with 3% bovine serum albumin (BSA) for 1 hour at room temperature. After washing out the blocking solution, 2 μg/ml of recombinant human C1q (Complement Technology Inc.) was added to the assay plate and incubated for 1 hour at room temperature to allow binding to coated test subjects. HRP-conjugated sheep-α-human C1q secondary antibody was then used to detect C1q binding through TMB/HRP reactions.

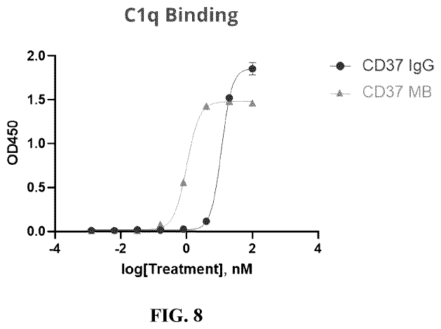

C1q protein was determined to bind to α-CD37/Fc MBs and parent IgGs (see FIG. 8).

### Assessment of Complement-Dependent Cytotoxicity (CDC) Activity by CDC Assay.

αCD37/Fc MBs were also assessed for the ability to induce complement-dependent cytotoxicity (CDC) in an in vitro CDC assay. Parental IgGs and control MBs (using α-respiratory syncytial virus (RSV) Fabs) were used as controls.

Daudi cells were used as the target cells, and normal human serum (Complement Technology) was the source of human complement proteins. Target cells were incubated with serial dilutions of test IgGs/MBs and normal human serum (final concentration of 20%) in 96-well U-bottom plates for 2 hours at 37° C., 5% CO₂. After the incubation period, flow cytometry was performed to measure the viability of the target cells. Cells were washed and stained with propidium iodide (PI) viability dye for 10 minutes on ice. The PI fluorescent signal was measured on CytoFLEX flow cytometer (Beckman Coulter). FlowJo Software was used for data analysis. The PI+ gate was determined based on the FMO and no treatment control and % PI+ cells were calculated according to the % of single cells present within the PI+ gate. PI is a fluorescent DNA-binding dye that freely penetrates cell membranes of dead or dying cells, but is excluded from viable cells. Thus, increased PI fluorescent signal in samples indicate more efficient killing of target cells.

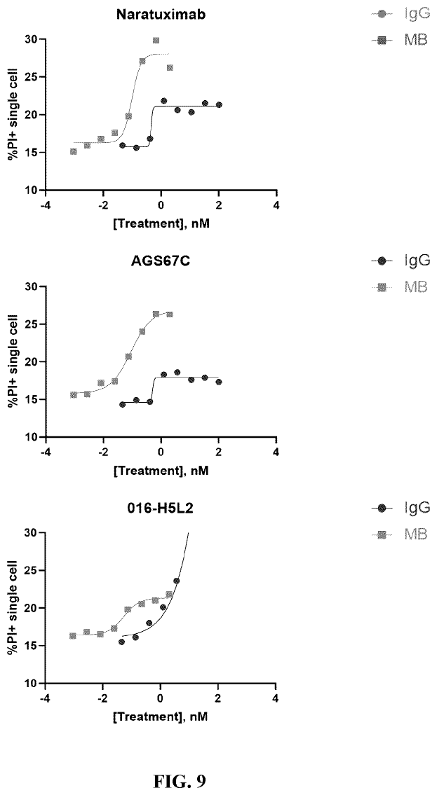

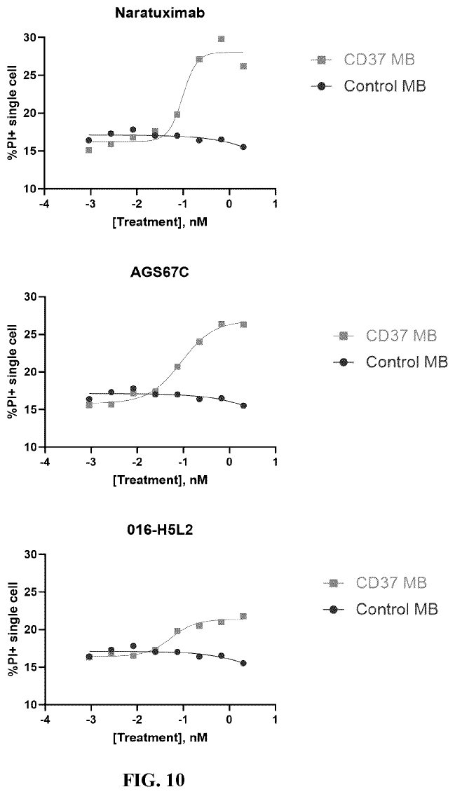

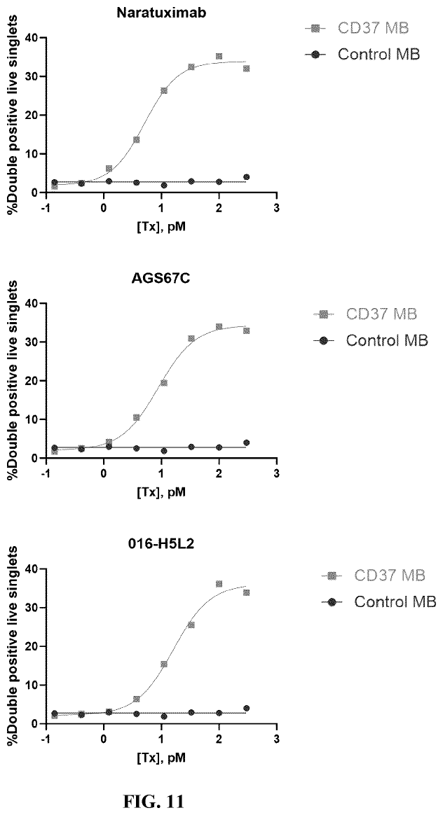

Results. Increased PI fluorescent signal (indicative of more killing) was observed in target cell samples incubated with αCD37/Fc MBs as compared to that observed in target cell samples incubated with parental IgGs (FIG. 9) or as compared to control MBs (FIG. 10). These results demonstrate that all tested α-CD37/Fc MBs induced more potent CDC activity as compared to parental IgGs or to control MBs.

No increase in PI fluorescent signal was observed in target cell samples incubated with control MBs comprising an active wild-type Fc. (FIG. 10). These results demonstrate that in the absence of target, MBs do not induce non-specific activation of CDC.

Table 10 shows the calculated EC50 values for CDC activity induced by α-CD37/Fc MBs or by parental IgGs.

TABLE 10
Half maximal effective concentration
(EC₅₀) of CDC by CD37 IgG vs MBs on Daudi cells

| Molecule | | EC₅₀ (nM) |
|---|---|---|
| IgG | Naratuximab | 0.45 |
| | AGS67C | 0.51 |
| | 016-H5L2 | — |
| αCD37/ | Naratuximab | 0.10 |
| Fc MBs | AGS67C | 0.09 |
| | 016-H5L2 | 0.05 |

### Example 12. Assessment of In Vitro Antibody-Dependent Cellular Phagocytosis Facilitated By Macrophage Engaging MBs

αCD37/Fc MBs (Format 2) generated as described in Example 5 were assessed for ability to induce antibody-dependent cellular phagocytosis (ADCP) in an in vitro ADCP assay. Parental IgGs or control MBs (using α-RSV Fabs) were used as controls.

ADCP assay. The ADCP activity of IgGs and MBs were determined by the ADCP Assay using Ramos cells as target cells and THP-1 cells as effector cells. Target cells were labeled with pHrodo Green AM Intracellular pH Indicator Dye (Invitrogen) and effector cells were labeled with CellTrace Violet Dye. After washing the labeled cells, the assay plates were assembled by combining labeled target cells, labeled effector cells with serial dilutions of test IgGs/MBs into 96-well U-bottom plates. The samples were incubated for 1 hour at 37° C., 5% CO₂. After incubation, the samples were stained with L/D Far Red viability dye for 15 minutes on ice before analysis on a CytoFLEX flow cytometer (Beckman Coulter). FlowJo Software was used for data analysis and pHrodo Green AM and CellTrace Violet+/− gates were determined according to FMO and no treatment controls. ADCP activity was evaluated based on the percentage of pHrodo Green AM/CellTrace Violet double positive cells among all live single cells.

Results. A higher percentage of pHrodo Green AM/CellTrace Violet double positive cells (among all live single cells) was observed in target cell samples incubated with αCD37/Fc MBs as compared to that observed in target cell samples incubated with control MBs (FIG. 11). These results demonstrate that all tested αCD37/Fc MBs induced potent tumor cell ADCP as compared to control α-respiratory syncytial virus (RSV) MBs.

No increase in percentage of pHrodo Green AM/CellTrace Violet double positive cells in all live single cells was observed in target cell samples incubated with control MBs comprising an active wild-type Fc (FIG. 11). These results demonstrate that in the absence of target, MBs do not induce non-specific activation of ADCP.

Table 11 shows the calculated EC50 values for ADCP activity induced by αCD37/Fc MBs or by parental IgGs.

TABLE 11
Half maximal effective concentration
(EC₅₀) of ADCP by CD37 IgG vs MBs on Ramos cells

| Molecule | | EC₅₀ (pM) |
|---|---|---|
| IgG | Naratuximab | 37.44 |
| | AGS67C | 19.08 |
| | 016-H5L2 | 21.36 |
| αCD37/ | Naratuximab | 5.12 |
| Fc MBs | AGS67C | 8.83 |
| | 016-H5L2 | 16.08 |

### Example 13. Assessment of Bispecific Format MBs

### Generation of MBs.

This Example describes the generation of bispecific MBs capable of targeting both the CD37 tumor antigen and the NKp46 NK cell activating receptor. The binding properties, ADCC activity, and apoptotic activity of these bispecific MBs was also assessed in comparison to control CD37 monospecific MBs.

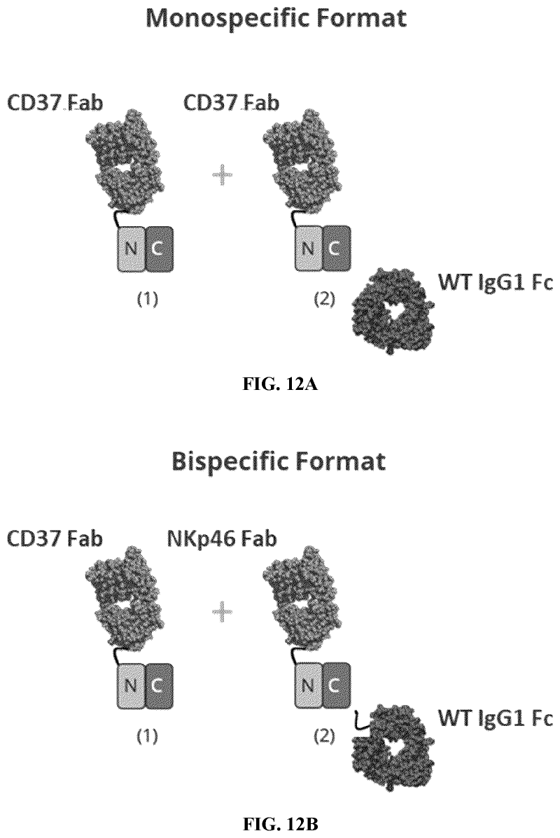

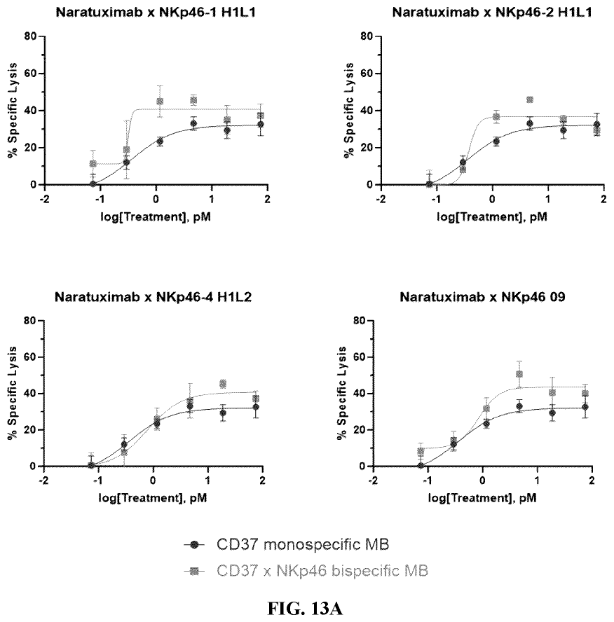

Monospecific Format: αCD37/Fc MBs (Format 2) were generated as described in Example 5. (See FIG. 12A).

Bispecific Format: Bispecific MBs containing CD37 Fabs, NKp46 Fabs, and wild type IgG1 Fcs were generated similarly as described in Example 1, with the following format. (See FIG. 12B).
(1) scFab of a CD37 antibody fused to the N-terminus of a human ferritin light chain via a (GGGS)ₙ linker; and
(2) a light chain of human ferritin fused to:
   scFab of a NKp46 antibody (fused to the N-terminus of the human ferritin light chain via a (GGGS)ₙ linker)
   scFc (wt IgG1) (fused to the C-terminus of the human ferritin light chain via a (GGGS)ₙ linker).

A-CD37 Fabs and α-NKp46 Fabs listed in Table 12 were used for the bispecific CD37/Nkp46 MBs.

TABLE 12
A-CD37 and A-NKp46 Fabs used in Bispecific Format MBs

| A-CD37 Fabs | A-NKp46 Fabs |
|---|---|
| Naratuximab | NKp46-1 H1L1 |
| AGS67C | NKp46-2 H1L1 |
| 016-H5L2 | NKp46-4 H1L2 |
| — | NKp46 09 |

### Assessment of In Vitro Binding of CD37/NKp46 MBs to Recombinant Human NKp46 by Biolayer Interferometry

The binding kinetics and affinity of MBs to recombinant human NKp46 (hNKp46) were determined by biolayer interferometry (BLI) using an Octet RED96 instrument. Ni-NTA biosensors were coated with hNKp46-His (extracellular domain of hNKp46 with a C-terminal polyhistidine tag) to reach a signal response of 0.8 nm. The coated biosensors were dipped into wells containing serial dilutions of the test MBs (20-10-5-2.5-1.25-0.63 nM) in PBS-0.02% T-0.01% BSA (PBS supplemented with 0.02% (v/v) Tween 20 and 0.01% (w/v) BSA) for 180 seconds (association phase) and then into PBS-0.02% T-0.01% BSA for 180 seconds (dissociation phase). All measurements were performed at 30° C. in PBS-0.02% T-0.01% BSA, pH 7.4, with shaking speed 1000 rpm and monitored in real time. Biosensors were regenerated between experiments by applying 10 mM glycine, pH 1.7, for 5 seconds for four times, followed by recharging with 10 mM NiSO₄ for 1 minute. Target binding was evaluated based on the maximal association binding response at the end of the association phase, the dissociation rate (k_{off}), and/or the equilibrium dissociation constant (K_{D}) calculated using a 1:1 fitting model. A maximal association binding response of less than 0.1 nm when test Multabody at 20 nM was classified as “non-binding.”

All CD37/NKp46 bispecific MBs exhibited binding in the picomolar or sub-picomolar range. (See Table 13).

TABLE 13
K_{D}, kₒₙ, and k_{dis} values for CD37/NKp46 bispecific MBs

| | K_{D} | Kₒₙ | K_{dis} |
|---|---|---|---|
| MB | (M) | (1/Ms) | (1/s) |
| Naratuximab × | <1.0E−12 | 3.650E06 | <1.0E−07 |
| NKp46-1 H1L1 | | | |
| Naratuximab × | <1.0E−12 | 2.198E06 | <1.0E−07 |
| NKp46-2 H1L1 | | | |
| Naratuximab × | 1.747E−11 | 1.957E06 | 3.419E−05 |
| NKp46-4 H1L2 | | | |
| Naratuximab × | <1.0E−12 | 1.633E06 | <1.0E−07 |
| NKp46 09 | | | |
| AGS67C × NKp46-1 H1L1 | <1.0E−12 | 1.744E06 | <1.0E−07 |
| AGS67C × NKp46-2 H1L1 | <1.0E−12 | 1.237E06 | <1.0E−07 |
| AGS67C × NKp46-4 H1L2 | <1.0E−12 | 1.240E06 | <1.0E−07 |
| AGS67C × NKp46 09 | <1.0E−12 | 1.085E06 | <1.0E−07 |
| 016-H5L2 × NKp46-1 H1L1 | <1.0E−12 | 2.147E06 | <1.0E−07 |
| 016-H5L2 × NKp46-2 H1L1 | <1.0E−12 | 1.253E06 | <1.0E−07 |
| 016-H5L2 × NKp46-4 H1L2 | <1.0E−12 | 1.670E06 | <1.0E−07 |
| 016-H5L2 × NKp46 09 | <1.0E−12 | 1.057E06 | <1.0E−07 |

### Assessment of In Vitro Antibody-Dependent Cellular Cytotoxicity Facilitated by Natural Killer (NK)-Cell Engaging Bispecific MBS.

CD37/NKp46 bispecific MBs comprising wild type IgG1 Fc's were assessed for ability to induce antibody-dependent cellular cytotoxicity (ADCC) in an in vitro ADCC assay similarly as described in Example 10, with Ramos cells used as the target cells and primary human NK cells (STEMCELL Technologies) used as the effector cells.

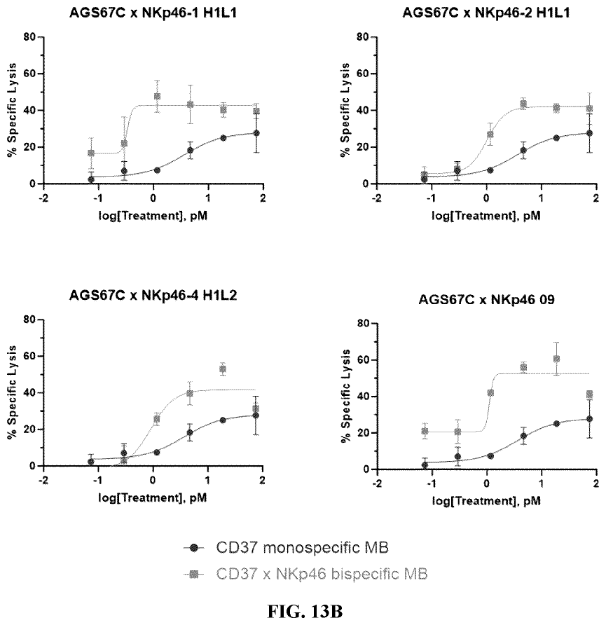

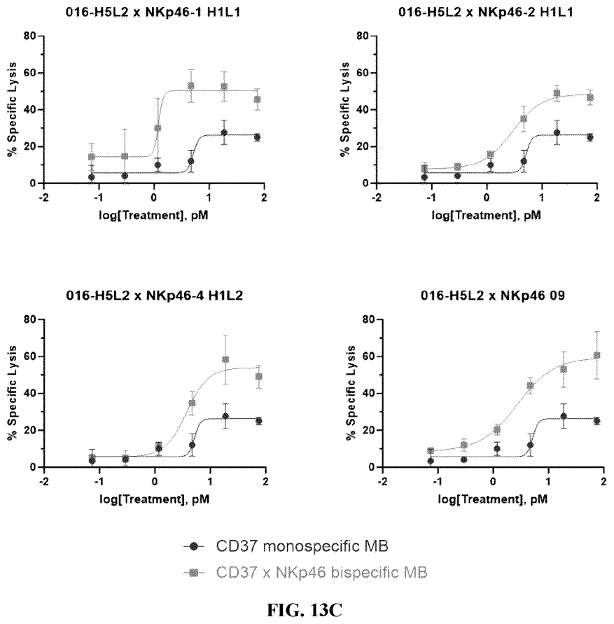

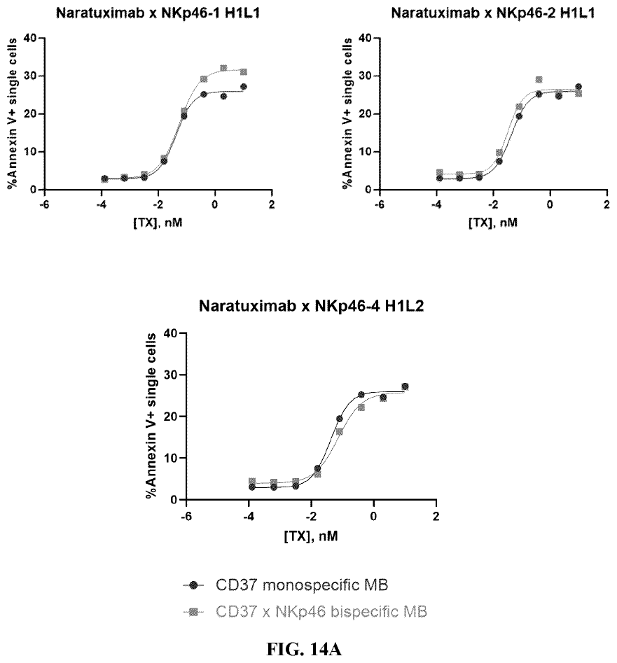

Increased fluorescence (indicative of more efficient killing of target cells) was observed in target cell samples incubated with CD37/NKp46 bispecific MBs as compared to that observed in target cell samples incubated with monospecific CD37 MBs (FIGS. 13A-13C). These results demonstrate that all CD37/NKp46 bispecific MBs tested induced potent tumor cell ADCC and enhanced NK-mediated killing of tumor cells relative to that induced by monospecific CD37 MBs.

Table 14 shows the calculated EC50 values for ADCC activity induced by monospecific CD37 MBs or CD37/NKp46 bispecific MBs.

TABLE 14
Half maximal effective concentration (EC₅₀) of ADCC
by monospecific vs bispecific MBs on Ramos cells

| Molecule | | EC₅₀ (pM) |
|---|---|---|
| CD37 monospecific | Naratuximab | 0.363 |
| MBs | AGS67C | 3.626 |
| | 016-H5L2 | 5.167 |
| CD37/NKp46 | Naratuximab × NKp46-1 H1L1 | 0.316 |
| bispecific MBs | Naratuximab × NKp46-2 H1L1 | 0.374 |
| | Naratuximab × NKp46-4 H1L2 | 0.777 |
| | Naratuximab × NKp46 09 | 0.843 |
| | AGS67C × NKp46-1 H1L1 | 0.335 |
| | AGS67C × NKp46-2 H1L1 | 1.014 |
| | AGS67C × NKp46-4 H1L2 | 0.904 |
| | AGS67C × NKp46 09 | 1.109 |
| | 016-H5L2 × NKp46-1 H1L1 | 1.204 |
| | 016-H5L2 × NKp46-2 H1L1 | 2.910 |
| | 016-H5L2 × NKp46-4 H1L2 | 3.731 |
| | 016-H5L2 × NKp46 09 | 2.718 |

### Assessment of In Vitro Tumor Cell Apoptosis Induced by CD37 Receptor Clustering by NK-Cell Engaging Bispecific MBS

The ability of CD37/NKp46 bispecific MBs to induce apoptosis in CD37+ tumor cells by CD37 receptor clustering was assessed using an assay similarly as described in Example 9, with Daudi cells as the target cells and Annexin-V staining used to identify apoptotic cells.

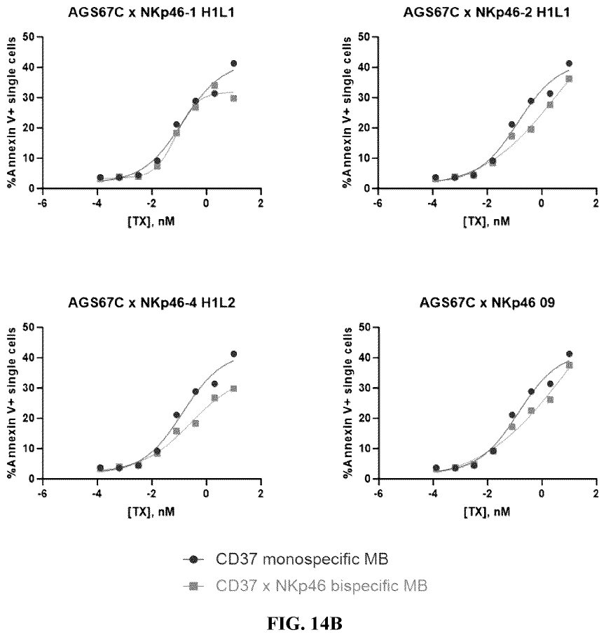

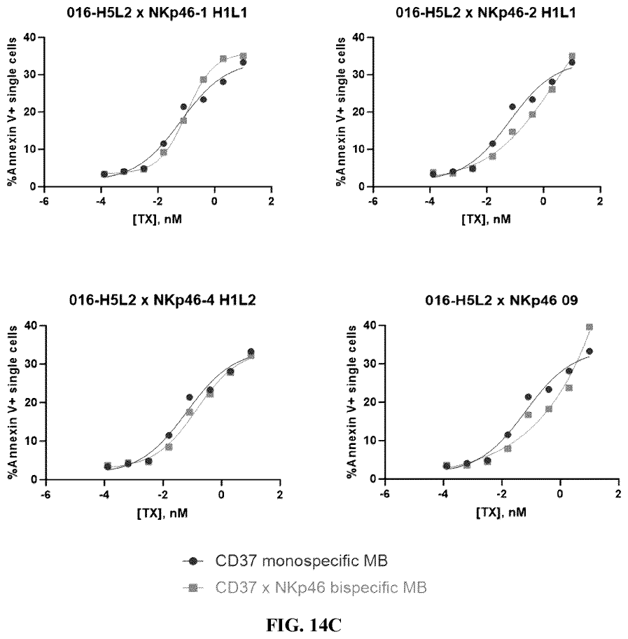

CD37/NKp46 bispecific MBs showed comparable apoptotic activity to CD37 monospecific MBs (see FIGS. 14A-14C).

Table 15 shows the calculated EC50 values for apoptotic activity induced by CD37 monospecific MBs or CD37/NKp47 bispecific MBs.

TABLE 15
Half maximal effective concentration (EC₅₀) of apoptosis
by monospecific vs bispecific MBs on Daudi cells

| Molecule | | EC₅₀ (nM) |
|---|---|---|
| CD37 monospecific | Naratuximab | 0.042 |
| MBs | AGS67C | 0.134 |
| | 016-H5L2 | 0.065 |
| CD37/NKp46 | Naratuximab × NKp46-1 H1L1 | 0.054 |
| bispecific MBs | Naratuximab × NKp46-2 H1L1 | 0.032 |
| | Naratuximab × NKp46-4 H1L2 | 0.073 |
| | Naratuximab × NKp46 09 | — |
| | AGS67C × NKp46-1 H1L1 | 0.077 |
| | AGS67C × NKp46-2 H1L1 | — |
| | AGS67C × NKp46-4 H1L2 | — |
| | AGS67C × NKp46 09 | — |
| | 016-H5L2 × NKp46-1 H1L1 | 0.102 |
| | 016-H5L2 × NKp46-2 H1L1 | — |
| | 016-H5L2 × NKp46-4 H1L2 | 0.142 |
| | 016-H5L2 × NKp46 09 | — |

### EQUIVALENTS/OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

1. A fusion polypeptide comprising: (1) a hematopoietic cell-binding moiety and (2) a nanocage monomer or subunit thereof.

2. The fusion polypeptide of claim 1, wherein the hematopoietic cell-binding moiety comprises an antibody or antigen-binding fragment thereof.

3. The fusion polypeptide of claim 2, wherein the antibody or antigen-binding fragment thereof comprises an immunoglobulin heavy chain variable region (V_{H}) and an immunoglobulin light chain variable region (V_{L} or V_{K}).

4. The fusion polypeptide of claim 3, wherein the antibody or antigen-binding fragment thereof comprises a Fab fragment.

5. The fusion polypeptide of claim 4, wherein the Fab fragment is a single-chain Fab fragment (scFab).

6. The fusion polypeptide of any one of claims 1-5, comprising a lymphoid cell-binding moiety.

7. The fusion polypeptide of claim 6, wherein the lymphoid cell-binding moiety is a natural killer cell-binding moiety or T cell-binding moiety.

8. The fusion polypeptide of claim 7, wherein the lymphoid cell-binding moiety is a natural killer cell-binding moiety.

9. The fusion polypeptide of claim 8, wherein the natural killer cell-binding moiety is a CD16a-binding moiety, a NKp46-binding moiety, or a NKG2D-binding moiety.

10. The fusion polypeptide of claim 9, wherein the natural killer cell-binding moiety comprises an antibody fragment comprising a heavy chain variable region (V_{H}) and light chain variable region (V_{L} or V_{K}), wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a CD16a, NKp46, or NKG2D antibody.

11. The fusion polypeptide of claim 10, wherein the CDRs of the V_{H} and the V_{L} or V_{K} have sequences which are identical to those of the heavy and light chain CDRs of a CD16a, NKp46, or NKG2D antibody, except for one or two amino acid substitutions total across all six CDRs.

12. The fusion polypeptide of claim 11, wherein the CDRs of the V_{H} and the V_{L} or V_{K} are identical to those of the heavy and light chain CDRs of a CD16a, NKp46, or NKG2D antibody.

13. The fusion polypeptide of claim 9, wherein the natural killer cell-binding moiety comprises an antibody fragment comprising heavy and light chain variable regions with sequences that have at least 85% identity to those of the heavy and light chain variable regions of a CD16a, NKp46, or NKG2D antibody.

14. The fusion polypeptide of claim 7, wherein the lymphoid cell-binding moiety is a T cell-binding moiety.

15. The fusion polypeptide of claim 14, wherein the T cell-binding moiety is a CD3-binding moiety.

16. The fusion polypeptide of claim 15, wherein the T cell-binding moiety comprises an antibody fragment comprising a heavy chain variable region (V_{H}) and light chain variable region (V_{L} or V_{K}),
wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a CD3 antibody.

17. The fusion polypeptide of claim 16, wherein the CDRs of the V_{H} and the V_{L} or V_{K} have sequences which are identical to those of the heavy and light chain CDRs of a CD3 antibody, except for one or two amino acid substitutions total across all six CDRs.

18. The fusion polypeptide of claim 17, wherein the CDRs of the V_{H} and the V_{L} or V_{K} are identical to those of the heavy and light chain CDRs of a CD3 antibody.

19. The fusion polypeptide of claim 15, wherein the T cell-binding moiety comprises an antibody fragment comprising heavy and light chain variable regions with sequences that have at least 85% identity to those of the heavy and light chain variable regions of a CD3 antibody.

20. The fusion polypeptide of any one of claims 1-5, comprising a myeloid cell-binding moiety.

21. The fusion polypeptide of claim 20, wherein the myeloid cell-binding moiety is a SIRPα-binding moiety.

22. The fusion polypeptide of claim 21, wherein the myeloid cell-binding moiety comprises an antibody fragment comprising a heavy chain variable region (V_{H}) and light chain variable region (V_{L} or V_{K}),
wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a SIRPα antibody.

23. The fusion polypeptide of claim 22, wherein the CDRs of the V_{H} and the V_{L} or V_{K} have sequences which are identical to those of the heavy and light chain CDRs of a SIRPα antibody, except for one or two amino acid substitutions total across all six CDRs.

24. The fusion polypeptide of claim 23, wherein the CDRs of the V_{H} and the V_{L} or V_{K} are identical to those of the heavy and light chain CDRs of a SIRPα antibody.

25. The fusion polypeptide of claim 21, wherein the natural killer cell-binding moiety comprises an antibody fragment comprising heavy and light chain variable regions with sequences that have at least 85% identity to those of the heavy and light chain variable regions of a SIRPα antibody.

26. The fusion polypeptide of any one of claims 1-25, wherein the nanocage monomer is a ferritin monomer or a subunit thereof.

27. The fusion polypeptide of claim 26, wherein the ferritin monomer is a human ferritin monomer.

28. The fusion polypeptide of claim 26 or 27, wherein the ferritin monomer is a ferritin light chain.

29. The fusion polypeptide of any one of claims 1-28, wherein the hematopoietic cell-binding moiety is linked via an amino acid linker to the nanocage monomer or subunit thereof.

30. The fusion polypeptide of any one of claims 1-29, wherein the hematopoietic cell-binding moiety is linked via the N-terminus of the nanocage monomer or subunit thereof.

31. A self-assembled polypeptide complex comprising:
(a) a plurality of hematopoietic cell-binding fusion polypeptides, each hematopoietic cell-binding fusion polypeptide being a fusion polypeptide of any one of claims 1-30; and
(b) a plurality of Fc fusion polypeptides, each Fc fusion polypeptide comprising (1) an Fc polypeptide and (2) a nanocage monomer or subunit thereof.

32. The self-assembled polypeptide complex of claim 31, further comprising
(c) a plurality of tumor-binding fusion polypeptides, each tumor-binding fusion polypeptide comprising (1) tumor-binding moiety and (2) a nanocage monomer or subunit thereof.

33. The self-assembled polypeptide complex of claim 31 or 32, wherein, within each Fc fusion polypeptide, the Fc polypeptide is linked via an amino acid linker to the nanocage monomer or subunit thereof.

34. The self-assembled polypeptide complex of claim 33, wherein the Fc polypeptide is linked via the N-terminus of the nanocage monomer or subunit thereof.

35. The self-assembled polypeptide complex of claim 33, wherein the Fc polypeptide is linked via the C-terminus of the nanocage monomer or subunit thereof.

36. The self-assembled polypeptide complex of any one of claims 32-36, wherein, within each tumor-binding fusion polypeptide, the tumor-binding moiety is linked via an amino acid linker to the nanocage monomer or subunit thereof.

37. The self-assembled polypeptide complex of claim 36, wherein the tumor-binding moiety is linked via the N-terminus of the nanocage monomer or subunit thereof.

38. A self-assembled polypeptide complex comprising:
(a) a plurality of hematopoietic cell-binding fusion polypeptides, each hematopoietic cell-binding fusion polypeptide being a fusion polypeptide of any one of claims 1-30; and
(b) a plurality of Fc fusion polypeptides, each Fc fusion polypeptide comprising (1) an Fc polypeptide linked via an amino acid linker to the C-terminus of (2) a nanocage monomer or subunit thereof.

39. A self-assembled polypeptide complex comprising:
(a) a plurality of tumor-binding fusion polypeptides, each tumor-binding fusion polypeptide comprising (1) tumor-binding moiety linked via an amino acid linker to (2) a nanocage monomer or subunit thereof; and
(b) a plurality of Fc fusion polypeptides, each Fc fusion polypeptide comprising (1) an Fc polypeptide linked via an amino acid linker to the C-terminus of (2) a nanocage monomer or subunit thereof.

40. A self-assembled polypeptide complex comprising:
(a) a plurality of hematopoietic cell-binding fusion polypeptides, each hematopoietic cell-binding fusion polypeptide being a fusion polypeptide of any one of claims 1-30; and
(b) a plurality of tumor-binding fusion polypeptides, each tumor-binding fusion polypeptide comprising (1) tumor-binding moiety linked via an amino acid linker to (2) a nanocage monomer or subunit thereof; and
(c) a plurality of Fc fusion polypeptides, each Fc fusion polypeptide comprising (1) an Fc polypeptide linked to the C-terminus of (2) a nanocage monomer or subunit thereof.

41. The self-assembling polypeptide complex of any one of claims 39-41, wherein the Fc polypeptide is linked to the C-terminus of a C-half nanocage monomer.

42. The self-assembling polypeptide complex of claim 41, wherein the Fc polypeptide is linked to the C-terminus of a C-half ferritin.

43. The self-assembled polypeptide complex of any one of claim 32-37 or 39-43, wherein the tumor-binding moiety comprises an antibody or antigen-binding fragment thereof.

44. The self-assembled polypeptide complex of claim 43, wherein the antibody or antigen-binding fragment thereof comprises an immunoglobulin heavy chain variable region (V_{H}) and an immunoglobulin light chain variable region (V_{L} or V_{K}).

45. The self-assembled polypeptide complex of claim 44, wherein the antibody or antigen-binding fragment thereof comprises a Fab fragment.

46. The self-assembled polypeptide complex of claim 45, wherein the Fab fragment is a single-chain Fab fragment (scFab).

47. The self-assembled polypeptide complex of any one of claims 43-46, wherein the tumor-binding moiety is a CD20-binding moiety or a CD37-binding moiety.

48. The self-assembled polypeptide complex of claim 47, wherein the tumor-binding moiety comprises an antibody fragment comprising a heavy chain variable region (V_{H}) and light chain variable region (V_{L} or V_{K}),
wherein the CDRs of the V_{H} and the V_{L} or V_{K} (CDR-H1, CDR-H2, CDR-H3, CDR-L1, CDR-L2, and CDR-L3) have sequences which each differ by at most two amino acids from the CDRs of a CD20 or CD37 antibody.

49. The self-assembled polypeptide complex of claim 48, wherein the CDRs of the V_{H} and the V_{L} or V_{K} have sequences which are identical to those of the heavy and light chain CDRs of a CD20 or CD37 antibody, except for one or two amino acid substitutions total across all six CDRs.

50. The self-assembled polypeptide complex of claim 49, wherein the CDRs of the V_{H} and the V_{L} or V_{K} are identical to those of the heavy and light chain CDRs of a CD20 or CD37 antibody.

51. The self-assembled polypeptide complex of claim 47, wherein the natural killer cell-binding moiety comprises an antibody fragment comprising heavy and light chain variable regions with sequences that have at least 85% identity to those of the heavy and light chain variable regions of a CD20 or CD37 antibody.

52. The self-assembled polypeptide complex of any one of claims 31-51, wherein the nanocage monomers within the Fc fusion polypeptide and within hematopoietic cell-binding fusion polypeptide and/or the tumor-binding fusion polypeptide are each a ferritin monomer or a subunit thereof.

53. The self-assembled polypeptide complex of claim 52, wherein the ferritin monomer is a human ferritin monomer.

54. The self-assembled polypeptide complex of claim 52 or 53, wherein the ferritin monomer is a ferritin light chain.

55. The self-assembled polypeptide complex of claim 54, which does not comprise any ferritin heavy chains or subunits of ferritin heavy chains.

56. The self-assembled polypeptide complex of any one of claims 52-55, which does not comprise any iron-binding moieties.

57. A pharmaceutical composition comprising a self-assembled polypeptide complex of any one of claims 31-56 and a pharmaceutically acceptable excipient.

58. A method of treating, ameliorating, or preventing a disease or condition, the method comprising administering to a subject the self-assembled polypeptide complex of any of claims 31-56 or the pharmaceutical composition of claim 57.

59. The method of claim 58, wherein the subject is a mammal.

60. The method of claim 59, wherein the subject is human.

61. The method of claim 60, wherein the disease or condition is cancer.

62. The method of claim 61, wherein the cancer is a B-cell lymphoma.

63. A fusion polypeptide comprising a contiguous full-length ferritin light chain having an N-terminus and a C-terminus, and
(a) an antibody fragment fused via a first amino acid linker to the N-terminus and an Fc polypeptide fused via a second amino acid linker to the C-terminus; or
(b) an antibody fragment fused via a first amino acid linker to the C-terminus and an Fc polypeptide fused via a second amino acid linker to the N-terminus.

64. The fusion polypeptide of claim 63, wherein the antibody fragment is fused via a first amino acid linker to the N-terminus and an Fc polypeptide fused via a second amino acid linker to the C-terminus.

65. The fusion polypeptide of claim 63 or 64, wherein the antibody fragment comprises an immunoglobulin heavy chain variable region (V_{H}) and an immunoglobulin light chain variable region (V_{L} or V_{K}).

66. The fusion polypeptide of claim 65, wherein the antibody or antigen-binding fragment thereof comprises a Fab fragment.

67. The fusion polypeptide of claim 66, wherein the Fab fragment is a single-chain Fab fragment (scFab).

68. The fusion polypeptide of any one of claims 63-67, wherein the antibody fragment is capable of binding to a tumor-associated antigen.

69. A self-assembled polypeptide complex comprising a fusion polypeptide of any one of claims 63-68.

70. A pharmaceutical composition comprising the self-assembled polypeptide complex of claim 69 and a pharmaceutically acceptable excipient.

71. A method of treating, ameliorating, or preventing a disease or condition, the method comprising administering to a subject the self-assembled polypeptide complex of claim 69 or the pharmaceutical composition of claim 70.

72. A method comprising a step of contacting a tumor cell with a self-assembled polypeptide complex of any one of claims 32-37 and 39-56.

73. The method of claim 72, wherein the step of contacting results in apoptosis of the tumor cell.

74. The method of claim 73, wherein the tumor cell expresses CD37 and the self-assembled polypeptide complex comprises a fusion protein comprising an α-CD37 Fab.

75. The method of claim 73 or 74, wherein apoptosis is induced with an EC₅₀ value of less than 1 nM for the self-assembled polypeptide complex.

76. The method of any one of claims 72-75, wherein the step of contacting results in antibody-dependent cellular cytotoxicity (ADCC) of the tumor cell.

77. The method of claim 76, wherein ADCC is induced with an EC₅₀ value of less than 1 nM for the self-assembled polypeptide complex.

78. The method of any one of claims 72-77, wherein the step of contacting is conducted in the presence of complement proteins and results in complement-dependent cytotoxicity (CDC) of the tumor cell.

79. The method of claim 78, wherein CDC is induced with an EC₅₀ value of less than 0.4 nM for the self-assembled polypeptide complex.

80. The method of any one of claims 72-79, wherein the step of contacting results in antibody-dependent cellular phagocytosis (ADCP) of the tumor cell.

81. The method of claim 80, wherein ADCP is induced with an EC₅₀ value of less than 0.1 nM for the self-assembled polypeptide complex.

82. The method of any one of claims 72-80, wherein the step of contacting occurs inside a mammal having a tumor.

83. The method of claim 82, wherein the mammal is human.

84. The self-assembled polypeptide complex of any one of claims 32-37 and 39-56, wherein the self-assembled polypeptide complex, when contacted with a tumor cell, is capable of inducing an effect selected from the group consisting of
(a) apoptosis of the tumor cell with an EC₅₀ value of less than 1 nM;
(b) ADCC of the tumor cell with an EC₅₀ value of less than 1 nM;
(c) CDC of the tumor cell, when the self-assembled polypeptide complex is contacted with the tumor cell in the presence of complement, with an EC₅₀ value of less than 0.4 nM;
(d) ADCP with an EC₅₀ value of less than 0.1 nM; and
(e) any combination of the foregoing.

85. Use of the self-assembled polypeptide complex of any one of claim 31-56 or 69 or the pharmaceutical composition of claim 57 or 70 to treat, ameliorate, or prevent a disease or condition in a subject.

86. The use of claim 85, wherein the subject is a mammal.

87. The use of claim 86, wherein the subject is human.

88. The use of any one of claims 85-87, wherein the disease or condition is cancer.

89. The use of claim 88, wherein the cancer is a B-cell lymphoma.


SEQUENCE LISTING (435 sequences)

SEQ ID NO: 1 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLRHD

SEQ ID NO: 2 - protein, 90 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEW

SEQ ID NO: 3 - protein, 85 aa, Homo sapiens
GKTPDAMKAAMALEKKLNQALLDLHALGSARTDPHLCDFLETHFLDEEVKLIKKMGDHLTNLHRLGGPEAGLGEYLFERLTLRHD

SEQ ID NO: 4 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 524 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 4 aa, synthetic construct
GGGS

SEQ ID NO: 7 - protein, 5 aa, synthetic construct
GGGGS

SEQ ID NO: 8 - protein, 120 aa, synthetic construct
QVQLVQSGAEVKKPGESLKVSCKASGYTFTSYYMHWVRQAPGQGLEWMGAIEPMYGSTSYAQKFQGRVTMTRDTSTSTVYMELSSLRSEDTAVYYCARGSAYYYDFADYWGQGTLVTVSS

SEQ ID NO: 9 - protein, 106 aa, synthetic construct
SYVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 10 - protein, 5 aa, synthetic construct
SYYMH

SEQ ID NO: 11 - protein, 17 aa, synthetic construct
AIEPMYGSTSYAQKFQG

SEQ ID NO: 12 - protein, 11 aa, synthetic construct
GSAYYYDFADY

SEQ ID NO: 13 - protein, 11 aa, synthetic construct
GGHNIGSKNVH

SEQ ID NO: 14 - protein, 7 aa, synthetic construct
QDNKRPS

SEQ ID NO: 15 - protein, 9 aa, synthetic construct
QVWDNYSVL

SEQ ID NO: 16 - protein, 8 aa, synthetic construct
GYTFTSYY

SEQ ID NO: 17 - protein, 8 aa, synthetic construct
IEPMYGST

SEQ ID NO: 18 - protein, 13 aa, synthetic construct
ARGSAYYYDFADY

SEQ ID NO: 19 - protein, 6 aa, synthetic construct
NIGSKN

SEQ ID NO: 20 - protein, 9 aa, synthetic construct
QVWDNYSVL

SEQ ID NO: 21 - protein, 7 aa, synthetic construct
GYTFTSY

SEQ ID NO: 22 - protein, 6 aa, synthetic construct
EPMYGS

SEQ ID NO: 23 - protein, 11 aa, synthetic construct
GSAYYYDFADY

SEQ ID NO: 24 - protein, 11 aa, synthetic construct
GGHNIGSKNVH

SEQ ID NO: 25 - protein, 7 aa, synthetic construct
QDNKRPS

SEQ ID NO: 26 - protein, 9 aa, synthetic construct
QVWDNYSVL

SEQ ID NO: 27 - protein, 120 aa, synthetic construct
QVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSS

SEQ ID NO: 28 - protein, 106 aa, synthetic construct; identical to SEQ ID NO: 9

SEQ ID NO: 29 - protein, 5 aa, synthetic construct
SYYMH

SEQ ID NO: 30 - protein, 17 aa, synthetic construct
IINPSGGSTSYAQKFQG

SEQ ID NO: 31 - protein, 11 aa, synthetic construct
GSAYYYDFADY

SEQ ID NO: 32 - protein, 11 aa, synthetic construct
GGHNIGSKNVH

SEQ ID NO: 33 - protein, 7 aa, synthetic construct
QDNKRPS

SEQ ID NO: 34 - protein, 9 aa, synthetic construct
QVWDNYSVL

SEQ ID NO: 35 - protein, 8 aa, synthetic construct
GYTFTSYY

SEQ ID NO: 36 - protein, 8 aa, synthetic construct
INPSGGST

SEQ ID NO: 37 - protein, 13 aa, synthetic construct
ARGSAYYYDFADY

SEQ ID NO: 38 - protein, 6 aa, synthetic construct
NIGSKN

SEQ ID NO: 39 - protein, 9 aa, synthetic construct
QVWDNYSVL

SEQ ID NO: 40 - protein, 7 aa, synthetic construct
GYTFTSY

SEQ ID NO: 41 - protein, 6 aa, synthetic construct
NPSGGS

SEQ ID NO: 42 - protein, 11 aa, synthetic construct
GSAYYYDFADY

SEQ ID NO: 43 - protein, 11 aa, synthetic construct
GGHNIGSKNVH

SEQ ID NO: 44 - protein, 7 aa, synthetic construct
QDNKRPS

SEQ ID NO: 45 - protein, 9 aa, synthetic construct
QVWDNYSVL

SEQ ID NO: 46 - protein, 120 aa, synthetic construct
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSS

SEQ ID NO: 47 - protein, 107 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 48 - protein, 5 aa, synthetic construct
DYVIN

SEQ ID NO: 49 - protein, 17 aa, synthetic construct
EIYPGSGTNYYNEKFKA

SEQ ID NO: 50 - protein, 11 aa, synthetic construct
RGRYGLYAMDY

SEQ ID NO: 51 - protein, 11 aa, synthetic construct
RASQDISNYLN

SEQ ID NO: 52 - protein, 7 aa, synthetic construct
YTSRLHS

SEQ ID NO: 53 - protein, 9 aa, synthetic construct
QQGNTRPWT

SEQ ID NO: 54 - protein, 8 aa, synthetic construct
GYTFSDYV

SEQ ID NO: 55 - protein, 8 aa, synthetic construct
IYPGSGTN

SEQ ID NO: 56 - protein, 13 aa, synthetic construct
ARRGRYGLYAMDY

SEQ ID NO: 57 - protein, 6 aa, synthetic construct
QDISNY

SEQ ID NO: 58 - protein, 9 aa, synthetic construct
QQGNTRPWT

SEQ ID NO: 59 - protein, 7 aa, synthetic construct
GYTFSDY

SEQ ID NO: 60 - protein, 6 aa, synthetic construct
YPGSGT

SEQ ID NO: 61 - protein, 11 aa, synthetic construct
RGRYGLYAMDY

SEQ ID NO: 62 - protein, 11 aa, synthetic construct
RASQDISNYLN

SEQ ID NO: 63 - protein, 7 aa, synthetic construct
YTSRLHS

SEQ ID NO: 64 - protein, 9 aa, synthetic construct
QQGNTRPWT

SEQ ID NO: 65 - protein, 116 aa, synthetic construct
QVQLVQSGAEVKKPGSSVKVSCKASGYTFSEYTMHWVRQAPGQGLEWMGGISPNIGGTSYNQKFKGRVTITADKSTSTAYMELSSLRSEDTAVYYCARRGGSFDYWGQGTTVTVSS

SEQ ID NO: 66 - protein, 107 aa, synthetic construct
EIVMTQSPATLSVSPGERATLSCRASQSISDYLHWYQQKPGQAPRLLIKYASQSISGIPARFSGSGSGTDFTLTISSLEPEDFAVYYCQNGHSFPLTFGQGTKLEIK

SEQ ID NO: 67 - protein, 5 aa, synthetic construct
EYTMH

SEQ ID NO: 68 - protein, 17 aa, synthetic construct
GISPNIGGTSYNQKFKG

SEQ ID NO: 69 - protein, 7 aa, synthetic construct
RGGSFDY

SEQ ID NO: 70 - protein, 11 aa, synthetic construct
RASQSISDYLH

SEQ ID NO: 71 - protein, 7 aa, synthetic construct
YASQSIS

SEQ ID NO: 72 - protein, 9 aa, synthetic construct
QNGHSFPLT

SEQ ID NO: 73 - protein, 8 aa, synthetic construct
GYTFSEYT

SEQ ID NO: 74 - protein, 8 aa, synthetic construct
ISPNIGGT

SEQ ID NO: 75 - protein, 9 aa, synthetic construct
ARRGGSFDY

SEQ ID NO: 76 - protein, 6 aa, synthetic construct
QSISDY

SEQ ID NO: 77 - protein, 9 aa, synthetic construct
QNGHSFPLT

SEQ ID NO: 78 - protein, 7 aa, synthetic construct
GYTFSEY

SEQ ID NO: 79 - protein, 6 aa, synthetic construct
SPNIGG

SEQ ID NO: 80 - protein, 7 aa, synthetic construct
RGGSFDY

SEQ ID NO: 81 - protein, 11 aa, synthetic construct
RASQSISDYLH

SEQ ID NO: 82 - protein, 7 aa, synthetic construct
YASQSIS

SEQ ID NO: 83 - protein, 9 aa, synthetic construct
QNGHSFPLT

SEQ ID NO: 84 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 85 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 86 - protein, 5 aa, synthetic construct
SYSMN

SEQ ID NO: 87 - protein, 17 aa, synthetic construct
SISSSSSYIYYADSVKG

SEQ ID NO: 88 - protein, 13 aa, synthetic construct
GAPMGAAAGWFDP

SEQ ID NO: 89 - protein, 11 aa, synthetic construct
RASQGISSWLA

SEQ ID NO: 90 - protein, 7 aa, synthetic construct
AASSLQS

SEQ ID NO: 91 - protein, 9 aa, synthetic construct
QQGVSFPRT

SEQ ID NO: 92 - protein, 8 aa, synthetic construct
GFTFSSYS

SEQ ID NO: 93 - protein, 8 aa, synthetic construct
ISSSSSYI

SEQ ID NO: 94 - protein, 15 aa, synthetic construct
ARGAPMGAAAGWFDP

SEQ ID NO: 95 - protein, 6 aa, synthetic construct
QGISSW

SEQ ID NO: 96 - protein, 9 aa, synthetic construct
QQGVSFPRT

SEQ ID NO: 97 - protein, 7 aa, synthetic construct
GFTFSSY

SEQ ID NO: 98 - protein, 6 aa, synthetic construct
SSSSSY

SEQ ID NO: 99 - protein, 13 aa, synthetic construct
GAPMGAAAGWFDP

SEQ ID NO: 100 - protein, 11 aa, synthetic construct
RASQGISSWLA

SEQ ID NO: 101 - protein, 7 aa, synthetic construct
AASSLQS

SEQ ID NO: 102 - protein, 9 aa, synthetic construct
QQGVSFPRT

SEQ ID NO: 103 - protein, 126 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 104 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 105 - protein, 5 aa, synthetic construct
SYYMH

SEQ ID NO: 106 - protein, 17 aa, synthetic construct
IINPSGGSTSYAQKFQG

SEQ ID NO: 107 - protein, 17 aa, synthetic construct
GAPNYGDTTHDYYYMDV

SEQ ID NO: 108 - protein, 11 aa, synthetic construct
RASQSVSSNLA

SEQ ID NO: 109 - protein, 7 aa, synthetic construct
GASTRAT

SEQ ID NO: 110 - protein, 9 aa, synthetic construct
QQYDDWPFT

SEQ ID NO: 111 - protein, 8 aa, synthetic construct
GYTFTSYY

SEQ ID NO: 112 - protein, 8 aa, synthetic construct
INPSGGST

SEQ ID NO: 113 - protein, 19 aa, synthetic construct
ARGAPNYGDTTHDYYYMDV

SEQ ID NO: 114 - protein, 6 aa, synthetic construct
QSVSSN

SEQ ID NO: 115 - protein, 9 aa, synthetic construct
QQYDDWPFT

SEQ ID NO: 116 - protein, 7 aa, synthetic construct
GYTFTSY

SEQ ID NO: 117 - protein, 6 aa, synthetic construct
NPSGGS

SEQ ID NO: 118 - protein, 17 aa, synthetic construct
GAPNYGDTTHDYYYMDV

SEQ ID NO: 119 - protein, 11 aa, synthetic construct
RASQSVSSNLA

SEQ ID NO: 120 - protein, 7 aa, synthetic construct
GASTRAT

SEQ ID NO: 121 - protein, 9 aa, synthetic construct
QQYDDWPFT

SEQ ID NO: 122 - protein, 116 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSSA

SEQ ID NO: 123 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKR

SEQ ID NO: 124 - protein, 5 aa, synthetic construct
SYYWS

SEQ ID NO: 125 - skipped

SEQ ID NO: 126 - protein, 7 aa, synthetic construct
WDDAFNI

SEQ ID NO: 127 - protein, 12 aa, synthetic construct
RASQSVSSSYLA

SEQ ID NO: 128 - protein, 7 aa, synthetic construct
GASSRAT

SEQ ID NO: 129 - protein, 9 aa, synthetic construct
QQYGSSPWT

SEQ ID NO: 130 - protein, 8 aa, synthetic construct
DDSISSYY

SEQ ID NO: 131 - protein, 7 aa, synthetic construct
ISYSGSA

SEQ ID NO: 132 - protein, 9 aa, synthetic construct
ANWDDAFNI

SEQ ID NO: 133 - protein, 7 aa, synthetic construct
QSVSSSY

SEQ ID NO: 134 - protein, 9 aa, synthetic construct
QQYGSSPWT

SEQ ID NO: 135 - protein, 7 aa, synthetic construct
DDSISSY

SEQ ID NO: 136 - protein, 5 aa, synthetic construct
SYSGS

SEQ ID NO: 137 - protein, 7 aa, synthetic construct
WDDAFNI

SEQ ID NO: 138 - protein, 12 aa, synthetic construct
RASQSVSSSYLA

SEQ ID NO: 139 - protein, 7 aa, synthetic construct
GASSRAT

SEQ ID NO: 140 - protein, 9 aa, synthetic construct
QQYGSSPWT

SEQ ID NO: 141 - protein, 120 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSA

SEQ ID NO: 142 - protein, 113 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPS

SEQ ID NO: 143 - protein, 5 aa, synthetic construct
RYTMH

SEQ ID NO: 144 - protein, 17 aa, synthetic construct
YINPSRGYTNYNQKVKD

SEQ ID NO: 145 - protein, 10 aa, synthetic construct
YYDDHYCLDY

SEQ ID NO: 146 - protein, 10 aa, synthetic construct
SASSSVSYMN

SEQ ID NO: 147 - protein, 7 aa, synthetic construct
DTSKLAS

SEQ ID NO: 148 - protein, 9 aa, synthetic construct
QQWSSNPFT

SEQ ID NO: 149 - protein, 8 aa, synthetic construct
GYTFTRYT

SEQ ID NO: 150 - protein, 8 aa, synthetic construct
INPSRGYT

SEQ ID NO: 151 - protein, 12 aa, synthetic construct
ARYYDDHYCLDY

SEQ ID NO: 152 - protein, 5 aa, synthetic construct
SSVSY

SEQ ID NO: 153 - protein, 9 aa, synthetic construct
QQWSSNPFT

SEQ ID NO: 154 - protein, 7 aa, synthetic construct
GYTFTRY

SEQ ID NO: 155 - protein, 6 aa, synthetic construct
NPSRGY

SEQ ID NO: 156 - protein, 10 aa, synthetic construct
YYDDHYCLDY

SEQ ID NO: 157 - protein, 10 aa, synthetic construct
SASSSVSYMN

SEQ ID NO: 158 - protein, 7 aa, synthetic construct
DTSKLAS

SEQ ID NO: 159 - protein, 9 aa, synthetic construct
QQWSSNPFT

SEQ ID NO: 160 - protein, 120 aa, synthetic construct
QVQLVQSGAEVKKPGASVKVSCKASGYTFTSYWITWVKQAPGQGLEWIGDIYPGSGSTNHIEKFKSKATLTVDTSISTAYMELSRLRSDDTAVYYCATGYGSSYGYFDYWGQGTLVTVSS

SEQ ID NO: 161 - protein, 107 aa, synthetic construct
DIQMTQSPSSLSASVGDRVTITCRASENIYSYLAWYQQKPGKAPKLLIYTAKTLAEGVPSRFSGSGSGTDFTLTISSLQPEDFATYYCQHQYGPPFTFGQGTKLEIK

SEQ ID NO: 162 - protein, 5 aa, synthetic construct
SYWIT

SEQ ID NO: 163 - protein, 17 aa, synthetic construct
DIYPGSGSTNHIEKFKS

SEQ ID NO: 164 - protein, 11 aa, synthetic construct
GYGSSYGYFDY

SEQ ID NO: 165 - protein, 11 aa, synthetic construct
RASENIYSYLA

SEQ ID NO: 166 - protein, 7 aa, synthetic construct
TAKTLAE

SEQ ID NO: 167 - protein, 9 aa, synthetic construct
QHQYGPPFT

SEQ ID NO: 168 - protein, 8 aa, synthetic construct
GYTFTSYW

SEQ ID NO: 169 - protein, 8 aa, synthetic construct
IYPGSGST

SEQ ID NO: 170 - protein, 13 aa, synthetic construct
ATGYGSSYGYFDY

SEQ ID NO: 171 - protein, 6 aa, synthetic construct
ENIYSY

SEQ ID NO: 172 - protein, 9 aa, synthetic construct
QHQYGPPFT

SEQ ID NO: 173 - protein, 7 aa, synthetic construct
GYTFTSY

SEQ ID NO: 174 - protein, 6 aa, synthetic construct
YPGSGS

SEQ ID NO: 175 - protein, 11 aa, synthetic construct
GYGSSYGYFDY

SEQ ID NO: 176 - protein, 11 aa, synthetic construct
RASENIYSYLA

SEQ ID NO: 177 - protein, 7 aa, synthetic construct
TAKTLAE

SEQ ID NO: 178 - protein, 9 aa, synthetic construct
QHQYGPPFT

SEQ ID NO: 179 - protein, 119 aa, synthetic construct
QVQLVQSGAEVKKPGASVKVSCKASGYTFTSYWMHWVRQAPGQGLEWMGNIDPSDSDTHYNQKFKDRVTMTRDTSTSTVYMELSSLRSEDTAVYYCARGYSKYYAMDYWGQGTLVTVSS

SEQ ID NO: 180 - protein, 112 aa, synthetic construct
DIVMTQTPLSLSVTPGQPASISCRSSQSIVHSYGNTYLEWYLQKPGQSPQLLIYKVSNRFSGVPDRFSGSGSGTDFTLKISRVEAEDVGVYYCFQGSHVPYTFGQGTKLEIK

SEQ ID NO: 181 - protein, 5 aa, synthetic construct
SYWMH

SEQ ID NO: 182 - protein, 17 aa, synthetic construct
NIDPSDSDTHYNQKFKD

SEQ ID NO: 183 - protein, 10 aa, synthetic construct
GYSKYYAMDY

SEQ ID NO: 184 - protein, 16 aa, synthetic construct
RSSQSIVHSYGNTYLE

SEQ ID NO: 185 - protein, 7 aa, synthetic construct
KVSNRFS

SEQ ID NO: 186 - protein, 9 aa, synthetic construct
FQGSHVPYT

SEQ ID NO: 187 - protein, 8 aa, synthetic construct
GYTFTSYW

SEQ ID NO: 188 - protein, 8 aa, synthetic construct
IDPSDSDT

SEQ ID NO: 189 - protein, 12 aa, synthetic construct
ARGYSKYYAMDY

SEQ ID NO: 190 - protein, 11 aa, synthetic construct
QSIVHSYGNTY

SEQ ID NO: 191 - protein, 9 aa, synthetic construct
FQGSHVPYT

SEQ ID NO: 192 - protein, 7 aa, synthetic construct
GYTFTSY

SEQ ID NO: 193 - protein, 6 aa, synthetic construct
DPSDSD

SEQ ID NO: 194 - protein, 10 aa, synthetic construct
GYSKYYAMDY

SEQ ID NO: 195 - protein, 16 aa, synthetic construct
RSSQSIVHSYGNTYLE

SEQ ID NO: 196 - protein, 7 aa, synthetic construct
KVSNRFS

SEQ ID NO: 197 - protein, 9 aa, synthetic construct
FQGSHVPYT

SEQ ID NO: 198 - protein, 122 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAA

SEQ ID NO: 199 - protein, 107 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKR

SEQ ID NO: 200 - protein, 5 aa, synthetic construct
SYNMH

SEQ ID NO: 201 - protein, 17 aa, synthetic construct
AIYPGNGDTSYNQKFKG

SEQ ID NO: 202 - protein, 12 aa, synthetic construct
STYYGGDWYFNV

SEQ ID NO: 203 - protein, 10 aa, synthetic construct
RASSSVSYIH

SEQ ID NO: 204 - protein, 7 aa, synthetic construct
ATSNLAS

SEQ ID NO: 205 - protein, 9 aa, synthetic construct
QQWTSNPPT

SEQ ID NO: 206 - protein, 8 aa, synthetic construct
GYTFTSYN

SEQ ID NO: 207 - protein, 8 aa, synthetic construct
IYPGNGDT

SEQ ID NO: 208 - protein, 14 aa, synthetic construct
ARSTYYGGDWYFNV

SEQ ID NO: 209 - protein, 5 aa, synthetic construct
SSVSY

SEQ ID NO: 210 - protein, 9 aa, synthetic construct
QQWTSNPPT

SEQ ID NO: 211 - protein, 7 aa, synthetic construct
GYTFTSY

SEQ ID NO: 212 - protein, 6 aa, synthetic construct
YPGNGD

SEQ ID NO: 213 - protein, 12 aa, synthetic construct
STYYGGDWYFNV

SEQ ID NO: 214 - protein, 10 aa, synthetic construct
RASSSVSYIH

SEQ ID NO: 215 - protein, 7 aa, synthetic construct
ATSNLAS

SEQ ID NO: 216 - protein, 9 aa, synthetic construct
QQWTSNPPT

SEQ ID NO: 217 - protein, 123 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSA

SEQ ID NO: 218 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKR

SEQ ID NO: 219 - protein, 5 aa, synthetic construct
DYAMH

SEQ ID NO: 220 - protein, 17 aa, synthetic construct
TISWNSGSIGYADSVKG

SEQ ID NO: 221 - protein, 13 aa, synthetic construct
DIQYGNYYYGMDV

SEQ ID NO: 222 - protein, 11 aa, synthetic construct
RASQSVSSYLA

SEQ ID NO: 223 - protein, 7 aa, synthetic construct
DASNRAT

SEQ ID NO: 224 - protein, 9 aa, synthetic construct
QQRSNWPIT

SEQ ID NO: 225 - protein, 8 aa, synthetic construct
GFTFNDYA

SEQ ID NO: 226 - protein, 8 aa, synthetic construct
ISWNSGSI

SEQ ID NO: 227 - protein, 15 aa, synthetic construct
AKDIQYGNYYYGMDV

SEQ ID NO: 228 - protein, 6 aa, synthetic construct
QSVSSY

SEQ ID NO: 229 - protein, 9 aa, synthetic construct
QQRSNWPIT

SEQ ID NO: 230 - protein, 7 aa, synthetic construct
GFTFNDY

SEQ ID NO: 231 - protein, 6 aa, synthetic construct
SWNSGS

SEQ ID NO: 232 - protein, 13 aa, synthetic construct
DIQYGNYYYGMDV

SEQ ID NO: 233 - protein, 11 aa, synthetic construct
RASQSVSSYLA

SEQ ID NO: 234 - protein, 7 aa, synthetic construct
DASNRAT

SEQ ID NO: 235 - protein, 9 aa, synthetic construct
QQRSNWPIT

SEQ ID NO: 236 - protein, 116 aa, synthetic construct
QVQVQESGPGLVAPSQTLSITCTVSGFSLTTSGVSWVRQPPGKGLEWLGVIWGDGSTNYHPSLKSRLSIKKDHSKSQVFLKLNSLTAADTATYYCAKGGYSLAHWGQGTLVTVSSA

SEQ ID NO: 237 - protein, 108 aa, synthetic construct
DIQMTQSPSSLSVSVGERVTITCRASENIRSNLAWYQQKPGKSPKLLVNVATNLADGVPSRFSGSGSGTDYSLKINSLQPEDFGTYYCQHYWGTTWTFGQGTKLEIKR

SEQ ID NO: 238 - protein, 5 aa, synthetic construct
TSGVS

SEQ ID NO: 239 - protein, 16 aa, synthetic construct
VIWGDGSTNYHPSLKS

SEQ ID NO: 240 - protein, 7 aa, synthetic construct
GGYSLAH

SEQ ID NO: 241 - protein, 11 aa, synthetic construct
RASENIRSNLA

SEQ ID NO: 242 - protein, 7 aa, synthetic construct
VATNLAD

SEQ ID NO: 243 - protein, 9 aa, synthetic construct
QHYWGTTWT

SEQ ID NO: 244 - protein, 8 aa, synthetic construct
GFSLTTSG

SEQ ID NO: 245 - protein, 7 aa, synthetic construct
IWGDGST

SEQ ID NO: 246 - protein, 9 aa, synthetic construct
AKGGYSLAH

SEQ ID NO: 247 - protein, 6 aa, synthetic construct
ENIRSN

SEQ ID NO: 248 - protein, 9 aa, synthetic construct
QHYWGTTWT

SEQ ID NO: 249 - protein, 7 aa, synthetic construct
GFSLTTS

SEQ ID NO: 250 - protein, 5 aa, synthetic construct
WGDGS

SEQ ID NO: 251 - protein, 7 aa, synthetic construct
GGYSLAH

SEQ ID NO: 252 - protein, 11 aa, synthetic construct
RASENIRSNLA

SEQ ID NO: 253 - protein, 7 aa, synthetic construct
VATNLAD

SEQ ID NO: 254 - protein, 9 aa, synthetic construct
QHYWGTTWT

SEQ ID NO: 255 - protein, 119 aa, synthetic construct
EIQLQQSGPELVKPGASVKVSCKASGYSFTDYNMYWVKQSHGKSLEWIGYIDPYNGDTTYNQKFKGKATLTVDKSSSTAFIHLNSLTSEDSAVYYCARSPYGHYAMDYWGQGTSVTVSS

SEQ ID NO: 256 - protein, 108 aa, synthetic construct
DIVMTQSHKLLSTSVGDRVSITCKASQDVSTAVDWYQQKPGQSPKLLINWASTRHTGVPDRFTGSGSGTDYTLTISSMQAEDLALYYCRQHYSTPFTFGSGTKLEIKR

SEQ ID NO: 257 - protein, 5 aa, synthetic construct
DYNMY

SEQ ID NO: 258 - protein, 17 aa, synthetic construct
YIDPYNGDTTYNQKFKG

SEQ ID NO: 259 - protein, 10 aa, synthetic construct
SPYGHYAMDY

SEQ ID NO: 260 - protein, 11 aa, synthetic construct
KASQDVSTAVD

SEQ ID NO: 261 - protein, 7 aa, synthetic construct
WASTRHT

SEQ ID NO: 262 - protein, 9 aa, synthetic construct
RQHYSTPFT

SEQ ID NO: 263 - protein, 8 aa, synthetic construct
GYSFTDYN

SEQ ID NO: 264 - protein, 8 aa, synthetic construct
IDPYNGDT

SEQ ID NO: 265 - protein, 12 aa, synthetic construct
ARSPYGHYAMDY

SEQ ID NO: 266 - protein, 6 aa, synthetic construct
QDVSTA

SEQ ID NO: 267 - protein, 9 aa, synthetic construct
RQHYSTPFT

SEQ ID NO: 268 - protein, 7 aa, synthetic construct
GYSFTDY

SEQ ID NO: 269 - protein, 6 aa, synthetic construct
DPYNGD

SEQ ID NO: 270 - protein, 10 aa, synthetic construct
SPYGHYAMDY

SEQ ID NO: 271 - protein, 11 aa, synthetic construct
KASQDVSTAVD

SEQ ID NO: 272 - protein, 7 aa, synthetic construct
WASTRHT

SEQ ID NO: 273 - skipped

SEQ ID NO: 274 - protein, 116 aa, synthetic construct
QVQLQQWGAGLLKPSETLSLTCAVYGGSFSPYYWSWIRQPPGKGLEWIGEINHSGSTNYNPSLKSRVTISVDTSKNQFSLKLSSVTAADTAVYYCARRAGDFDYWGQGTLVTVSSA

SEQ ID NO: 275 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYIFGQGTKLEIKR

SEQ ID NO: 276 - protein, 5 aa, synthetic construct
PYYWS

SEQ ID NO: 277 - protein, 16 aa, synthetic construct
EINHSGSTNYNPSLKS

SEQ ID NO: 278 - protein, 7 aa, synthetic construct
RAGDFDY

SEQ ID NO: 279 - protein, 11 aa, synthetic construct
RASQSISSWLA

SEQ ID NO: 280 - protein, 7 aa, synthetic construct
KASSLES

SEQ ID NO: 281 - protein, 7 aa, synthetic construct
QQYNSYI

SEQ ID NO: 282 - protein, 8 aa, synthetic construct
GGSFSPYY

SEQ ID NO: 283 - protein, 7 aa, synthetic construct
INHSGST

SEQ ID NO: 284 - protein, 9 aa, synthetic construct
ARRAGDFDY

SEQ ID NO: 285 - protein, 6 aa, synthetic construct
QSISSW

SEQ ID NO: 286 - protein, 7 aa, synthetic construct
QQYNSYI

SEQ ID NO: 287 - protein, 7 aa, synthetic construct
GGSFSPY

SEQ ID NO: 288 - protein, 5 aa, synthetic construct
NHSGS

SEQ ID NO: 289 - protein, 7 aa, synthetic construct
RAGDFDY

SEQ ID NO: 290 - protein, 11 aa, synthetic construct
RASQSISSWLA

SEQ ID NO: 291 - protein, 7 aa, synthetic construct
KASSLES

SEQ ID NO: 292 - protein, 7 aa, synthetic construct
QQYNSYI

SEQ ID NO: 293 - protein, 117 aa, synthetic construct
EVQLVQSGAEVKKPGESLKISCKGSGYSFTGYNMNWVRQMPGKGLEWMGNIDPYYGGTTYNRKFKGQVTISADKSISTAYLQWSSLKASDTAMYYCARSVGPFDSWGQGTLVTVSSG

SEQ ID NO: 294 - protein, 107 aa, synthetic construct
EIVLTQSPATLSLSPGERATLSCRASENVYSYLAWYQQKPGQAPRLLIYFAKTLAEGIPARFSGSGSGTDFTLTISSLEPEDFAVYYCQHHSDNPWTFGQGTKVEIK

SEQ ID NO: 295 - protein, 5 aa, synthetic construct
GYNMN

SEQ ID NO: 296 - protein, 17 aa, synthetic construct
NIDPYYGGTTYNRKFKG

SEQ ID NO: 297 - protein, 7 aa, synthetic construct
SVGPFDS

SEQ ID NO: 298 - protein, 11 aa, synthetic construct
RASENVYSYLA

SEQ ID NO: 299 - protein, 7 aa, synthetic construct
FAKTLAE

SEQ ID NO: 300 - protein, 9 aa, synthetic construct
QHHSDNPWT

SEQ ID NO: 301 - protein, 8 aa, synthetic construct
GYSFTGYN

SEQ ID NO: 302 - protein, 8 aa, synthetic construct
IDPYYGGT

SEQ ID NO: 303 - protein, 9 aa, synthetic construct
ARSVGPFDS

SEQ ID NO: 304 - protein, 6 aa, synthetic construct
ENVYSY

SEQ ID NO: 305 - protein, 9 aa, synthetic construct
QHHSDNPWT

SEQ ID NO: 306 - protein, 7 aa, synthetic construct
GYSFTGY

SEQ ID NO: 307 - protein, 6 aa, synthetic construct
DPYYGG

SEQ ID NO: 308 - protein, 7 aa, synthetic construct
SVGPFDS

SEQ ID NO: 309 - protein, 11 aa, synthetic construct
RASENVYSYLA

SEQ ID NO: 310 - protein, 7 aa, synthetic construct
FAKTLAE

SEQ ID NO: 311 - protein, 9 aa, synthetic construct
QHHSDNPWT

SEQ ID NO: 312 - protein, 120 aa, synthetic construct; identical to SEQ ID NO: 46

SEQ ID NO: 313 - protein, 107 aa, synthetic construct; identical to SEQ ID NO: 47

SEQ ID NO: 314 - protein, 5 aa, synthetic construct
DYVIN

SEQ ID NO: 315 - protein, 17 aa, synthetic construct
EIYPGSGTNYYNEKFKA

SEQ ID NO: 316 - protein, 11 aa, synthetic construct
RGRYGLYAMDY

SEQ ID NO: 317 - protein, 11 aa, synthetic construct
RASQDISNYLN

SEQ ID NO: 318 - protein, 7 aa, synthetic construct
YTSRLHS

SEQ ID NO: 319 - protein, 9 aa, synthetic construct
QQGNTRPWT

SEQ ID NO: 320 - protein, 8 aa, synthetic construct
GYTFSDYV

SEQ ID NO: 321 - protein, 8 aa, synthetic construct
IYPGSGTN

SEQ ID NO: 322 - protein, 13 aa, synthetic construct
ARRGRYGLYAMDY

SEQ ID NO: 323 - protein, 6 aa, synthetic construct
QDISNY

SEQ ID NO: 324 - protein, 9 aa, synthetic construct
QQGNTRPWT

SEQ ID NO: 325 - protein, 7 aa, synthetic construct
GYTFSDY

SEQ ID NO: 326 - protein, 6 aa, synthetic construct
YPGSGT

SEQ ID NO: 327 - protein, 11 aa, synthetic construct
RGRYGLYAMDY

SEQ ID NO: 328 - protein, 11 aa, synthetic construct
RASQDISNYLN

SEQ ID NO: 329 - protein, 7 aa, synthetic construct
YTSRLHS

SEQ ID NO: 330 - protein, 9 aa, synthetic construct
QQGNTRPWT

SEQ ID NO: 331 - protein, 122 aa, synthetic construct
QVQLQESGPGLVKPSQTLSLTCTVSGYSISSDYAWNWIRQPPGKGLEWIGYITYSGSTSYNPSLESRVTISRDTSKNQFSLKLSSVTAADTAVYYCARGGYYGSSWGVFAYWGQGTLVTVSS

SEQ ID NO: 332 - protein, 107 aa, synthetic construct
DIQMTQSPSSLSASVGDRVTITCRVSENIYSYLAWYQQKPGKAPKLLVYNAKTLAEGVPSRFSGSGSGTDFTLTISSLQPEDFATYYCQHHYGTPWTFGGGTKVEIK

SEQ ID NO: 333 - protein, 6 aa, synthetic construct
SDYAWN

SEQ ID NO: 334 - protein, 16 aa, synthetic construct
YITYSGSTSYNPSLES

SEQ ID NO: 335 - protein, 13 aa, synthetic construct
GGYYGSSWGVFAY

SEQ ID NO: 336 - protein, 11 aa, synthetic construct
RVSENIYSYLA

SEQ ID NO: 337 - protein, 7 aa, synthetic construct
NAKTLAE

SEQ ID NO: 338 - protein, 9 aa, synthetic construct
QHHYGTPWT

SEQ ID NO: 339 - protein, 9 aa, synthetic construct
GYSISSDYA

SEQ ID NO: 340 - protein, 7 aa, synthetic construct
ITYSGST

SEQ ID NO: 341 - protein, 15 aa, synthetic construct
ARGGYYGSSWGVFAY

SEQ ID NO: 342 - protein, 6 aa, synthetic construct
ENIYSY

SEQ ID NO: 343 - protein, 9 aa, synthetic construct
QHHYGTPWT

SEQ ID NO: 344 - protein, 8 aa, synthetic construct
GYSISSDY

SEQ ID NO: 345 - protein, 5 aa, synthetic construct
TYSGS

SEQ ID NO: 346 - protein, 13 aa, synthetic construct
GGYYGSSWGVFAY

SEQ ID NO: 347 - protein, 11 aa, synthetic construct
RVSENIYSYLA

SEQ ID NO: 348 - protein, 7 aa, synthetic construct
NAKTLAE

SEQ ID NO: 349 - protein, 9 aa, synthetic construct
QHHYGTPWT

SEQ ID NO: 350 - protein, 116 aa, synthetic construct; identical to SEQ ID NO: 65

SEQ ID NO: 351 - protein, 107 aa, synthetic construct; identical to SEQ ID NO: 66

SEQ ID NO: 352 - protein, 5 aa, synthetic construct
EYTMH

SEQ ID NO: 353 - protein, 17 aa, synthetic construct
GISPNIGGTSYNQKFKG

SEQ ID NO: 354 - protein, 7 aa, synthetic construct
RGGSFDY

SEQ ID NO: 355 - protein, 11 aa, synthetic construct
RASQSISDYLH

SEQ ID NO: 356 - protein, 7 aa, synthetic construct
YASQSIS

SEQ ID NO: 357 - protein, 9 aa, synthetic construct
QNGHSFPLT

SEQ ID NO: 358 - protein, 8 aa, synthetic construct
GYTFSEYT

SEQ ID NO: 359 - protein, 8 aa, synthetic construct
ISPNIGGT

SEQ ID NO: 360 - protein, 9 aa, synthetic construct
ARRGGSFDY

SEQ ID NO: 361 - protein, 6 aa, synthetic construct
QSISDY

SEQ ID NO: 362 - protein, 9 aa, synthetic construct
QNGHSFPLT

SEQ ID NO: 363 - protein, 7 aa, synthetic construct
GYTFSEY

SEQ ID NO: 364 - protein, 6 aa, synthetic construct
SPNIGG

SEQ ID NO: 365 - protein, 7 aa, synthetic construct
RGGSFDY

SEQ ID NO: 366 - protein, 11 aa, synthetic construct
RASQSISDYLH

SEQ ID NO: 367 - protein, 7 aa, synthetic construct
YASQSIS

SEQ ID NO: 368 - protein, 9 aa, synthetic construct
QNGHSFPLT

SEQ ID NO: 369 - protein, 117 aa, synthetic construct
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSS

SEQ ID NO: 370 - protein, 107 aa, synthetic construct
DIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIK

SEQ ID NO: 371 - protein, 5 aa, synthetic construct
SFTMH

SEQ ID NO: 372 - protein, 17 aa, synthetic construct
YINPSSGYTEYNQKFKD

SEQ ID NO: 373 - protein, 8 aa, synthetic construct
GSSRGFDY

SEQ ID NO: 374 - protein, 11 aa, synthetic construct
RASENIYSNLA

SEQ ID NO: 375 - protein, 7 aa, synthetic construct
AATNLAD

SEQ ID NO: 376 - protein, 9 aa, synthetic construct
QHFWGTPRT

SEQ ID NO: 377 - protein, 8 aa, synthetic construct
GYTFTSFT

SEQ ID NO: 378 - protein, 8 aa, synthetic construct
INPSSGYT

SEQ ID NO: 379 - protein, 10 aa, synthetic construct
VRGSSRGFDY

SEQ ID NO: 380 - protein, 6 aa, synthetic construct
ENIYSN

SEQ ID NO: 381 - protein, 9 aa, synthetic construct
QHFWGTPRT

SEQ ID NO: 382 - protein, 7 aa, synthetic construct
GYTFTSF

SEQ ID NO: 383 - protein, 6 aa, synthetic construct
NPSSGY

SEQ ID NO: 384 - protein, 8 aa, synthetic construct
GSSRGFDY

SEQ ID NO: 385 - protein, 11 aa, synthetic construct
RASENIYSNLA

SEQ ID NO: 386 - protein, 7 aa, synthetic construct
AATNLAD

SEQ ID NO: 387 - protein, 9 aa, synthetic construct
QHFWGTPRT

SEQ ID NO: 388 - protein, 118 aa, synthetic construct
EVQLQQSGAEVKKPGSSVKVSCKTSGYTFTEYSMHWVRQAPGKSLEWFGGISPNSGGTSYNQKFKGRATLTVDKSSSTAYMELSSLRSEDTAVYYCARRDFHSSFDYWGQGTTVTVSS

SEQ ID NO: 389 - protein, 107 aa, synthetic construct
DIVMTQSPATLSLSPGERATLSCRASQSISDYLHWFQQKPHESPRLLIKYASQSISGIPARFSGSGSGSDFTLTISSLEPEDFAVYYCQNGHSFPLTFGPGTKVDIK

SEQ ID NO: 390 - protein, 5 aa, synthetic construct
EYSMH

SEQ ID NO: 391 - protein, 17 aa, synthetic construct
GISPNSGGTSYNQKFKG

SEQ ID NO: 392 - protein, 9 aa, synthetic construct
RDFHSSFDY

SEQ ID NO: 393 - protein, 11 aa, synthetic construct
RASQSISDYLH

SEQ ID NO: 394 - protein, 7 aa, synthetic construct
YASQSIS

SEQ ID NO: 395 - protein, 9 aa, synthetic construct
QNGHSFPLT

SEQ ID NO: 396 - protein, 8 aa, synthetic construct
GYTFTEYS

SEQ ID NO: 397 - protein, 8 aa, synthetic construct
ISPNSGGT

SEQ ID NO: 398 - protein, 11 aa, synthetic construct
ARRDFHSSFDY

SEQ ID NO: 399 - protein, 6 aa, synthetic construct
QSISDY

SEQ ID NO: 400 - protein, 9 aa, synthetic construct
QNGHSFPLT

SEQ ID NO: 401 - protein, 7 aa, synthetic construct
GYTFTEY

SEQ ID NO: 402 - protein, 6 aa, synthetic construct
SPNSGG

SEQ ID NO: 403 - protein, 9 aa, synthetic construct
RDFHSSFDY

SEQ ID NO: 404 - protein, 11 aa, synthetic construct
RASQSISDYLH

SEQ ID NO: 405 - protein, 7 aa, synthetic construct
YASQSIS

SEQ ID NO: 406 - protein, 9 aa, synthetic construct
QNGHSFPLT

SEQ ID NO: 407 - protein, 120 aa, synthetic construct
EVQLVESGGGLVQPGGSLRLSCAASGFSLSNYNMGWVRQAPGKGLEWVSVIDASGTTYYATWAKGRFTISRDNSKNTLYLQMNSLRAEDTATYYCARELLYFGSSYYDLWGQGTLVTVSS

SEQ ID NO: 408 - protein, 110 aa, synthetic construct
DVVMTQSPSTLSASVGDRVTITCQASQNIDSNLAWYQQKPGKAPKFLIYYASNLPFGVPSRFKGSGSGTEFTLTISSLQPDDFATYYCQSADVGSTYVAAFGGGTKVEIK

SEQ ID NO: 409 - protein, 5 aa, synthetic construct
NYNMG

SEQ ID NO: 410 - protein, 16 aa, synthetic construct
VIDASGTTYYATWAKG

SEQ ID NO: 411 - protein, 12 aa, synthetic construct
ELLYFGSSYYDL

SEQ ID NO: 412 - protein, 11 aa, synthetic construct
QASQNIDSNLA

SEQ ID NO: 413 - protein, 7 aa, synthetic construct
YASNLPF

SEQ ID NO: 414 - protein, 12 aa, synthetic construct
QSADVGSTYVAA

SEQ ID NO: 415 - protein, 8 aa, synthetic construct
GFSLSNYN

SEQ ID NO: 416 - protein, 7 aa, synthetic construct
IDASGTT

SEQ ID NO: 417 - protein, 14 aa, synthetic construct
ARELLYFGSSYYDL

SEQ ID NO: 418 - protein, 6 aa, synthetic construct
QNIDSN

SEQ ID NO: 419 - protein, 12 aa, synthetic construct
QSADVGSTYVAA

SEQ ID NO: 420 - protein, 7 aa, synthetic construct
GFSLSNY

SEQ ID NO: 421 - protein, 5 aa, synthetic construct
DASGT

SEQ ID NO: 422 - protein, 12 aa, synthetic construct
ELLYFGSSYYDL

SEQ ID NO: 423 - protein, 11 aa, synthetic construct
QASQNIDSNLA

SEQ ID NO: 424 - protein, 7 aa, synthetic construct
YASNLPF

SEQ ID NO: 425 - protein, 12 aa, synthetic construct
QSADVGSTYVAA

SEQ ID NO: 426 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 427 - protein, 330 aa, synthetic construct [sequence omitted]

SEQ ID NO: 428 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 429 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 430 - protein, 107 aa, synthetic construct [sequence omitted]

SEQ ID NO: 431 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 432 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 433 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 434 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 435 - protein, 106 aa, synthetic construct
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS